(19) United States
(12) Patent Application Publication
(10) Pub. No.: US 2026/0306188 A1
(43) Pub. Date: Oct. 8, 2026

(54) Methods for Advanced Intraoperative Imaging

(71) Applicant: The Regents of the University of California, Oakland, CA (US)

(72) Inventor: Mekhail Anwar, Berkeley, CA (US)

(21) Appl. No.: 19/535,951

(22) Filed: Feb. 10, 2026

Related U.S. Application Data
(60) Provisional application No. 63/758,205, filed on Feb. 13, 2025.
(60) Provisional application No. 63/868,110, filed on Aug. 21, 2025.

(51) Int. Cl. A61B 34/20; G01T 1/20

(52) U.S. Cl. CPC A61B 34/20; G01T 1/2018

(57) ABSTRACT

Devices, systems, and methods are provided for intraoperative imaging using chip-based imagers that are capable of detecting both fluorescence and gamma radiation. Also provided are molecular probes capable of emitting both fluorescence and gamma radiation for use in labeling cells. The chip-based imagers and probes can be used for detecting multiple cell types within diseased tissue or a tumor. Methods of using the disclosed chip-based imagers and probes for combined fluorescence-guided and radio-guided surgery are also provided. The disclosed methods can be used in surgical resection of a tumor to avoid overlooking small areas of tumor buried under tissue, metastases, and microscopic disease, and to assess surgical margins.

### CROSS-REFERENCE TO RELATED APPLICATION

This application claims benefit under 35 U.S.C. § 119 (e) of provisional applications 63/758,205, filed Feb. 13, 2025, and 63/868,110, filed Aug. 21, 2025, which applications are hereby incorporated by reference in their entirety.

### BACKGROUND OF THE INVENTION

Approximately 60% of cancer patients undergo surgery to remove a primary tumor, and precise removal of all cancer cells is vital for optimal outcomes (Azari et al., J. Biomed Opt 26 (2021); Nguyen, Intraoperative PSMA florescence: guided, robotic radical prostatectomy: results of a phase one study. in Molecular-Guided Surgery: Molecules, Devices, and Applications VIII (eds. Gibbs, S. L., Pogue, B. W. & Gioux, S.) 36 (SPIE, 2022). doi: 10.1117/12.2619285). Microscopic clusters of cancer cells are intraoperatively hard to visualize and are often left behind, significantly increasing the risk of cancer recurrence. In addition to optical intraoperative imaging, radioguided surgery (RGS) is one technique that has been shown to help prevent this. This method involves conjugating a gamma photon (γ)-emitting radioisotope to a cancer-targeting molecule and administering this radioactive tracer intravenously to the patient before surgery where it preferentially binds to cancer cells. However, RGS suffers from limited spatial resolution and is not optimal for margin imaging, but is better geared towards detecting clusters of cells below the tissue surface.

There remains a need for better methods of intraoperative imaging that not only enable imaging of the tissue surface but also extend imaging to millimeters to centimeters below the tissue surface.

### SUMMARY OF THE INVENTION

Devices, systems, and methods are provided for intraoperative imaging using chip-based imagers that are capable of detecting both fluorescence and gamma radiation. Also provided are molecular probes capable of emitting both fluorescence and gamma radiation for use in labeling cells. The chip-based imagers and probes can be used for detecting multiple cell types within diseased tissue or a tumor. Methods of using the disclosed chip-based imagers and probes for combined fluorescence-guided and radio-guided surgery are also provided. The disclosed methods can be used in surgical resection of a tumor to avoid overlooking small areas of tumor buried under tissue, metastases, and microscopic disease, and to assess surgical margins.

In one aspect, a dual modality imager is provided, the dual modality imager comprising: a first chip comprising an array of gamma photon-sensing pixels, wherein each gamma photon-sensing pixel provides in-pixel signal amplification for incoming gamma photons that hit the gamma photon-sensing pixel and in-pixel data processing to determine relative energies and angles of incidence of the incoming gamma photons, and wherein each gamma photon-sensing pixel only outputs data from the in-pixel data processing in response to an incoming gamma photon hitting the gamma photon-sensing pixel; and a second chip comprising an array of fluorescence-sensing pixels, wherein the second chip is positioned relative to the first chip such that the incoming gamma photons pass through the second chip to reach the array of gamma photon-sensing pixels.

In certain embodiments, the dual modality imager can be used to perform simultaneous imaging of gamma photons and fluorescence.

In certain embodiments, the dual modality imager can be used to perform intraoperative image-guided surgery.

In certain embodiments, the in-pixel data processing further determines tissue depths of gamma-emitting radionuclide sources from which the incoming gamma photons originate.

In certain embodiments, the imaging of gamma photons is single photon emission computed tomography (SPECT) imaging.

In certain embodiments, the fluorescence-sensing pixels detect fluorescent light in a near-infrared or visible region of the electromagnetic spectrum.

In certain embodiments, the dual modality imager further comprises a handle extending away and disposed angularly from the first chip.

In certain embodiments, each gamma photon-sensing pixel comprises a silicon diode, wherein a gamma photon incident on the surface of the silicon diode breaks a silicon bond to produce an electron-hole pair, wherein a pulse of charge (Qₚ) is generated that is accumulated by a diode capacitor resulting in generation of a voltage pulse.

In certain embodiments, the array of gamma photon-sensing pixels comprises a plurality of energy-resolving pixels, a plurality of low-flux pixels, and a plurality of energy-calibrating pixels, wherein each energy-resolving pixel comprises a reverse-biased diode that generates a voltage drop at a diode node in response to energy deposition by an incoming gamma photon hitting the reverse-biased diode, wherein the reverse-biased diode is connected to a unity-gain buffer, wherein output of the unity-gain buffer is connected to an operational transconductance amplifier (OTA) with a direct current (DC) compensation loop that secures an output DC level of the OTA, wherein output of the OTA is routed to a p-channel metal-oxide-semiconductor (PMOS) common-source (CS) stage with a configurable load to set a custom sensitivity of the energy-resolving pixel, wherein the load is configured with a 3-bit in-pixel six transistor (6T) static random-access memory (SRAM) that is used to control in-pixel digital-to-analog converters (DACs) that set a gate voltage of the load; and two inverters, wherein voltage pulses are digitized using the two inverters and sent to a digital processing unit via a set of switches, wherein widths of the digital pulses are measured to estimate a voltage signal decay time (DT); wherein each low-flux pixel comprises 6 reverse-biased diodes, wherein each reverse-biased diode is coupled to a unity-gain buffer, wherein buffer outputs from each unity-gain buffer are connected together and routed to an OTA comprising a unity-gain buffer in a feedback loop, said OTA serving as a pseudoresistor to create a summing amplifier with gain=−1, wherein summation of individual gamma photon hits on any of the 6 reverse-biased diodes is routed to an open-loop OTA comprising a DC compensation loop, N-type metal-oxide-semiconductor (NMOS) CS-stage with an SRAM-configurable load, and inverters for count digitization; and wherein the energy-calibrating pixels comprise a plurality of reverse-biased diodes of varying diode sizes having different diode parasitic capacitances and different voltage drops in response to energy deposition by an incoming gamma photon, wherein the plurality of reverse-biased diodes are connected to a unity-gain buffer, wherein output of the unity-gain buffer is connected to an OTA with a DC compensation loop that secures an output DC level of the OTA, wherein output of the OTA is routed to a PMOS CS stage with a configurable load to set a custom sensitivity of the energy-calibrating pixel, wherein the load is configured with a 3-bit in-pixel 6T SRAM that is used to control in-pixel DACs that set a gate voltage of the load; and two inverters, wherein voltage pulses are digitized using the two inverters and sent to a digital processing unit via a set of switches, wherein the energy-calibrating pixels are used for energy-binning to determine the maximum energy deposited (E_{dep}) by a gamma photon, wherein a least significant bit (LSB) is selected to optimize energy resolution and dynamic range, and wherein the LSB is used to estimate the DT.

In certain embodiments, the reverse-biased diodes of varying diode sizes of the energy-calibrating pixels comprise diode areas of 4.41 μm², 4.84 μm², 5.29 μm², 5.76 μm², and 6.25 μm².

In certain embodiments, the array of gamma photon-sensing pixels comprises a plurality of rows of energy-resolving pixels, a plurality of rows of low-flux pixels, and a plurality of rows of energy-calibrating pixels, wherein each row in the array of gamma photon-sensing pixels shares a count-line (CL) and a count column line (CCL), wherein the CL outputs a pulse whenever a gamma photon is detected by a gamma photon-sensing pixel, wherein numbers of pulses output by the CL are measured with a 10-bit counter, wherein non-overlapping column address sweep (CAS) signals are routed down every column of the array of gamma photon-sensing pixels such that when a gamma photon is detected, the CCL outputs a pulse to identify the column address, wherein the DT, column, and row data are priority encoded, queued into a first in first out (FIFO) memory element, serialized, and sent to a field programmable gate array (FPGA).

In certain embodiments, the dual modality imager further comprises an SRAM sensitivity calibration unit, wherein the SRAM sensitivity calibration unit is used to read or write to the 3-bit in-pixel SRAM to control gain of each CS-stage.

In certain embodiments, each gamma photon-sensing pixel is initially calibrated in absence of gamma radiation, wherein SRAM values are increased to increase the CS gain until the CL is triggered by noise, wherein a maximum CS gain threshold is selected at below the level at which the CL is triggered by noise.

In certain embodiments, each gamma photon-sensing pixel comprises: a silicon diode; a unity gain voltage amplifier connected to the silicon diode, wherein each voltage pulse generated by an incoming gamma photon is individually buffered by the unity gain voltage amplifier; a differential closed-loop amplifier, wherein buffered voltage outputs from the unity gain voltage amplifier are connected to inputs of the differential closed-loop amplifier, wherein voltage gain is either pre-set or configurable using in-pixel memory and a digital-to-analog converter (DAC), wherein the voltage pulse generated across the silicon diode is buffered and amplified by a fixed process-invariant gain; an inverter chain comprising a plurality of inverters connected to amplified voltage output from the differential closed-loop amplifier, wherein the inverter chain generates digitized output corresponding to each voltage pulse generated by a gamma photon; a digital counter coupled to the digitized output of the inverter chain, wherein the digital counter counts gamma photon detection events, wherein each gamma photon detection event corresponds to the voltage pulse produced by each gamma photon incident on the surface of the silicon diode; an on-chip data buffer configured to store a plurality of gamma photon count records for a plurality of gamma photon detection events; a digital clock configured to produce a clock signal on the chip, wherein the digital counter uses the clock signal to count numbers of gamma photon detection events per a period of time; an energy storage device located on the first chip or externally; and a custom digital logic circuit configured to control voltages and supply power from the energy storage device to the array of gamma photon-sensing pixels, the digital counter, and the digital clock.

In certain embodiments, each gamma photon-sensing pixel comprises a pair of silicon diodes comprising a first silicon diode and a second silicon diode, wherein a gamma photon incident on a surface of either the first silicon diode or the second silicon diode breaks a silicon bond to produce an electron-hole pair, wherein a pulse of charge (Qₚ) is generated that is accumulated by a diode capacitor resulting in generation of a voltage pulse. In some embodiments, the first chip further comprises: a unity gain voltage amplifier connected to each silicon diode, wherein each voltage pulse generated by an incoming gamma photon is individually buffered by the unity gain voltage amplifier; a differential amplifier, wherein buffered voltage outputs from the unity gain voltage amplifier are connected to inputs of the differential amplifier; a voltage integrator, wherein the voltage integrator accepts a selected DC voltage and sets voltage output of the differential amplifier, wherein the voltage integrator is bootstrapped from the differential amplifier output to the differential amplifier input in a negative feedback configuration; a pair of level shifters connected to output of the differential amplifier, wherein the pair of level shifters comprises two level shifters comprising a first level shifter that shifts a direct current (DC) level of the output of the differential amplifier up to amplify only the voltage pulse from the first diode, and a second level shifter that shifts the DC level of the differential amplifier output down to only amplify the voltage pulse from the second diode, wherein an amount the DC level is shifted is set using on-chip configurable memory and an in-pixel digital to analog converter (DAC) to convert stored bits into an analog shift in voltage; an inverter chain comprising a plurality of inverters connected to amplified and shifted voltage output from the pair of level shifters, wherein the inverter chain generates digitized output corresponding to each voltage pulse generated by an incoming gamma photon; a digital counter coupled to the digitized output of the inverter chain, wherein the digital counter counts gamma photon detection events, wherein each gamma photon detection event corresponds to the voltage pulse produced by each gamma photon incident on the surface of either the first silicon diode or the second silicon diode; an on-chip data buffer configured to store a plurality of gamma photon count records for a plurality of gamma photon detection events; a digital clock configured to produce a clock signal on the chip, wherein the digital counter uses the clock signal to count numbers of gamma photon detection events per a period of time; an energy storage device located on the first chip or externally; and a custom digital logic circuit configured to control voltages and supply power from the energy storage device to the array of gamma photon-sensing pixels, the digital counter, and the digital clock.

In certain embodiments, the silicon diodes are reversed biased or have zero voltage bias.

In certain embodiments, the first chip is covered with a Compton scattering material. In some embodiments, the Compton scattering material comprises lead, tungsten, or bismuth. In some embodiments, the Compton scattering material is in a layer on top of the first chip or on the back of the first chip.

In certain embodiments, the first chip further comprises on-chip circuitry tuned to be responsive to a range of linear energy transfer (LET) from incoming gamma photons such that combination and distribution of signals from gamma photon-sensing pixels with known LET responsivity enables determination of incident energies of the incoming gamma photons. In some embodiments, the array of gamma photon-sensing pixels comprises a first subset of gamma photon-sensing pixels and a second subset of gamma photon-sensing pixels with known LET responsivity, wherein the first subset of gamma photon-sensing pixels has a lower gain than the second subset of gamma photon-sensing pixels, wherein the first subset of gamma photon-sensing pixels detects lower energy photons having higher LET but not higher energy gamma photons having lower LET, wherein the second set of gamma photon-sensing pixels detects both the lower energy gamma photons having higher LET and the higher energy gamma photons having lower LET.

In certain embodiments, the gamma-emitting radionuclide is ⁴⁶Sc, ⁶⁴Cu, ⁶⁷Ga, ^{99m}Tc, ¹¹¹In, ¹²³I, ¹³¹I, ¹³³Ba, ¹⁵⁵Tb, ¹⁷⁷Lu, ¹³³Xe, or ²⁰¹TI.

In certain embodiments, each fluorescence-sensing pixel comprises a photodiode, wherein florescent light incident on a surface of the photodiode is converted into an electrical current proportional to intensity of the incident florescent light.

In certain embodiments, the photodiode is a silicon photodiode, a germanium photodiode, or an indium gallium arsenide photodiode.

In certain embodiments, the array of fluorescence-sensing pixels comprises a plurality of photodiodes arrayed on the surface of the second chip, wherein each photodiode is coated with at least one layer of filter material that functions as an optical filter.

In certain embodiments, the second chip further comprises: a plurality of light emitting sources to provide excitation light, wherein the plurality of light emitting sources are located on the second chip or externally; an energy storage device to supply power for operation of the second chip, wherein the energy storage device is located on the second chip or externally; a data storage unit in communication with the array of fluorescence-sensing pixels, wherein the data storage unit stores imaging data from the array of fluorescence-sensing pixels; and an application-specific integrated circuit (ASIC) configured to control voltages and supply power from the energy storage device to the array of fluorescence-sensing pixels, the plurality of light emitting sources on the second chip, and the data storage unit.

In certain embodiments, the light emitting sources are micro-laser diodes or light-emitting diodes.

In certain embodiments, the plurality of light-emitting diodes further comprises an emission filter on each light-emitting diode.

In certain embodiments, the on-chip energy storage device or the off-chip energy storage device comprises a battery, a capacitor, a radionuclide, a photovoltaic system, or a radionuclide in combination with a scintillator and photovoltaic energy harvester.

In certain embodiments, the plurality of light emitting sources located externally are implantable in tissue.

In certain embodiments, the plurality of light emitting sources located externally are micro-star light sources.

In certain embodiments, an external power source supplies power to the plurality of light emitting sources located on the second chip or externally.

In certain embodiments, at least one layer of filter material comprises or consists of amorphous silicon, crystalline silicon, gallium phosphide, cadmium selenide, gallium arsenide, or indium phosphide.

In certain embodiments, the thickness of the layer of filter material is varied on the plurality of photodiodes to allow selection of light at different fluorescence emission wavelengths for multiple fluorophores having different fluorescence emission spectra.

In certain embodiments, the fluorophore of interest has a fluorescence emission in the near-infrared or visible region of the electromagnetic spectrum, and the band gap and the thickness of the layer of filter material is chosen to allow selection of near-infrared light or visible light at the fluorescence emission wavelength of the fluorophore.

In certain embodiments, the optical filter is an absorption filter or an interference filter.

In certain embodiments, the absorption filter has a band gap and thickness suitable to allow light at a fluorescence emission wavelength of a fluorophore of interest to pass through to the photodiode.

In certain embodiments, the interference filter is a single bandpass, dual bandpass, triple bandpass, or quadruple bandpass interference filter.

In certain embodiments, the interference filter further comprises a layer of absorption filter material on top of the interference filter or underneath the interference filter.

In certain embodiments, the interference filter further comprises one or more layers of material comprising a plurality of angle selective gratings, collimators, or fiber optic plates that blocks light that is not incident within 30° of an axis perpendicular to the plane of the second chip.

In certain embodiments, the interference filter further comprises one or more layers of material comprising a plurality of fiber optic plates that blocks light that is not incident within 6° of an axis perpendicular to the plane of the second chip.

In certain embodiments, the interference filter further comprises one or more layers of material comprising a plurality of angle selective gratings that blocks light that is not incident within 15° of an axis perpendicular to the plane of the second chip.

In certain embodiments, one or more layers of material comprising a plurality of angle selective gratings, collimators, or fiber optics are on top of the layer of filter material, underneath the layer of filter material, or both on top and underneath the layer of filter material. In some embodiments, the layer on top of the layer of filter material blocks light that is not incident within 10°-15° of an axis perpendicular to the plane of the chip. In some embodiments, the layer underneath the layer of filter material blocks light that is not incident within 5°-30° of an axis perpendicular to the plane of the chip.

In another aspect, a computer-implemented method for integrating fluorescence and gamma-emitting radionuclide imaging data is provided, the computer performing steps comprising: receiving imaging data from the array of gamma photon-sensing pixels and the array of fluorescence-sensing pixels of the dual modality imager after administering a detectably effective amount of an imaging agent to a subject, wherein the imaging agent comprises a fluorophore and a gamma-emitting radionuclide conjugated to a binding agent that selectively binds to a cellular marker; and displaying an image showing the fluorescence detected by the fluorescence-sensing pixels and the gamma photons detected by the gamma photon-sensing pixels.

In certain embodiments, the method further comprises determining the size and depth of a lesion in tissue of the subject based on the relative energies of the incoming gamma photons measured by the gamma photon-sensing pixels. In some embodiments, the computer-implemented method further comprises displaying on the image the size and the depth of the lesion in the tissue.

In certain embodiments, the computer-implemented method further comprises determining relative energies and angles of incidence of the incoming gamma photons that hit the array of gamma photon-sensing pixels.

In certain embodiments, the computer-implemented method further comprises identifying the gamma-emitting radionuclide source of each incoming gamma photon that hits the array of gamma photon-sensing pixels.

In certain embodiments, the computer-implemented method further comprises quantifying energy deposition on a diode (E_{dep}) by an incoming gamma photon and voltage signal decay time (DT) after the incoming gamma photon hits the diode.

In another aspect, a non-transitory computer-readable medium is provided comprising program instructions that, when executed by a processor in a computer, causes the processor to perform a computer-implemented method for integrating fluorescence and gamma-emitting radionuclide imaging data, described herein.

In another aspect, a kit is provided comprising the non-transitory computer-readable medium, described herein, and instructions for using the dual modality imager for fluorescence and gamma-emitting radionuclide imaging. In certain embodiments, the kit further comprises a dual modality imager, described herein. In certain embodiments, the kit further comprises one or more imaging agents.

In another aspect, a system is provided, the system comprising: a dual modality imager, described herein; a processor, wherein the processor is programmed to integrate fluorescence and gamma-emitting radionuclide imaging data from the dual modality imager according to a computer implemented method described herein; an external data receiving device connected to the processor, wherein the external data receiving device receives the fluorescence and gamma-emitting radionuclide imaging data from the dual modality imager; and a display component that can display an image showing the fluorescence detected by the fluorescence-sensing pixels and the gamma photons detected by the gamma photon-sensing pixels of the dual modality imager.

In certain embodiments, the system further comprises an imaging agent comprising a fluorophore and a gamma-emitting radionuclide conjugated to a binding agent that selectively binds to a cellular marker.

In certain embodiments, the cellular marker is a tumor marker.

In certain embodiments, the cellular marker is a disease-associated marker.

In certain embodiments, the binding agent is an antibody, an antibody mimetic, an aptamer, or a ligand.

In certain embodiments, the imaging agent further comprises a nanoparticle, wherein the binding agent, fluorophore, and a gamma-emitting radionuclide are attached to the nanoparticle.

In certain embodiments, the system further comprises a plurality of imaging agents, wherein each imaging agent comprises a different binding agent that selectively binds to a different cellular marker, and wherein each imaging agent comprises a different fluorophore.

In certain embodiments, the system further comprises a first imaging agent comprising a fluorophore conjugated to a first binding agent that selectively binds to a first cellular marker and a second imaging agent comprising a gamma-emitting radionuclide conjugated to a second binding agent that selectively binds to a second cellular marker.

In another aspect, a method of performing intraoperative image-guided surgery on a subject is provided, the method comprising: administering a detectably effective amount of an imaging agent to the subject, wherein the imaging agent comprises a fluorophore and a gamma-emitting radionuclide conjugated to a binding agent that selectively binds to a cellular marker on a lesion; illuminating tissue with light at a fluorescence excitation wavelength of the fluorophore; performing image-guided surgical resection of the lesion using a dual modality imager, described herein, wherein the dual modality imager produces fluorescence and radionuclide images of the lesion; moving the dual modality imager around the tissue surrounding the resection site to produce fluorescence and radionuclide images of the tissue surrounding the resection site after said performing the image-guided surgical resection of the lesion; using the fluorescence and radionuclide images of the tissue surrounding the resection site to determine if the lesion was completely removed by the surgical resection and to detect additional lesions, if present, wherein the dual modality imager provides information regarding tissue depths and sizes of the additional lesions, if present; and surgically resecting any remaining portion of the lesion or additional lesions that are detected by the dual modality imager.

In certain embodiments, the method further comprises illuminating the surgical specimen removed from the subject with light at a fluorescence excitation wavelength of the fluorophore; moving the dual modality imager over surgical margins surrounding the lesion in the surgical specimen, wherein fluorescence and radionuclide images produced by the dual modality imager are used to detect cells of the lesion, if present, in the surgical margins to determine if the surgical specimen comprises a positive margin; and further checking the tissue surrounding the resection site in the subject for the cells of the lesion if the surgical specimen is determined to comprise a positive margin.

In certain embodiments, the lesion is a tumor, diseased tissue, a congenital malformation, or damaged or abnormal tissue.

In certain embodiments, the binding agent is an antibody, an antibody mimetic, an aptamer, or a ligand.

In certain embodiments, the radionuclide images are single photon emission computed tomography (SPECT) images.

In certain embodiments, the fluorophore emits fluorescent light in a near-infrared or visible region of the electromagnetic spectrum.

In certain embodiments, the method further comprises administering a plurality of imaging agents to the subject, wherein each imaging agent comprises a different binding agent that selectively binds to a different cellular marker, and wherein each imaging agent comprises a different fluorophore.

In certain embodiments, the method further comprises obtaining a pre-operative image of the subject after the subject is administered an imaging agent comprising a gamma-emitting radionuclide conjugated to a binding agent that selectively binds to the cellular marker on the lesion, wherein the pre-operative image shows locations of the gamma-emitting radionuclide in tissue of the subject.

In certain embodiments, the method further comprises attaching a gamma photon counter to the skin of the subject, wherein the gamma photon counter is used in combination with the dual modality imager to determine the location of the radionuclide source of the incoming gamma photons that hit a gamma photon-sensing pixel.

In another aspect, a method of performing intraoperative lymphatic imaging on a subject is provided, the method comprising: administering a detectably effective amount of an imaging agent to the subject, wherein the imaging agent comprises a fluorophore and a gamma-emitting radionuclide conjugated to a binding agent that selectively binds to a lymphatic marker; illuminating lymphatic tissue with light at a fluorescence excitation wavelength of the fluorophore; and performing image-guided lymphatic surgery using a dual modality imager, described herein, wherein the dual modality imager produces fluorescence and radionuclide images of the lymphatic system.

In certain embodiments, the dual modality imager produces an intraoperative map of lymphatic vessels for real-time surgical guidance.

In certain embodiments, the lymphatic surgery is a lymphovenous bypass, a nodal transplant, a lymphovenous anastomosis, or a surgical resection of a lymphatic tumor.

In certain embodiments, the lymphatic marker is a lymphatic vessel marker. In some embodiments, the lymphatic vessel marker is lymphatic vessel endothelial hyaluronan receptor 1 (LYVE-1), podoplanin (PDPN), Prospero homeobox 1 (PROX1), Fms-like tyrosine kinase receptor 4 (FLT-4), CD31, CD34, or D2-40, or any combination thereof.

In certain embodiments, the method further comprises administering a plurality of imaging agents to the subject, wherein each imaging agent comprises a different binding agent that selectively binds to a different cellular marker, and wherein each imaging agent comprises a different fluorophore

In certain embodiments, the method further comprises obtaining a pre-operative image of the subject after the subject is administered an imaging agent comprising a gamma-emitting radionuclide conjugated to a binding agent that selectively binds to the lymphatic marker, wherein the pre-operative image shows locations of the gamma-emitting radionuclide in the lymphatic system of the subject.

In certain embodiments, the method further comprises attaching a gamma photon counter to the skin of the subject, wherein the gamma photon counter is used in combination with the dual modality imager to determine the location of the radionuclide source of the incoming gamma photons that hit a gamma photon-sensing pixel.

In certain embodiments, the method further comprises moving the dual modality imager over the surgical site to produce fluorescence and radionuclide images of the surgical site after said performing the lymphatic surgery; assessing results of the lymphatic surgery using the fluorescence and radionuclide images of the surgical site; and performing further surgically repair of the lymphatic tissue, if needed, based on said assessing the results of the lymphatic surgery.

In certain embodiments, the method further comprises: administering a second imaging agent to the subject, wherein the second imaging agent comprises a second fluorophore and a second gamma-emitting radionuclide conjugated to a second binding agent that selectively binds to a vein marker, wherein the second fluorophore emits fluorescent light at a different wavelength than the fluorophore conjugated to the binding agent that selectively binds to the lymphatic marker; illuminating lymphatic tissue with light at a fluorescence excitation wavelength of the second fluorophore; and performing the image-guided lymphatic surgery using the dual modality imager, wherein the dual modality imager produces fluorescence and radionuclide images of veins and lymphatic system vessels.

In certain embodiments, the dual modality imager produces an intraoperative map of veins and lymphatic vessels for real-time surgical guidance.

In certain embodiments, the vein marker is nuclear receptor subfamily 2 group F member 2 (NR2F2), EPH receptor B4 (EPHB4), SMARCA4 SWI/SNF related BAF chromatin remodeling complex subunit ATPase 4 (SMARCA4), or any combination thereof.

In another aspect, a method of performing intraoperative imaging of an artery or a vein in a subject is provided, the method comprising: administering a detectably effective amount of an imaging agent to the subject, wherein the imaging agent comprises a fluorophore and a gamma-emitting radionuclide conjugated to a binding agent that selectively binds to an artery marker or a vein marker; illuminating the artery or vein with light at a fluorescence excitation wavelength of the fluorophore; and performing image-guided surgery on the artery or vein using the dual modality imager described herein, wherein the dual modality imager produces fluorescence and radionuclide images of the artery or vein.

In certain embodiments, the method further comprises moving the dual modality imager over a surgical site to produce fluorescence and radionuclide images of the surgical site after said performing the surgery on the artery or vein; assessing results of the surgery on the artery or vein using the fluorescence and radionuclide images of the surgical site; and performing further surgically repair of the artery or vein, if needed, based on assessing the results of surgery on the artery or vein.

In certain embodiments, the vein marker is receptor subfamily 2 group F member 2 (NR2F2), EPH receptor B4 (EPHB4), SMARCA4 SWI/SNF related BAF chromatin remodeling complex subunit ATPase 4 (SMARCA4), or any combination thereof.

In certain embodiments, the artery marker is kinase insert domain receptor (KDR), notch receptor 1 (NOTCH1), notch receptor 3 (NOTCH3), notch receptor 4 (NOTCH4), delta like canonical Notch ligand 4 (DELTA4), jagged canonical Notch ligand 1 (JAG1), jagged canonical Notch ligand 2 (JAG2), ephrin-B2 (EFNB2), or any combination thereof.

### BRIEF DESCRIPTION OF THE DRAWINGS

FIG. 1. ASIC-assisted RGS: (a) γ-emitting radiotracer administration and tumor resection, (b) γ spectrometer ASIC finds cancer cells, (c) single γ energy resolution.

FIG. 2. Current SoC and Envisioned 3D imaging.

FIG. 3. Prototypes: (a) SENTRI custom sensor showing central pixel array, on chip memory, digital clock and control circuits. (b) SENTRI v2 fabricated in XFAB 180 nm technology, with increased pixel array size, increased fill factor, improved pixel design, and energy spectrometry. Both chips designed in the Anwar Lab. (c) SENTRI v1 and v2.

FIG. 4. ¹⁷⁷Lu Titration with SENTRI v2 Chip (a) SENTRI v2 on PC Board and vial of ¹⁷⁷Lu (b) counts per minute (CPM) recorded per row and scaled by 80 rows. (c) SENTRI v2 CPM signal vs distance from showing 10 CPM/chip at 6 cm (in air).

FIG. 5. Experimental results: (a) linearity of ASIC with Cu-64 activity, (b) ASIC tracks radioactive decay of Cu-64, (c) counts response per row to Cu-64, (d) TOPAS versus measured E_{dep} distributions, (e) resolving incident γ energies using TOPAS.

FIG. 6. VISION Chip-scale NIR fluorescence imager.

FIG. 7. γ energy spectrum through 1 cm of H₂O provides depth information.

FIG. 8. Anatomy from the CT, Distributed Point Source Model, and Comparison to SPECT:

Reconstruction: (a) at 0 hours immediately after injection, (b) at 6 hrs post-injection (hpi), and (c) at 48 hpi using γ sparse reconstruction of molecularly targeted radionuclides.

FIG. 9. RGS pixel architectures: (a) energy-resolving pixels, (b) low-flux pixels, (c) energy-calibrating pixels, (d) analog building blocks common to all pixel architectures.

FIG. 10. γ spectrometer ASIC system architecture including configurable pixel array, SRAM sensitivity calibration unit, and decay time processing unit.

FIG. 11. Experimental setup for ASIC validation: (a) diagram of setup, (b) experimental photos.

FIG. 12. Die photo with annotated system blocks.

### DETAILED DESCRIPTION OF THE INVENTION

Devices, systems, and methods are provided for intraoperative imaging using chip-based imagers that are capable of detecting both fluorescence and gamma radiation. Also provided are molecular probes capable of emitting both fluorescence and gamma radiation for use in labeling cells. The chip-based imagers and probes can be used for detecting multiple cell types within diseased tissue or a tumor. Methods of using the disclosed chip-based imagers and probes for combined fluorescence-guided and radio-guided surgery are also provided.

Before the present devices, systems, and methods are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the fluorophore” includes reference to one or more fluorophores and equivalents thereof, e.g., fluorescent dyes, fluorescent labels, and the like, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about”, particularly in reference to a given quantity, is meant to encompass deviations of plus or minus five percent.

“Isolated” refers to an entity of interest that is in an environment different from that in which it may naturally occur. “Isolated” is meant to include entities that are within samples that are substantially enriched for the entity of interest and/or in which the entity of interest is partially or substantially purified.

The term “conjugated” refers to the joining by covalent or noncovalent means of two compounds or agents (e.g., an imaging agent comprising a binding agent conjugated to a fluorophore and/or a gamma-emitting radionuclide).

The terms “peptide”, “oligopeptide”, “polypeptide”, and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymers. Both full-length proteins and fragments thereof are encompassed by the definition. The terms also include post-expression modifications of the polypeptide, for example, phosphorylation, glycosylation, acetylation, hydroxylation, oxidation, and the like as well as chemically or biochemically modified or derivatized amino acids and polypeptides having modified peptide backbones. The terms also include fusion proteins, including, but not limited to, fusion proteins with a heterologous amino acid sequence, fusions with heterologous and homologous leader sequences, with or without N-terminal methionine residues; immunologically tagged proteins; and the like. The terms include polypeptides including one or more of a fatty acid moiety, a lipid moiety, a sugar moiety, and a carbohydrate moiety.

“Substantially purified” generally refers to isolation of a substance (compound, protein, nucleic acid, nanoparticles) such that the substance comprises the majority percent of the sample in which it resides. Typically in a sample, a substantially purified component comprises 50%, preferably 80%-85%, more preferably 90-95% of the sample. Techniques for purifying substances of interest are well-known in the art and include, for example, ion-exchange chromatography, affinity chromatography and sedimentation according to density.

The terms “fluorophore”, “fluorescent label”, and “fluorescer” are used interchangeably and refer to a molecule that can emit fluorescent light in response to illumination with light at an excitation wavelength. Exemplary fluorophores with emissions in the visible region of the light spectrum include, without limitation, SYBR green, SYBR gold, a CAL Fluor dye such as CAL Fluor Gold 540, CAL Fluor Orange 560, CAL Fluor Red 590, CAL Fluor Red 610, and CAL Fluor Red 635, a Quasar dye such as Quasar 570, Quasar 670, and Quasar 705, an Alexa Fluor such as Alexa Fluor 488, Alexa Fluor 546, Alexa Fluor 555, Alexa Fluor 594, Alexa Fluor 647, and Alexa Fluor 784, a cyanine dye such as Cy3, Cy3.5, Cy5, Cy5.5, and Cy7, fluorescein, 2′, 4′, 5′, 7′-tetrachloro-4-7-dichlorofluorescein (TET), carboxyfluorescein (FAM), fluorescein isothiocyanate (FITC), 6-carboxy-4′,5′-dichloro-2′,7′-dimethoxyfluorescein (JOE), hexachlorofluorescein (HEX), rhodamine, carboxy-X-rhodamine (ROX), tetramethyl rhodamine (TAMRA), and Texas Red. Exemplary near-infrared (NIR) fluorophores include, without limitation, IRDye dyes (e.g., IRDye 800CW, IRDye 680RD, IRDye 700, IRDye 750, and IRDye 800RS), CF dyes (e.g., CF680, CF680R, CF750, CF770, and CF790), Tracy dyes (e.g., Tracy 645 and Tracy 652), Alexa dyes (e.g., Alexa Fluor® 660 dye, Alexa Fluor® 700 dye, Alexa Fluor® 750 dye, and Alexa Fluor® 790), cyanine dyes (e.g., Cy7 and Cy7.5), thienothiadiazole dyes, phthalocyanine dyes, squaraine dyes, rhodamine dyes and analogues (e.g., Si-pyronine, Si-rhodamine, Te-rhodamine, and Changsha), borondipyrromethane (BODIPY) dyes, seminaphthofluorone xanthene dyes, benzo[c] heterocycle dyes (e.g., isobenzofuran dyes), and quantum dots. For a review of NIR fluorophores and their use in fluorescence imaging, see, e.g., Escobedo et al. (2010) Curr. Opin. Chem. Biol. 14 (1): 64-70, Hilderbrand et al. (2010) Curr. Opin. Chem. Biol. 14 (1): 71-79, Yuan et al. (2013) Chem. Soc. Rev. 42 (2): 622-661, Vats et al. (2017) Int. J. Mol. Sci. 18 (5), Zhang et al. (2017) Nat. Rev. Clin. Oncol. 14 (6): 347-364, Gao et al. (2010) Curr. Top. Med. Chem. 10 (12): 1147-1157, Zhao et al. (2018) Wiley Interdiscip Rev Nanomed Nanobiotechnol. 10 (3): e1483, and Haque et al. (2017) Bioorg. Med. Chem. 25 (7): 2017-2034; herein incorporated by reference in their entireties.

The term “fluorescence characteristics” means an ability to emit fluorescence in response to illumination with excitation light. Examples of parameters of fluorescence characteristics include fluorescence intensity, excitation wavelength, fluorescence wavelength, and pH sensitivity.

The terms “radionuclide”, “radioisotope”, “radioactive nuclide”, and “radioactive isotope” are used interchangeably and refer to an atom with an unstable nucleus that emits ionizing radiation or particles. Radionuclides may emit gamma radiation, alpha particles, beta particles, or positrons. In particular, the terms include positron-emitting radionuclides such as, but not limited to, ⁶⁴Cu, ⁸⁹Zr, ⁶⁸Ga, ¹⁷⁷Lu, ⁸²Rb, ⁸⁶Y, ¹¹C, ¹³N, ¹⁵O, and ¹⁸F; and gamma-emitting radionuclides such as, but not limited to, ⁴⁶Sc, ⁶⁴Cu, ⁶⁷Ga, ^{99m}Tc, ¹¹¹In, ¹²³I, ¹³¹I, ¹³³Ba, ¹⁵⁵Tb, ¹⁷⁷Lu, ¹³³Xe, and ²⁰¹TI. The terms may also include alpha-emitting radionuclides such as, but not limited to, ¹⁴⁹Tb, ²²³Ra, or ²²⁵Ac and beta-emitting radionuclides such as, but not limited to, ³²P, ⁹⁰Y, ¹³¹I, ⁸⁹Sr, ⁹⁹Tc, ¹⁵²Tb, ¹⁵³Sm, ¹⁶¹Tb, ¹⁶⁶Ho, and ¹⁷⁷Lu, which are suitable for radionuclide therapy. In addition, the terms include radionuclides used in theranostic agents, which can be used for both therapy and imaging. Such theranostic agents may include a radionuclide that can be used for both imaging and radiotherapy (e.g., iodine-131 and lutetium-177 are gamma and beta emitters, which can be used for both imaging and therapy) or a theranostic agent comprising both a diagnostic radionuclide and a therapeutic radionuclide. Alternatively, a theranostic agent may comprise a radionuclide linked to a non-radioactive therapeutic agent (e.g., radiopharmaceuticals, radioimmunotherapeutic agents, and radiopeptides).

The terms “tumor,” “cancer” and “neoplasia” are used interchangeably and refer to a cell or population of cells whose growth, proliferation or survival is greater than growth, proliferation or survival of a normal counterpart cell, e.g., a cell proliferative, hyperproliferative or differentiative disorder. Typically, the growth is uncontrolled. The term “malignancy” refers to invasion of nearby tissue. The term “metastasis” or a secondary, recurring or recurrent tumor, cancer or neoplasia refers to spread or dissemination of a tumor, cancer or neoplasia to other sites, locations or regions within the subject, in which the sites, locations or regions are distinct from the primary tumor or cancer. Neoplasia, tumors, and cancers include benign, malignant, metastatic and non-metastatic types, and include any stage (I, II, III, IV or V) or grade (G1, G2, G3, etc.) of neoplasia, tumor, or cancer, or a neoplasia, tumor, cancer or metastasis that is progressing, worsening, stabilized or in remission. In particular, the terms “tumor,” “cancer” and “neoplasia” include carcinomas, such as squamous cell carcinoma, adenocarcinoma, adenosquamous carcinoma, anaplastic carcinoma, large cell carcinoma, and small cell carcinoma, and include cancers such as, but are not limited to, pancreatic cancer, lung cancer (non-small cell lung cancer, small cell lung cancer), gastric cancer, ovarian cancer, endometrial cancer, colorectal cancer, oral cancer, skin cancer, cholangiocarcinoma, head and neck cancer, breast cancer, ovarian cancer, melanoma, peripheral neuroma, glioblastoma, adrenocortical carcinoma, AIDS-related lymphoma, anal cancer, bladder cancer, meningioma, glioma, astrocytoma, cervical cancer, chronic myeloproliferative disorders, colon cancer, endometrial cancer, ependymoma, esophageal cancer, Ewing's sarcoma, extracranial germ cell tumors, extrahepatic bile duct cancer, gallbladder cancer, gastrointestinal carcinoid tumors, gestational trophoblastic tumors, hairy cell leukemia, Hodgkin lymphoma, non-Hodgkin lymphoma, hypopharyngeal cancer, islet cell carcinoma, Kaposi sarcoma, laryngeal cancer, leukemia, lip cancer, oral cavity cancer, liver cancer, malignant mesothelioma, medulloblastoma, Merkel cell carcinoma, metastatic squamous neck cell carcinoma, multiple myeloma and other plasma cell neoplasms, mycosis fungoides and the Sezary syndrome, myelodysplastic syndromes, nasopharyngeal cancer, neuroblastoma, oropharyngeal cancer, bone cancers, including osteosarcoma and malignant fibrous histiocytoma of bone, paranasal sinus cancer, parathyroid cancer, penile cancer, pheochromocytoma, pituitary tumors, prostate cancer, rectal cancer, renal cell cancer, retinoblastoma, rhabdomyosarcoma, salivary gland cancer, small intestine cancer, soft tissue sarcoma, supratentorial primitive neuroectodermal tumors, pineoblastoma, testicular cancer, thymoma, thymic carcinoma, thyroid cancer, transitional cell cancer of the renal pelvis and ureter, urethral cancer, uterine sarcoma, vaginal cancer, vulvar cancer, and Wilm's tumor and other childhood kidney tumors.

A “ligand” or “binding agent” is any molecule that can be used to target an imaging agent (e.g., comprising a fluorophore and/or gamma-emitting radionuclide) to a cell or other target. In certain embodiments, the ligand is a molecule that selectively binds to a target analyte of interest (e.g., cellular marker) with high binding affinity. By high binding affinity is meant a binding affinity of at least about 10⁻⁴ M, usually at least about 10⁻⁶ M or higher, e.g., 10⁻⁹ M or higher. The ligand may be any of a variety of different types of molecules, as long as it exhibits the requisite binding affinity for the target analyte when conjugated to a fluorophore. In certain embodiments, the ligand has medium or even low affinity for its target analyte, e.g., less than about 10⁻⁴ M. As such, the ligand may be a small molecule or large molecule ligand. By small molecule ligand is meant a ligand having a size of less than 10,000 daltons, usually ranging in size from about 50 to about 5,000 daltons, and more usually from about 100 to about 1000 daltons in molecular weight. By large molecule is meant a ligand having a size of more than 10,000 daltons in molecular weight.

A small molecule ligand may be any molecule, as well as binding portion or fragment thereof, that is capable of binding with the requisite affinity to the target analyte of interest (e.g., cellular marker). Generally, the small molecule is a small organic molecule that is capable of binding to the target analyte of interest. The small molecule will include one or more functional groups necessary for structural interaction with the target analyte, e.g., groups necessary for hydrophobic, hydrophilic, electrostatic or even covalent interactions. Where the target analyte is a protein, the drug moiety will include functional groups necessary for structural interaction with proteins, such as hydrogen bonding, hydrophobic-hydrophobic interactions, electrostatic interactions, etc., and will typically include at least an amine, amide, sulfhydryl, carbonyl, hydroxyl or carboxyl group, preferably at least two of the functional chemical groups. The small molecule will also comprise a region that may be modified and/or participate in conjugation to a fluorophore, without substantially adversely affecting the small molecule's ability to bind to its target analyte.

Small molecule ligands may comprise cyclical carbon or heterocyclic structures and/or aromatic or polyaromatic structures substituted with one or more of the above functional groups. Small molecule ligands may also include organic compounds comprising alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Small molecules may include structures found among biomolecules, including peptides, carbohydrates, fatty acids, vitamins, steroids, purines, pyrimidines, derivatives, structural analogs or combinations thereof.

The small molecule may be derived from a naturally occurring or synthetic compound that may be obtained from a wide variety of sources, including libraries of synthetic or natural compounds. For example, numerous means are available for random and directed synthesis of a wide variety of organic compounds and biomolecules, including the preparation of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known small molecules may be subjected to directed or random chemical modifications, such as acylation, alkylation, esterification, amidification, etc. to produce structural analogs.

As such, the small molecule may be obtained from a library of naturally occurring or synthetic molecules, including a library of compounds produced through combinatorial means, i.e., a compound diversity combinatorial library. When obtained from such libraries, the small molecule employed will have demonstrated some desirable affinity for the protein target in a convenient binding affinity assay. Combinatorial libraries, as well as methods for the production and screening, are known in the art and described in: U.S. Pat. Nos. 5,741,713; 5,734,018; 5,731,423; 5,721,099; 5,708,153; 5,698,673; 5,688,997; 5,688,696; 5,684,711; 5,641,862; 5,639,603; 5,593,853; 5,574,656; 5,571,698; 5,565,324; 5,549,974; 5,545,568; 5,541,061; 5,525,735; 5,463,564; 5,440,016; 5,438,119; 5,223,409, the disclosures of which are herein incorporated by reference.

Small molecule ligands may also include known drugs that selectively bind to receptors on cells, including, without limitation, growth factor receptors, receptor tyrosine kinases, receptor protein serine/threonine kinases, G-protein coupled receptors, cytokine receptors, lectin receptors, and folate receptors. For example, anti-cancer drugs that bind to such cellular receptors may be used as ligands to target fluorophores to cancer cells. Exemplary drugs that may be used as ligands to target cancer cells include, without limitation, Acitinib, Afatinib, Axitinib, Erlotinib, Cabozantinib, Crizotinib, Gefitinib, Imatinib, Ibrutinib, Lapatinib, Neovastat, Nilotinib, Pazopanib, Perifosine, Ponatinib, Regorafenib, Sorafenib, Sunitinib, Trametinib, and Vandetenib.

As pointed out, the ligand can also be a large molecule. Of particular interest as large molecule ligands are antibodies, as well as antigen-binding fragments and mimetics thereof. Also suitable for use as binding agents are peptoids and aptamers. The ligand or binding agent may include a domain or moiety that can be covalently attached to a fluorophore without substantially abolishing the binding affinity for its target analyte (e.g., cellular marker).

The term “antibody” encompasses monoclonal antibodies, polyclonal antibodies, as well as hybrid antibodies, altered antibodies, chimeric antibodies, and humanized antibodies. The term antibody includes: hybrid (chimeric) antibody molecules (see, for example, Winter et al. (1991) Nature 349:293-299; and U.S. Pat. No. 4,816,567); bispecific antibodies, bispecific T cell engager antibodies (BITE), trispecific antibodies, and other multispecific antibodies (see, e.g., Fan et al. (2015) J. Hematol. Oncol. 8:130, Krishnamurthy et al. (2018) Pharmacol Ther. 185:122-134), F(ab′) 2 and F(ab) fragments; Fv molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (scFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B: 120-126); humanized antibody molecules (see, e.g., Riechmann et al. (1988) Nature 332:323-327; Verhoeyan et al. (1988) Science 239:1534-1536; and U.K. Patent Publication No. GB 2,276, 169, published 21 Sep. 1994); and, any functional fragments obtained from such molecules, wherein such fragments retain specific antigen-binding properties of the parent antibody molecule.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′) 2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety (3) single chain polypeptides containing only one heavy chain variable region, or a fragment thereof containing the three CDRs of the heavy chain variable region, without an associated light chain moiety and (4) nanobodies comprising single Ig domains from non-human species or other specific single-domain binding modules; and multispecific or multivalent structures formed from antibody fragments. In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin.

As used in this disclosure, the term “epitope” means any antigenic determinant on an antigen to which the paratope of an antibody binds. Epitopic determinants usually consist of chemically active surface groupings of molecules such as amino acids or sugar side chains and usually have specific three-dimensional structural characteristics, as well as specific charge characteristics.

“Fv” is an antibody fragment which contains an antigen-recognition and binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although often at a lower affinity than the entire binding site.

“Single-chain Fv” or “scFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains which enables the scFv to form the desired structure for antigen binding. For a review of scFv see, for example, Pluckthun, A. in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) on the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Holliger et al., (1993) Proc. Natl. Acad. Sci. USA, 90:6444-6448.

The term “affibody molecule” refers to a molecule that consists of three alpha helices with 58 amino acids and has a molar mass of about 6 kDa. A monoclonal antibody, for comparison, is 150 kDa, and a single-domain antibody, the smallest type of antigen-binding antibody fragment, 12-15 kDa. See, for exemplary details of affibody structures and uses, Orlova, A; Magnusson, M; Eriksson, TL; Nilsson, M; Larsson, B; Hoiden-Guthenberg, I; Widstrom, C; Carlsson, J et al. (2006). “Tumor imaging using a picomolar affinity HER2 binding affibody molecule”, Cancer Res. 66 (8): 4339-48. Exemplary Affibody. Molecules are commercially available from Abcam Corp. Cambridge Mass.

The phrase “specifically (or selectively) binds” with reference to binding of an antibody or other binding agent to an antigen or analyte (e.g., cellular marker such as a lesion marker, tumor-marker, or disease marker, or a vessel marker such as a lymphatic vessel marker, vein marker, or artery marker) refers to a binding reaction that is determinative of the presence of the antigen or analyte in a heterogeneous population of proteins and other biologics. Thus, under designated assay conditions, the specified antibodies or other binding agents bind to a particular antigen or analyte at at least two times the background and do not substantially bind in a significant amount to other molecules present in the sample. Specific binding to an antigen or analyte under such conditions may require an antibody or other binding agent that is selected for its specificity for a particular antigen or analyte. For example, antibodies raised to an antigen from specific species such as rat, mouse, or human can be selected to obtain only those antibodies that are specifically immunoreactive with the antigen and not with other proteins, except for polymorphic variants and alleles. This selection may be achieved by subtracting out antibodies that cross-react with molecules from other species. A variety of immunoassay formats may be used to select antibodies specifically immunoreactive with a particular antigen. For example, solid-phase ELISA immunoassays are routinely used to select antibodies specifically immunoreactive with a protein (see, e.g., Harlow & Lane. Antibodies, A Laboratory Manual (1988), for a description of immunoassay formats and conditions that can be used to determine specific immunoreactivity). Typically, a specific or selective reaction will be at least twice background signal or noise and more typically more than 10 to 100 times background.

The terms “subject”, “individual” or “patient” are used interchangeably herein and refer to a vertebrate, preferably a mammal. By “vertebrate” is meant any member of the subphylum Chordata, including, without limitation, humans and other primates, including nonhuman primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered.

### Dual Modality Imager

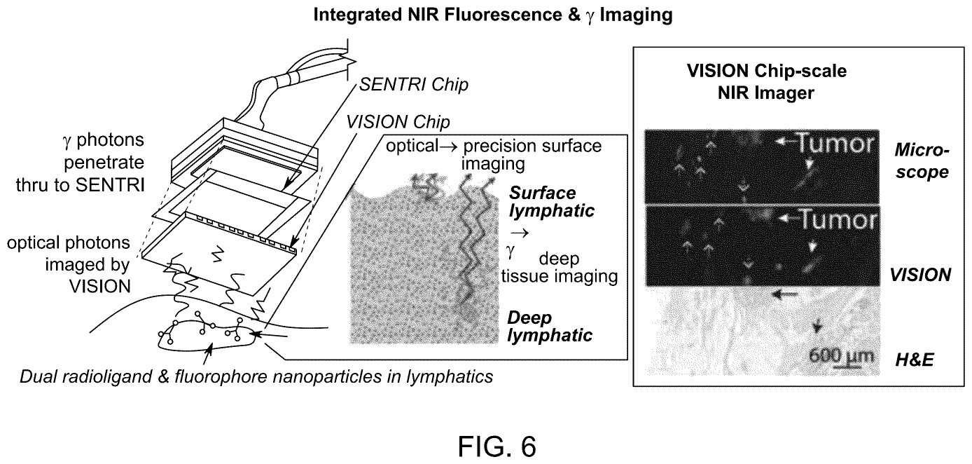

A chip-based imager capable of imaging both fluorescence and gamma photons is provided. FIG. 6 shows an exemplary dual modality imager comprising a chip-based gamma photon detector and a chip-based fluorescence detector. The chip containing the fluorescence detector is positioned relative to the chip containing the gamma photon detector such that incoming gamma photons pass through the chip containing the fluorescence detector to reach the gamma photon detector. In some embodiments, the dual modality imager is a handheld device, and further comprises a handle extending away and disposed angularly from the gamma photon detector for ease of manipulation.

In certain embodiments, the gamma photon detector component of the dual modality imager comprises an array of gamma photon-sensing pixels in a chip-based platform that integrates circuitry within each gamma photon-sensing pixel, incorporating low noise signal amplification and quantification of energy by measuring the magnitude of each signal pulse produced by an incoming gamma photon hitting a pixel. In embodiments in which the dual modality imager is used to image gamma photons from a gamma-emitting radionuclide that has been administered to a subject, in-pixel data processing may be further used to determine tissue depths of gamma-emitting radionuclide sources from which the incoming gamma photons originate. In some embodiments, the chip-based gamma photon detector is designed such that each gamma photon-sensing pixel only outputs data from the in-pixel data processing in response to an incoming gamma photon hitting the gamma photon-sensing pixel. This design obviates the need to obtain data readout of the entire chip and overcomes frame-rate limitations, which hinder large CCD-based imagers.

In certain embodiments, the fluorescence detector component of the dual modality imager comprises an array of fluorescence-sensing pixels in a chip-based platform. Miniaturization of the fluorescence detector portion of the imager is achieved, in part, by eliminating conventional optics, which is made possible because the fluorescence detector can be placed directly on tissue. The micron to millimeter-proximity of the fluorescence imager to cells allows capture of light before it diverges-preserving spatial resolution and increasing sensitivity. Therefore, difficult to miniaturize lenses are not needed. The chip-based fluorescent detector has a custom imaging array with angle-selective structures such as angle selective gratings or microcollimators for image deblurring, and optical filters that can be tuned to image fluorescence from multiple fluorophores simultaneously.

The chip-based gamma photon detectors and fluorescence detectors of the dual modality imager are described further below.

### Chip-Based Gamma-Photon Detectors

Chip-based gamma-photon detectors are provided that can be incorporated into the dual modality imager. The chip-based gamma-photon detectors use integrated circuit technology to enable gamma photon-sensing pixels to operate asynchronously, which allows determination of the energy and direction of incident gamma photons. The sensors are scalable in area, but remain thin (<300 microns). In some embodiments, the gamma photon sensors are arranged in stacks to enable energy resolution and acquire directional gamma photon information, which further enhances spatial resolution and accuracy of detection of the radionuclide distribution. The gamma sensors have minimal thickness and weight. This platform is capable of performing single gamma photon sensing for image reconstruction, analogously to SPECT, unlike positron emission tomography-based imaging, where two temporally coincident photons must be detected. Not only are no collimators needed, but rather the flux of single gamma photons is captured by an array of gamma photon-sensing pixels to reconstruct the distribution of radionuclide gamma emissions within the body. The platform also makes use of the angle of the incident gamma photon (in contrast to collimators which eliminate all angles except for a narrow window of incident angles) to back-calculate the distribution of gamma-emitting radionuclides. This approach allows for a greater number of gamma photons to be measured at a single spatial point, potentially increasing the signal to noise ratio, and speeding up the time to gather the amount of data required for accurate image reconstruction.

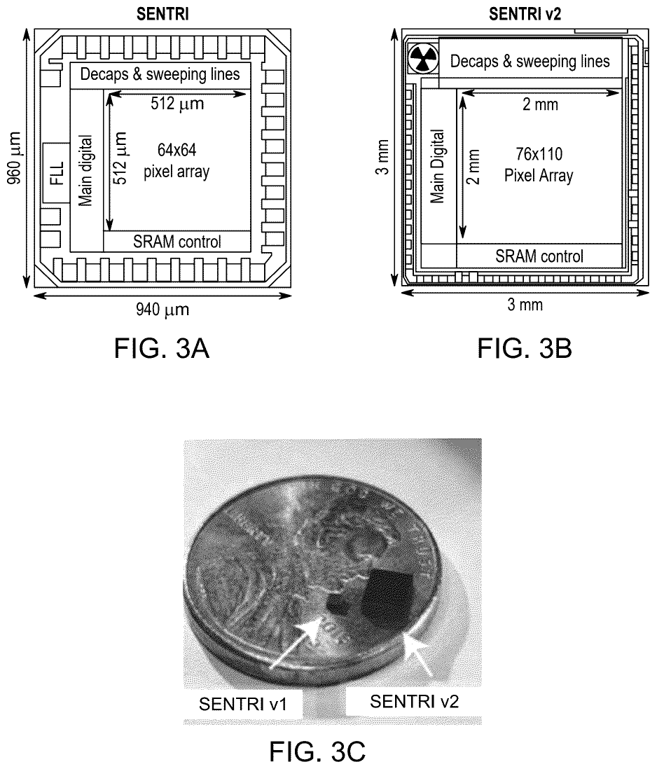

In some embodiments, each gamma photon-sensing pixel comprises at least one silicon diode, wherein gamma photons are detected by the voltage pulses they produce when they hit silicon of a diode. Without being bound by theory, the gamma photons break bonds in the silicon of the diode resulting in electron-hole pairs that generate a charge pulse in the diode, which is accumulated on a capacitor to generate the voltage pulse across the diode. In some embodiments, the chip-based gamma photon detector comprises an array of pixels, wherein each pixel is a gamma-sensing element (i.e., gamma photon-sensing pixel). FIG. 3 shows an exemplary chip-based gamma-photon detector with an array of gamma photon-sensing pixels.

The chip-based gamma photon detector may have any suitable shape such as a curved or polygonal shape. In some embodiments, the shape of the detector is circular, oval, semicircular, spherical, cylindrical, triangular, square, rectangular, pentagonal, hexagonal, octagonal, diamond-shaped, or parallelogram-shaped. In certain embodiments the detector has sides ranging from 0.1 μm to 5 mm in length, including any length within this range such as 0.1 μm, 0.2 μm, 0.3 μm, 0.4 μm, 0.5 μm, 0.6 μm, 0.7 μm, 0.8 μm, 0.9 μm, 1.0 μm, 1.5 μm, 2.0 μm, 2.5 μm, 3.0 μm, 3.5 μm, 4.0 μm, 4.5 μm, 5.0 μm, 5.5 μm, 6.0 μm, 6.5 μm, 7.0 μm, 8.5 μm, 9.0 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm 60 μm, 70 μm, 80 μm, 90 μm, 100 μm, 150 μm, 200 μm, 250 μm, 300 μm, 350 μm, 400 μm, 450 μm, 500 μm, 550 μm, 600 μm, 700 μm, 750 μm, 800 μm, 900 μm, 950 μm, 1 mm, 1.5 mm, 2 mm, 2.5 mm, 3 mm, 3.5 mm, 4 mm, 4.5 mm, or 5 mm.

In certain embodiments, the chip-based gamma photon detector has a form factor with a thickness of less than or equal to 1 cm, or less than or equal to 5 mm, or less than or equal to 3 mm, or less than or equal to 2 mm, or less than or equal to 1 mm. In some embodiments, the form factor has a thickness ranging from 1 mm to 1 cm, 1 mm to 5 mm, or 1 mm to 3 mm, including any thickness within these ranges such as 1 mm, 2 mm, 3 mm, 4 mm, 5 mm, 6 mm, 7 mm, 8 mm, 9 mm, 10 mm, 12 mm, 14 mm, 16 mm, 18 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, or 1 cm.

In certain embodiments, the chip-based gamma photon detector is implemented as an ASIC on a chip, wherein the detector comprises an array of pixels that are each gamma sensing elements. Each gamma photon-sensing pixel contains a reverse-biased silicon diode connected to a unity gain voltage buffer. Buffered voltage output from the unity gain voltage buffer is fed into a differential closed-loop amplifier. The gain of the differential closed-loop amplifier is either pre-set or configurable using in-pixel memory and DAC. A γ-photon incident on the surface of the silicon diode generates a voltage pulse across the diode and is subsequently buffered and amplified by a fixed, process-invariant gain. This voltage pulse is then digitized using a series of inverters, which are connected to digital counters to quantify the total number of gamma photons detected in a certain time frame. Each γ-photon detection event corresponds to the voltage pulse across the diode produced by each γ-photon incident on the surface of the silicon diode. The chip further comprises an on-chip data buffer configured to store a plurality of gamma photon count records for a plurality of γ-photon detection events; a digital clock configured to produce a clock signal on the chip, wherein the digital counter uses the clock signal to count numbers of γ-photon detection events per a period of time; and custom digital logic circuitry configured to control voltages and supply power from an on-chip or off-chip energy storage device to the detector, the digital counter, and the digital clock.

In certain embodiments, the chip-based gamma photon detector is implemented as an ASIC on a chip, wherein the detector comprises an array of pixels that are each gamma sensing elements, wherein each gamma photon-sensing pixel comprises two silicon diodes connected to an amplifier. An incident gamma photon breaks bonds in the silicon, generating electron-hole pairs that in turn generate a pulse of charge (Qₚ) in the silicon diode, which is accumulated on a parasitic diode capacitor (C_{diode}). This generates a small voltage pulse across the diode (Vₚ=Qₚ/C_{diode}). The use of integrated circuit technology enables ultra-small diode capacitances, increasing Vₚ, and allows for in-pixel amplification. Each voltage pulse is individually buffered using a unity gain voltage amplifier. Each of these buffered outputs connects to the inputs of a differential amplifier. To mitigate DC voltage offset at the output due to fabrication variability from pixel-to-pixel and chip-to-chip that may cause variability in detector sensitivity, a voltage integrator is bootstrapped from the output of the amplifier to one of it's inputs in a negative feedback configuration. The voltage integrator also accepts a desired DC voltage that sets the voltage at the output of the amplifier. This ensures that the sensitivity of each of the pixels across the chip is approximately the same. In order to tune each diode in each pixel to a desired sensitivity, the output of the amplifier is connected to two level shifters: one that shifts the DC level of the amplifier output up to only amplify the voltage pulse from the first diode, and the other that shifts the DC level of the amplifier output down to only amplify the voltage pulse from the second diode. The amount these DC levels are shifted are set using on-chip configurable memory and an in-pixel digital to analog converter (DAC) to convert the bits stored into an analog shift in voltage. These shifted and amplified voltage pulses are then digitized using a series of inverters. Capacitors at the inputs of the last inverters increase signal fidelity before the pulses are subsequently counted. A digital counter is coupled to the inverter chain, wherein the digital counter counts γ-photon detection events, wherein each γ-photon detection event corresponds to the voltage pulse across the diode produced by each γ-photon incident on the surface of either silicon diode. The chip also comprises an on-chip data buffer configured to store a plurality of gamma photon count records for a plurality of γ-photon detection events; a digital clock configured to produce a clock signal on the chip, wherein the digital counter uses the clock signal to count numbers of γ-photon detection events per a period of time; and custom digital logic configured to control voltages and supply power from an on-chip or off-chip energy storage device to the detector, the digital counter, and the digital clock.

In certain embodiments, the gamma photon detector is implemented as an ASIC on a chip, wherein the detector comprises an array of pixels that are each gamma sensing elements, wherein each gamma photon-sensing pixel in the array comprises silicon diodes connected to an amplifier. In some embodiments, each pixel operates asynchronously, meaning that each pixel can register a gamma interaction without reading out the entire array. This allows much greater time resolution, and enables near single particle sensitivity on the array. This feature enables determination of the incident direction of the gamma photon, which then enables improved image reconstruction with fewer incident gammas (when compared to purely counting gammas without directional information). With respect to the diodes, in some embodiments, the diodes are reversed biased. In other embodiments the diodes have zero voltage bias. With respect to the pixel architecture, in some embodiments, the pixel architecture has a differential structure, in which the two diodes are connected to the inputs of a differential amplifier. Leveraging the sparsity of photon hits, the most likely event is where only a single diode is hit at any one time, producing a differential pulse at the output of the amplifier. This structure has the advantage of mitigating offsets associated with the properties of the diode, or reset or other circuitry that will have an effect on the baseline voltage (signal) across the diode, as any mismatch between the two diodes appears as input offset of the amplifier and can cause the amplifier to operate in a low gain region, or ‘rail’ (saturate) to the point it is not active at all. Thus, the differential structure preserves the ability of the amplifier to operate with some inherent variation of the sensing diodes across the chip. The pulse from each diode is generated by an incident gamma photon breaking bonds in the silicon, which generates electron-hole pairs that in turn generate a pulse of charge (Qₚ) in the silicon diode, which is accumulated on a parasitic diode capacitor (C_{diode}). This generates a small voltage pulse across the diode (Vₚ=Qₚ/C_{diode}). The use of integrated circuit technology enables ultra-small diode capacitances, increasing Vₚ, and allows for in-pixel amplification. Each voltage pulse is individually buffered using a unity gain voltage buffer. Each of these buffered outputs connects to the inputs of a differential amplifier. In some embodiments, to mitigate DC voltage offset at the output due to fabrication variability from pixel-to-pixel and chip-to-chip that may cause variability in detector sensitivity, a voltage integrator is bootstrapped from the output of the amplifier to one of its inputs in a negative feedback configuration. The voltage integrator also accepts a desired DC voltage that sets the voltage at the output of the amplifier. This ensures that the sensitivity of each of the pixels across the chip is approximately the same. In order to tune each diode in each pixel to a desired sensitivity, the output of the amplifier is connected to two level shifters: one that shifts the DC level of the amplifier output up to only amplify the voltage pulse from the first diode, and the other that shifts the DC level of the amplifier output down to only amplify the voltage pulse from the second diode. The amounts these DC levels are shifted are set using on-chip configurable memory and an in-pixel digital to analog converter (DAC) to convert the bits stored into an analog shift in voltage. These shifted and amplified voltage pulses are then digitized using a series of inverters. Capacitors at the inputs of the last inverters increase signal fidelity before the pulses are subsequently counted. A digital counter is coupled to the inverter chain, wherein the digital counter counts γ-photon detection events, wherein each γ-photon detection event corresponds to the voltage pulse across the diode produced by each γ-photon incident on the surface of either silicon diode. The chip also comprises an on-chip data buffer configured to store a plurality of gamma photon count records for a plurality of γ-photon detection events; a digital clock configured to produce a clock signal on the chip, wherein the digital counter uses the clock signal to count numbers of γ-photon detection events per a period of time; and custom digital logic configured to control voltages and supply power from an on-chip or off-chip energy storage device to the array of gamma photon-sensing pixels, the digital counter, and the digital clock.

In certain embodiments, the chip is covered with a material capable of Compton scattering of gamma photons. The Compton scattering material may include, but is not limited to, lead, tungsten, or bismuth. Compton scattering of gamma photons that collide with the material generates lower energy photons (typically <100 keV) that have a higher interaction probability with the detector due to the photoelectric effect and additionally creates secondary electrons from the inelastic collision with the primary gamma photon, which have a high interaction probability with the detector. In some embodiments, these secondary electrons generated from the gamma photons are detected by the detector. Both of these energy conversions can be accomplished using a thin high-density layer of a Compton scattering material on top of the detector or on the back of the detector (e.g., covering the bulk silicon), which can boost the signal of the detector, increase its sensitivity, and minimize data acquisition times.

In certain embodiments the energy of the incoming photons can be determined by measuring the ratio of detector counts with a plurality of detectors having varying thicknesses of attenuating material. The attenuating material may include lead, tungsten, bismuth, or other high-density material. For example, lower energy photons have an exponentially higher probability of interaction with lead, and can be detected without or in certain instances with a very thin layer of lead. Higher energy photons are selectively measured by creating a subsequently thicker layer of lead on top of other detectors to completely attenuate lower energy photons and only allow high energy photons to pass through. This process can be repeated with layers of different thicknesses in order to allow for energy resolution of an incoming gamma photon flux consisting of multiple primary energy emissions to be determined. Since the energies of gamma photons will be a distribution at varying thicknesses of lead, an understanding of the likelihood of detecting a photon of a certain energy with a detector of a certain lead thickness can be determined using multiphysics simulation, and can be used to separate the detected counts of each energy.

In certain embodiments, chip-based gamma detectors, capable of energy resolution and/or incident direction resolution of an incoming flux of gamma photons, are incorporated into the dual modality imager. In some embodiments, the chip-based gamma detectors are placed in a vertical stack with each detector separated from the other with a thin layer of an attenuating material, which may include lead, tungsten, bismuth, or other high-density material.

In certain embodiments, chip-based gamma detectors are stacked on top of each other (i.e., to create a gamma sensor stack), which enables incident angle measurements of incoming photons. In some embodiments, the stacked chip-based gamma detectors are each equipped with fast readout circuitry for their array of pixels (i.e., gamma sensing elements) to allow for near instantaneous detection of the same incident gamma photon passing between two chip-based gamma detectors in the stack. In some embodiments, this near instantaneous detection of incident gamma photons is accomplished by having asynchronous pixel operation, such that each pixel samples the gamma photon, and transmits the time that the gamma photon hits the detector, the pixel location on the detector, and the signal, which is related to the LET of the gamma detector. The angle shift of the gamma photon passing from the chip-based gamma detector at the top of the stack to an underlying chip-based gamma detector can be used to calculate the incident gamma photon angle on the gamma sensor stack by evaluating the numerical equations that govern Compton scattering physics.

In some embodiments, the chip-based gamma detectors are stacked with a stack thickness of greater than or equal to 3 mm and less than 5 mm. In some embodiments, chip-based gamma detectors are stacked with a stack thickness of greater than or equal to 1 mm and less than 3 mm. In some embodiments, chip-based gamma detectors are stacked with a stack thickness of greater than or equal to 0.1 mm and less than 1 mm.

In some embodiments, the chip-based gamma detectors are stacked with a form factor having a thickness of less than or equal to 1 cm, or less than or equal to 5 mm, or less than or equal to 3 mm, or less than or equal to 2 mm, or less than or equal to 1 mm. In some embodiments, the form factor has a thickness ranging from 1 mm to 1 cm, 1 mm to 5 mm, or 1 mm to 3 mm, including any thickness within these ranges such as 1 mm, 2 mm, 3 mm, 4 mm, 5 mm, 6 mm, 7 mm, 8 mm, 9 mm, 10 mm, 12 mm, 14 mm, 16 mm, 18 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, or 1 cm.

In certain embodiments, chip-based gamma detectors that are capable of determining the energy resolution of an incoming flux of gamma photons are placed in the same plane to give energy resolution in approximately the same spatial area. In some embodiments, each detector has varying thickness of an attenuating material such as lead, tungsten, bismuth, or other high-density material placed on their surface or above the bulk silicon.

In certain embodiments, on-chip circuitry is tuned to be responsive to a range of linear energy transfer (LET), such that the combination and distribution of signals from various pixels with known LET responsivity enable the determination of the incident energies of incoming gamma photons. For example, low energy photons have higher LET and can be detected with a pixel with low gain that only detects low energy photons and not higher energy photons with higher LET, which do not produce a signal. On the same chip or a neighboring chip, pixels with higher gain are included to amplify the low LET from higher energy photons. The pixels with higher gain are responsive to both lower energy photons and higher energy photons. By combining the statistics from sets of pixels with lower gains and sets of pixels with higher gains, the energy distribution of incoming gamma photons can be determined.

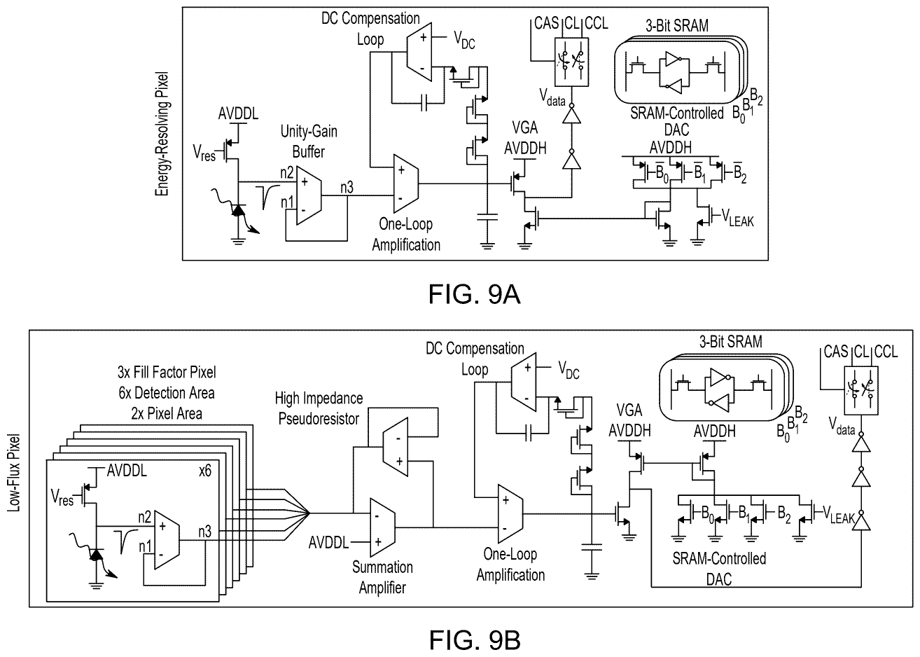

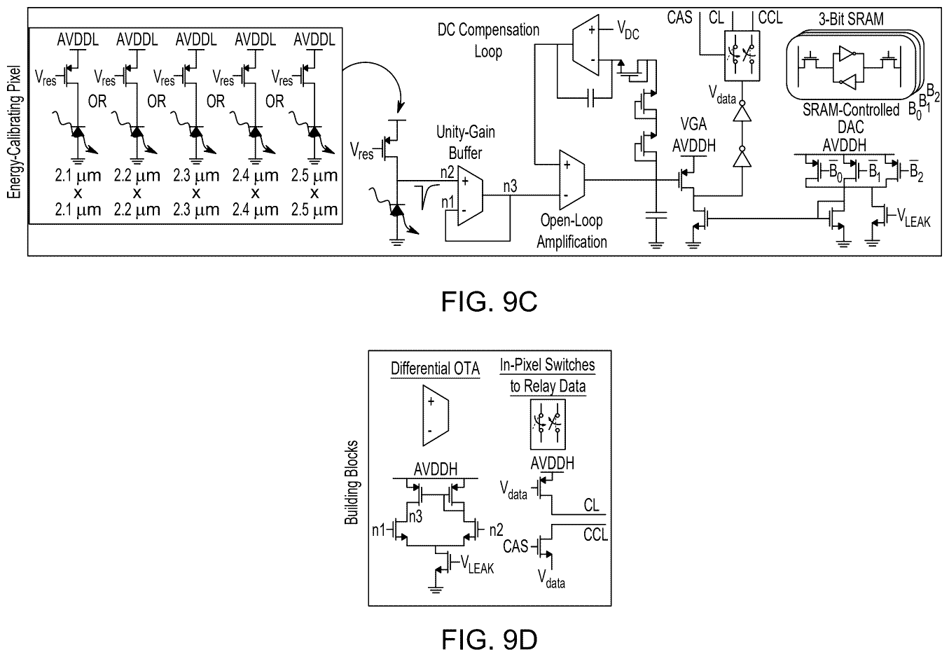

In an exemplary embodiment illustrated in FIG. 9, the array of gamma photon-sensing pixels comprises a plurality of energy-resolving pixels, a plurality of low-flux pixels, and a plurality of energy-calibrating pixels. Each energy-resolving pixel comprises a reverse-biased diode that generates a voltage drop at a diode node in response to energy deposition by an incoming gamma photon hitting the reverse-biased diode, wherein the reverse-biased diode is connected to a unity-gain buffer, wherein output of the unity-gain buffer is connected to an operational transconductance amplifier (OTA) with a direct current (DC) compensation loop that secures an output DC level of the OTA, wherein output of the OTA is routed to a p-channel metal-oxide-semiconductor (PMOS) common-source (CS) stage with a configurable load to set a custom sensitivity of the energy-resolving pixel, wherein the load is configured with a 3-bit in-pixel six transistor (6T) static random-access memory (SRAM) that is used to control in-pixel digital-to-analog converters (DACs) that set a gate voltage of the load; and two inverters, wherein voltage pulses are digitized using the two inverters and sent to a digital processing unit via a set of switches, wherein widths of the digital pulses are measured to estimate a voltage signal decay time (DT). Each low-flux pixel comprises 6 reverse-biased diodes, wherein each reverse-biased diode is coupled to a unity-gain buffer, wherein buffer outputs from each unity-gain buffer are connected together and routed to an OTA comprising a unity-gain buffer in a feedback loop, said OTA serving as a pseudoresistor to create a summing amplifier with gain=−1, wherein summation of individual gamma photon hits on any of the 6 reverse-biased diodes is routed to an open-loop OTA comprising a DC compensation loop, N-type metal-oxide-semiconductor (NMOS)CS-stage with an SRAM-configurable load, and inverters for count digitization. The energy-calibrating pixels comprise a plurality of reverse-biased diodes of varying diode sizes having different diode parasitic capacitances and different voltage drops in response to energy deposition by an incoming gamma photon, wherein the plurality of reverse-biased diodes are connected to a unity-gain buffer, wherein output of the unity-gain buffer is connected to an OTA with a DC compensation loop that secures an output DC level of the OTA, wherein output of the OTA is routed to a PMOS CS stage with a configurable load to set a custom sensitivity of the energy-calibrating pixel, wherein the load is configured with a 3-bit in-pixel 6T SRAM that is used to control in-pixel DACs that set a gate voltage of the load; and two inverters, wherein voltage pulses are digitized using the two inverters and sent to a digital processing unit via a set of switches, wherein the energy-calibrating pixels are used for energy-binning to determine the maximum energy deposited (E_{dep}) by a gamma photon, wherein a least significant bit (LSB) is selected to optimize energy resolution and dynamic range, and wherein the LSB is used to estimate the DT. In certain embodiments, the varying diode sizes of the energy-calibrating pixels have diode areas of 4.41 μm², 4.84 μm², 5.29 μm², 5.76 μm², and 6.25 μm².

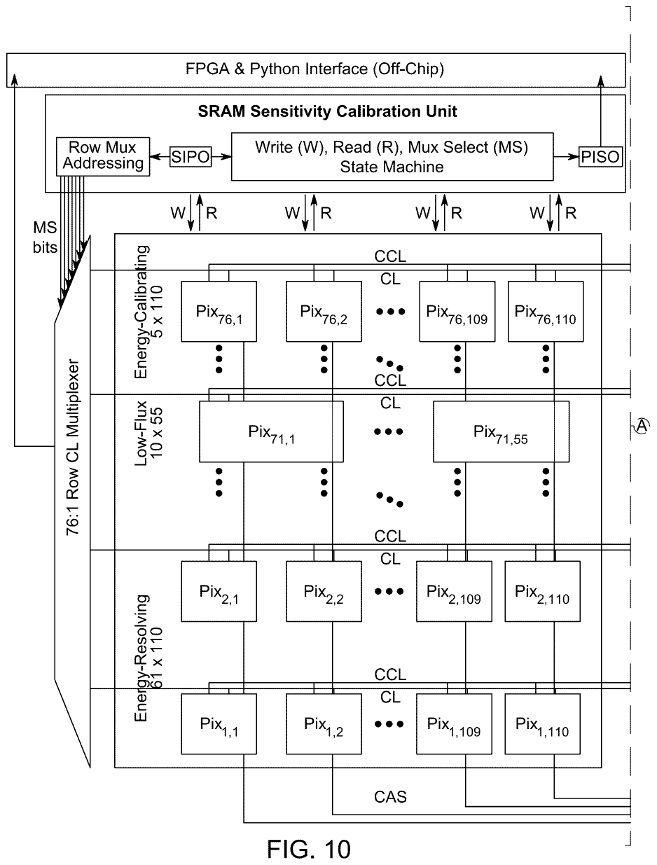

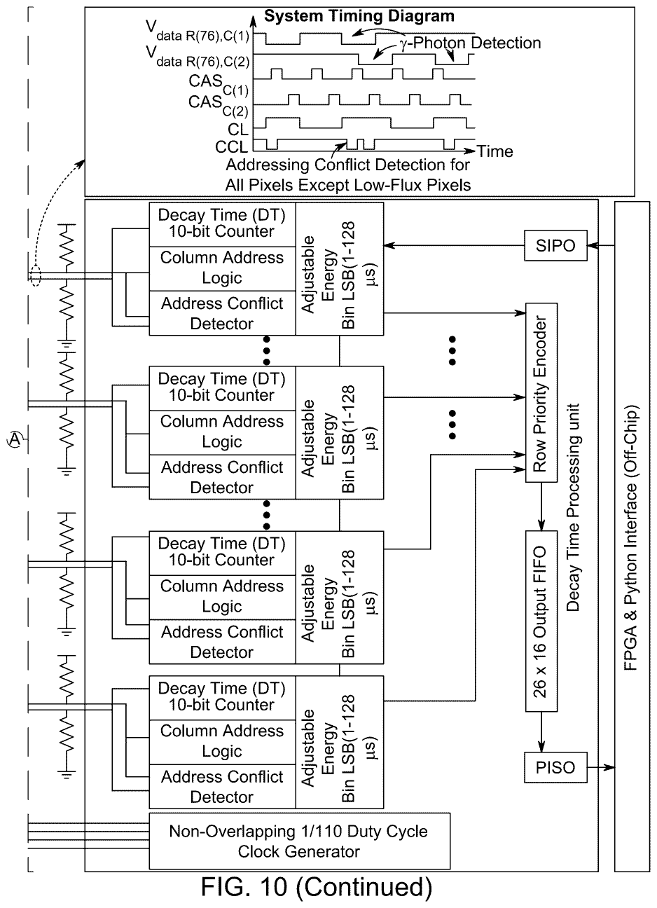

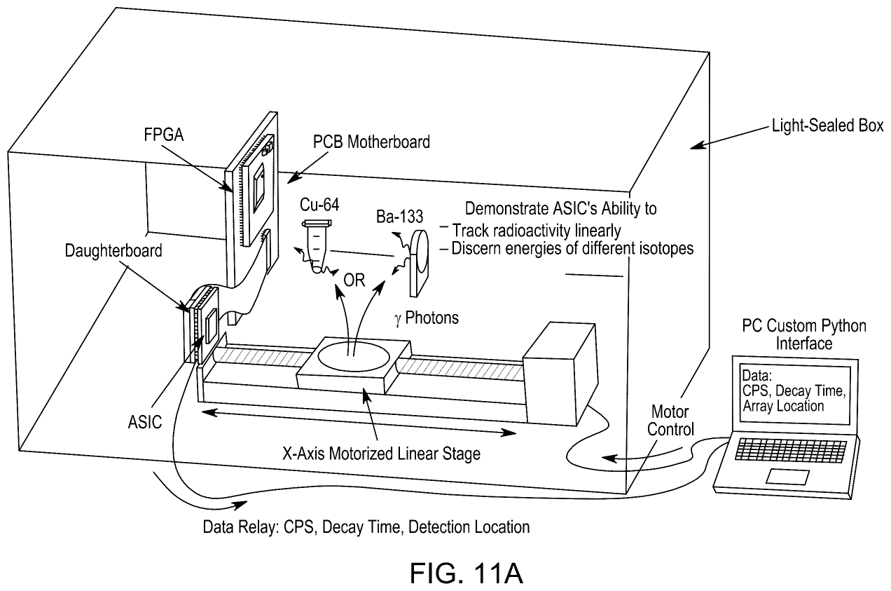

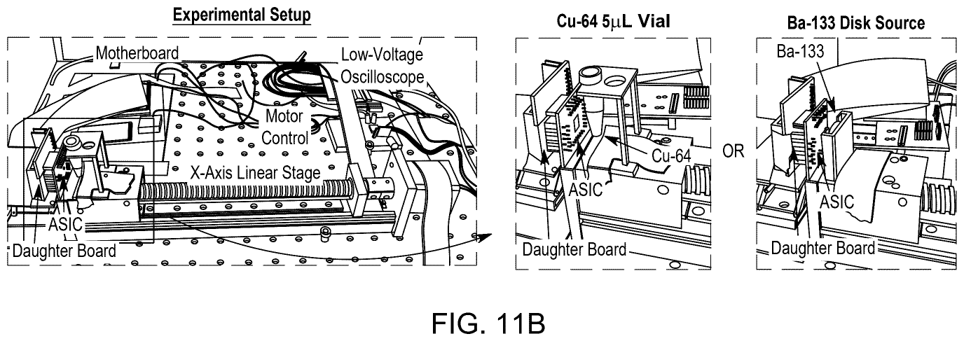

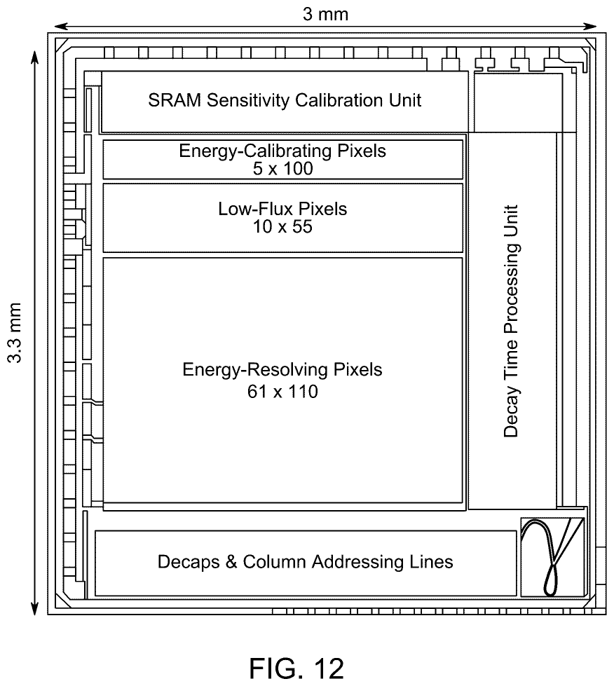

In another exemplary embodiment, illustrated in FIG. 10, the array of gamma photon-sensing pixels comprises a plurality of rows of energy-resolving pixels, a plurality of rows of low-flux pixels, and a plurality of rows of energy-calibrating pixels, wherein each row in the array of gamma photon-sensing pixels shares a count-line (CL) and a count column line (CCL), wherein the CL outputs a pulse whenever a gamma photon is detected by a gamma photon-sensing pixel, wherein numbers of pulses output by the CL are measured with a 10-bit counter, wherein non-overlapping column address sweep (CAS) signals are routed down every column of the array of gamma photon-sensing pixels such that when a gamma photon is detected, the CCL outputs a pulse to identify the column address, wherein the DT, column, and row data are priority encoded, queued into a first in first out (FIFO) memory element, serialized, and sent to a field programmable gate array (FPGA).

As further illustrated in FIG. 10, the dual modality imager may also comprise an SRAM sensitivity calibration unit, wherein the SRAM sensitivity calibration unit is used to read or write to a 3-bit in-pixel SRAM to control the gain of each CS-stage. Each gamma photon-sensing pixel can be initially calibrated in the absence of gamma radiation. To set a maximum gain threshold, SRAM values are increased in order to increase the CS gain until the CL is triggered by noise (i.e., in the absence of detecting a gamma photon). The maximum gain threshold is set at a level just below the level at which the CL is triggered by noise.

In certain embodiments, a combination of two or more or all the above methods for energy resolution are used to determine the energy distribution of incoming gamma photons.

In certain embodiments, a gamma photon-sensing pixel is also responsive to electrons generated by the gamma photons. Electrons may be generated by gamma photons by the photoelectric effect, wherein a gamma ray transfers all its energy to an electron resulting in ejection of the electron from an atom. Alternatively, electrons may be generated from gamma photons by Compton scattering, which similarly results in ejection of an electron from an atom, wherein the gamma ray retains some of its energy and is scattered in a different direction. In some cases, electrons are generated from gamma photons in a patient administered a radiopharmaceutical comprising a gamma-emitting radionuclide, in a layer of material placed in between the patient and a pixel sensor, or in silicon of a diode upon collision with a gamma photon. Electrons bombarding a silicon diode create electron-hole pairs in the silicon that generate voltage pulses across the silicon diode, which can be detected similarly to those produced by gamma photons with the chip-based devices described herein.

The number of diodes contained in a pixel array (e.g., for detection of gamma photons or electrons generated by gamma photons) may vary. In certain embodiments, a pixel array includes two or more diodes, such as 10 or more, 50 or more, 100 or more, 500 or more, 1000 or more, 2000 or more, 3000 or more, or 4000 or more, including 5000 or more, e.g., about 2 to 10 diodes, about 10 to 100 diodes, about 100 to 500 diodes, about 500 to 1000 diodes, about 1000 to 2000 diodes, about 2000 to 3000 diodes, about 3000 to 4000 diodes, or about 4000 to 5000 diodes, including any number of diodes in these ranges such as 2, 4, 6, 8, 10, 20, 40, 60, 80, 100, 120, 140, 160, 180, 200, 250, 300, 350, 400, 450, 500, 550, 600, 650, 700, 750, 800, 850, 900, 950, 1000, 1500, 2000, 2500, 3000, 3500, 4000, 4500, or 5000 diodes. In one embodiment, the pixel array comprises 4,096 diodes. The diodes may be arranged in a regular repeating pattern (e.g., a grid, such as a grid with about 1 μm to 10 μm spacing between diodes), or no pattern. Ultra-small micron-scale diodes having high sensitivity for detection of single gamma photons can be used in such arrays. Micron-scale photodiodes are commercially available, for example, from X-FAB Silicon Foundries (Erfurt, Germany) and Taiwan Semiconductor Manufacturing Company (Hsinchu Science Park, Taiwan). Such diodes can be used in massively parallel arrays for detection of gamma photons with high sensitivity. In some embodiments, thousands of such micron-scale gamma ray-sensitive photodiodes are included in pixel arrays on a millimeter-scale chip.

In some embodiments, the pixel array is optimized for sensitivity by decreasing the diode capacitance. In some embodiments, the diode size is the minimum size available for the CMOS process (which is continually decreasing). In some embodiments, the diode size is 0.1 μm to 0.5 μm×0.1 μm to 0.5 μm. In some embodiments, the diode size is 0.5 μm to 1 μm×0.5 μm to 1 μm. In some embodiments, varying diode sizes are used in an array of gamma photon-sensing pixels, including diodes having an area of 3 μm² to 7 μm² such as, but not limited to diodes having an area of 4.41 μm², 4.84 μm², 5.29 μm², 5.76 μm², and 6.25 μm².

In some embodiments, the pixel array is optimized to provide a tradeoff between both pixel fill factor and sensitivity by setting the diode size to 1 μm to 1.5 μm×1 μm to 1.5 μm.

In certain embodiments, the detector is optimized to increase fill factor with a tradeoff in sensitivity by setting the diode size to 1.5 μm to 3 μm×1.5 μm to 3 μm.

In certain embodiments, the detector is optimized to increase fill factor with a tradeoff in sensitivity by setting the diode size to 3 μm to 10 μm×3 μm to 10 μm.

In certain embodiments, the detector is optimized to increase fill factor with a tradeoff in sensitivity by setting the diode size to 10 μm to 50 μm×10 μm to 50 μm.

In some embodiments, the diodes are connected to an amplifier to improve sensitivity of detection of gamma photons. For a description of suitable amplifiers and amplification circuitry, see, e.g., Lee et al. (2020) Int J Part Ther 6 (3): 35-109; Lee et al. (2020) A 64×64 Implantable Real-Time Single-Charged-Particle Radiation Detector for Cancer Therapy, IEEE International Solid-State Circuits Conference-(ISSCC), IEEE p. 506-508 (ieeexplore.ieee.org/document/9063125); herein incorporated by reference in their entireties.

In some embodiments, a digital counter is used to count the number of γ-photon detection events, i.e., the voltage pulses produced by a γ-photon incident on the surface of a silicon diode. In certain embodiments, the chip-based gamma photon detector further comprises a clock configured to produce a clock signal representing time, wherein the digital counter uses the clock signal to count numbers of γ-photon detection events per a set period of time (e.g., counts per second (CPS)). In some embodiments, the clock signal is generated by a frequency locked loop (FLL) oscillator (see, e.g., FIG. 31). A clock beacon to the FLL may be generated from an off-chip crystal oscillator. In other embodiments, the clock signal is generated directly from an off-chip crystal oscillator. In certain embodiments the clock signal and control signals are generated from an external computer, FPGA, cellular phone, or other control device.

The digital output of the digital counter can be stored in an internal data storage unit (memory) or an external data storage unit in communication with or connected to the digital counter, either with a wire or wirelessly. The data storage unit can be configured to store a plurality of gamma photon count records for a plurality of γ-photon detection events. The data storage component may be of any type capable of storing information, and may utilize, e.g., FLASH memory, metal-oxide-semiconductor (MOS) memory, random-access memory (RAM), dynamic random-access memory (DRAM), static random-access memory (SRAM), synchronous dynamic random-access memory (SDRAM), or any other write-capable memory. In certain embodiments, the gamma photon detector comprises on-chip memory in the form of static random-access memory (SRAM).

In certain embodiments, the chip-based gamma-photon detector further comprises a first wireless communication unit in communication with the internal data storage unit (memory) and an external data receiving device comprising a second wireless communication unit. In some embodiments, the first wireless communication unit utilizes a wireless communication protocol using an electromagnetic carrier wave (e.g., radio wave, microwave, or infrared) or ultrasound to transfer data from the data storage unit to the external data receiving device comprising the second wireless communication unit. For example, the first wireless communication unit may utilize a radio-frequency communication protocol or an ultrasound communication protocol to transfer data from the data storage unit to the external data receiving device comprising the second wireless communication unit. The data receiving device may include, without limitation, a computer or handheld device, such as a cell phone or tablet. In certain embodiments the data is uploaded wirelessly to the cloud.

In some embodiments, the chip-based gamma-photon detector further comprises an edge computing device connected to the internal data storage unit (memory), wherein the edge computing device receives gamma photon count data. In some embodiments, the on-chip edge computing device is programmed to partially process the count data, which is subsequently transmitted to an external data processing unit to complete data processing. In certain embodiments, the data storage unit is in communication with an external data processing unit, wherein the data processing unit is programmed to calculate total % IA/ml for one or more tumors and one or more organs at risk in a subject from a plurality of gamma photon count records from multiple gamma photon detectors, as described further below.

In some embodiments, the chip comprises an internal power source or energy storage device such as a capacitor or battery to supply power to the chip, for example, for operation of the digital counter, detector, and clock. The on-chip power source or energy storage device can include, without limitation, lithium ion batteries, silver oxide batteries, or chip-type electric double layer capacitors. In some embodiments, the battery is rechargeable.

In some embodiments, power is transmitted to the chip from an external transducer. Examples of external transducers include an ultrasound transducer, an electromagnetic (EM) transducer, an inductive transducer, and a radiofrequency (RF) transducer, among others. In certain embodiments, the chip further comprises an energy storage device such as a capacitor or a rechargeable battery to store electrical energy output from a piezoelectric substrate in response to receiving ultrasound power or an antenna in response to receiving RF power or electromagnetic power, wherein the capacitor or rechargeable battery supplies power to the chip for operation of the chip. In some embodiments a piezoelectric substrate and/or battery or storage capacitor are assembled on a solid support (e.g., board) containing a chip. In certain embodiments, the gamma photon detector comprises a precharged battery, and does not receive external power or recharge, in which case, after discharging, the battery remains until it is removed.

Several voltage regulators may be used to generate the on-chip voltages for circuit operation and on-chip clock generation. In embodiments where power is transferred from an external transducer, the gamma photon detector further comprises a voltage rectifier and several voltage regulators to generate the on-chip voltages for circuit operation. Multiple voltage sources at various levels can be generated on-chip to power circuitry on the chip. In some embodiments, the clock signal is derived from a transducer signal. An analog to digital converter (ADC) can also be included on the chip. Examples of an ADC include an 8-bit differential SAR ADC with a 0.5 V or 1 V maximum range and a 10-bit SAR ADC with a 0.5 V or 1 V maximum range, among others.

For a further description of chip-based gamma photon detectors, which can be incorporated into the dual modality imager, see also International Patent Application Publication No. WO 2025/059557 A1; herein incorporated by reference in its entirety.

### Chip-Based Fluorescence Detectors

Chip-based fluorescence detectors are also provided that can be incorporated into the dual modality imager. In some embodiments, the chip-based fluorescence detector comprises an array of fluorescence sensing pixels with angle-selective structures such as angle selective gratings or microcollimators for image deblurring, and optical filters that can be tuned to image fluorescence from multiple fluorophores simultaneously. Power is supplied by an on-chip power source or transmitted to the chip from an external transducer such as, but not limited to, an ultrasound transducer, an electromagnetic (EM) transducer, an inductive transducer, or a radiofrequency (RF) transducer. For example, an ultrasound transducer can be used to supply power wirelessly to the imager through a piezoelectric substrate (e.g., piezoelectric crystal or ceramic) incorporated into the integrated circuit. Alternatively, the imager may further comprise an on-chip antennae configured to receive radiofrequency (RF) power from an external RF transducer or electromagnetic power from an EM or inductive transducer. Power supplied by an external transducer can be used to power the chip operation directly or stored in a capacitor for later use. In some embodiments, power from an external transducer is used to charge a rechargeable on-chip battery, which can be used to power chip operations. Data can be wirelessly transmitted or stored and then transmitted.

The chip-based fluorescence detector comprises a plurality of photodiodes arrayed on the surface of the chip (i.e., array of fluorescence-sensing pixels), wherein each photodiode is coated with a layer of material (or multiple materials) that functions as an optical filter (e.g., an absorption filter or an interference filter). In certain embodiments, multiple layers of filter material are used in combination with micro-collimators to block illumination light and allow fluorescence light to pass through to the photodiodes.

In certain embodiments, the optical filter comprises an absorption filter having a band gap and thickness suitable to allow light at a fluorescence emission wavelength of a fluorophore of interest to pass through to the photodiode. Exemplary absorption filter materials include, without limitation, amorphous silicon, crystalline silicon, gallium phosphide, cadmium selenide, gallium arsenide, and indium phosphide. Different fluorophores with different emission spectra (colors) can be imaged by using absorption filter materials with different band gaps. In addition, the absorption filters in the array can be tuned by varying the thickness of the layer of absorption filter material on individual photodiodes. In certain embodiments, the band gap and/or thickness of the layer of absorption filter material is varied on a plurality of photodiodes in the array to allow selection of light at different fluorescence emission wavelengths to allow fluorescence imaging of multiple fluorophores having different fluorescence emission spectra. In some embodiments, the thickness of the layer of absorption filter material on at least two photodiodes is different. In other embodiments, the thickness of the layer of absorption filter material on all the photodiodes is the same.

In certain embodiments, the thickness of the layer of absorption filter material on a photodiode is less than 100 microns. For example, the thickness of the layer of absorption filter material may range from 1 micron to 100 microns, including any thickness within this range, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 microns in thickness. In some embodiments, the thickness of the layer of absorption filter material ranges from about 5 microns to about 30 microns

In certain embodiments, the optical filter is an interference filter. In some embodiments, the interference filter is a single bandpass, dual bandpass, triple bandpass, or quadruple bandpass interference filter. In some embodiments, the interference filter further comprises a layer of absorption filter material on top of the interference filter or underneath the interference filter.

In certain embodiments, the interference filter further comprises one or more layers of material comprising a plurality of angle selective gratings, collimators, or fiber optic plates that block light that is not incident within 30° of an axis perpendicular to the plane of the chip. In some embodiments, fiber optic plates are used that block light that is not incident within 6° of an axis perpendicular to the plane of the chip (e.g., having a FWHM of 12 degrees). In some embodiments, angle selective gratings are used that block light that is not incident within 15° of an axis perpendicular to the plane of the chip.

In some embodiments, one or more layers of material comprising the plurality of angle selective gratings, collimators, or fiber optics is on top of the layer of filter material (between the filter and cells), underneath the layer of filter material (between the filter and photodiodes), or both on top and underneath the layer of filter material. In some embodiments, the angle selective gratings, collimators, or fiber optic plate are in a layer of material on top of the layer of filter material and block light that is not incident within 10°-15° of an axis perpendicular to the plane of the chip. In some embodiments, this layer on top of the layer of filter material has a thickness of about 100 microns to about 500 microns. In some embodiments, the angle selective gratings, collimators, or fiber optics are in a layer of material underneath the layer of filter material and block light that is not incident within 5°-30° of an axis perpendicular to the plane of the chip. In some embodiments, this layer underneath the layer of filter material has a thickness of about 8 microns or less. Surrounding the collimators or fiber optics may be a material that is opaque to the incident light such that only light passing through the collimators or fiber optics may pass through the filter. This ensures proper operation of interference-based filters which have an angle-dependent response, and whose performance decays with oblique angles of incidence. In certain embodiments, the interference filter may be deposited on either one or both surfaces of the fiber optic plate or directly on the chip. The interference filter may have any of the following filter characteristics: long-pass, short-pass, notch, bandpass, or a combination of any number of these characteristics.

In some embodiments, the optical filter comprises an interference filter with an array of micro-collimators (or micro-channels) affixed to the “top” surface (between the filter and cells), or the “bottom” surface (between the filter and photodiodes), or both. The purpose of the layer of collimators is to eliminate light incident at oblique (“off-axis” or non-perpendicular) angles, that may bleed through the interference filter. The collimator array blocks light at oblique angles (for example>30 degrees of the perpendicular axis) while allowing light that is incident perpendicularly (for example from 0-10 degrees) to pass through. The microcollimators can be made of transparent material separated by a cladding or material that is either opaque, or of a higher index of refraction such that light is trapped in the collimator and the critical angle is less than 30 degrees. Light>30 degrees can be extinguished by at least 1, 2, 3, 4, 5, or 6 orders of magnitude relative to light entering at <10 degrees). Alternatively, a fiber optic array or fiber optic plate can be used on top of the interference filter layer to reject fluorescent light emitted incident at oblique/off perpendicular angles that will not be adequately rejected by the filter-since it has an angle dependent response. In addition, angle selective gratings can be placed between the filter and the imaging chip. An additional layer of absorption filter can be affixed either on top of the filter or underneath the filter to absorb any illumination light that may penetrate through the interference filters when light incidence is off axis. The interference filter may include, without limitation, a single bandpass, dual bandpass, triple bandpass, or quadruple bandpass filter to allow for multicolor imaging of tissue using the same image sensor.

A light emitting source is used to excite fluorescent emissions from a fluorophore of an imaging agent. Any suitable light source may be used for fluorescence excitation. Exemplary light sources include, without limitation, halogen lamps, light-emitting diodes (LEDs), and laser diodes. The light source may be located on the dual modality imager (e.g., on the chip-based fluorescent detector or elsewhere) or externally. In addition, band-pass filters can be used to produce multiple excitation channels from a light source and narrow the band of excitation. In some embodiments, a light emitting source is located on a chip. Light emitting sources that can be used on a chip include, without limitation, micro-laser diodes and LEDs. In some embodiments, LEDs are used with emission filters. Alternatively, LEDs having a tight enough full-width half maximum spread can be used without emission filters. The light sources may emit light of a single color (wavelength) or multiple colors (wavelengths) so as to illuminate multiple fluorophores. Multiple light sources of the same and/or different wavelength emissions may be arrayed on a chip, and used in parallel or serially to illuminate tissue. In some embodiments, an external light emitting source is on a separate chip from the chip-based fluorescence detector. In some embodiments, a chip comprising an external light emitting source is implantable in tissue. The internal and/or external light sources can be activated together or sequentially.

In certain embodiments, a plurality of light emitting sources are located externally from the chip-based fluorescence detector of the dual modality imager. In some embodiments, the external light emitting source is on a separate chip, which may be placed in the dual modality imager or externally. In some embodiments, the external light emitting source is implantable in tissue. For example, a “micro-star” external light source can be used to illuminate tissue having target cells labeled with a fluorescent imaging agent (see International Patent Application Publication No. WO 2022/165234 A1, herein incorporated by reference in its entirety). A micro-star comprises a chip similar to that used for a chip-based fluorescence detector except that the chip does not have an array of fluorescence-sensing pixels with photodiodes and does not transmit imaging data. The primary purpose of a micro-star is to illuminate tissue. The micro-star chip may comprise, for example, a single wavelength, laser diode; two laser diodes of different wavelengths; three laser diodes of different wavelengths; etc. Alternatively, the micro-star chip may comprise one or more LEDs instead of laser diodes. LEDs tend to be more power efficient than laser diodes, but have a broader wavelength spread. Therefore, emission filters may be used with the LEDs to prevent wavelengths of light from being in the passband of the corresponding optical filter. The wavelength of the light emitted from each laser diode or LED should overlap the excitation wavelength of an intended fluorophore and not overlap with the emission wavelengths of simultaneously imaged fluorophores or fall into the any of the emission pass bands of the filter. The same arrangement of LEDs or laser diodes that is used on a micro-star can also be incorporated onto a corresponding chip comprising a fluorescence detector. Similarly to the chip-based fluorescence detector, power can be supplied to the micro-star by an on-chip power source or transmitted to the chip from an external transducer. In some embodiments, the micro-star chip comprises an on chip-energy storage device (e.g., battery or capacitor) to supply power for operation of one or more light-emitting sources on the chip. An on-chip driver circuit will provide power to the light-emitting sources. Alternatively the driver circuit may be located off chip. This driver may be capable of supplying a wide range of bias currents, may be tolerant of a wide range of required on voltages of the light sources, and can be configurable to the each of the above ends. The micro-star chip may further comprise a piezoelectric substrate (e.g., piezoelectric crystal or ceramic) incorporated into the integrated circuit to receive power from an external transducer, such as an ultrasound transducer. In some embodiments, the same ultrasound transducer that is used to supply power wirelessly to the chip-based fluorescence detector is used to supply power to the micro-star to turn on the light emitting sources on the micro-star to provide illumination. In some embodiments the platform comprises a network of sensors comprising multiple chip-based fluorescence detectors integrated with light emitting sources, and multiple independent light sources (e.g., micro-stars). Each mote is powered by an on chip-energy storage device: either a battery or energy storing capacitor which is charged up from an external power source (such as ultrasound transducer or RF transducer). In the case of ultrasound power transfer, an ultrasound transducer is used to supply power wirelessly through a piezoelectric crystal/ceramic incorporated into the integrated circuits of the imager and/or micro-stars.

In certain embodiments, the chip-based fluorescence detector comprises an on-chip power source or energy storage device such as a capacitor or battery (for example measuring<5 mm3) to supply power for operation of the chip-based fluorescence detector. The on-chip power source or energy storage device can include, without limitation, lithium ion batteries, silver oxide batteries, or chip-type electric double layer capacitors. In one embodiment, the battery has dimensions of 3.6 mm×5.5 mm×1 mm with an energy capacity of 300 uA hours. In some embodiments, the battery is rechargeable. In some embodiments, a battery and a capacitor are used to store the levels of power needed to operate the chip.

In other embodiments, the on-chip power source comprises radionuclides, from which power is harvested directly or indirectly. For example, power can be harvested from radionuclides through direct interaction of emitted radionuclides with on-chip diodes, or indirectly through conversion of emitted radionuclide particles (such as an electron or alpha particle) to light via a scintillating material, and then captured by a photovoltaic. The power derived from the radionuclides can be used to charge a battery or capacitor, or power the system directly. In some embodiments, the chip-based fluorescence detector comprises an on-chip photovoltaic system or a nuclear battery or a radionuclide in combination with a scintillator and photovoltaic energy harvester to provide power to the chip-based fluorescence detector chip.

In some embodiments, power is transmitted to the chip from an external transducer such as, but not limited to, an ultrasound transducer, an electromagnetic (EM) transducer, an inductive transducer, or a radiofrequency (RF) transducer.

In some embodiments, the chip-based fluorescence detector further comprises a piezoelectric substrate that generates piezoelectricity in response to exposure to ultrasound from an external ultrasound transducer. Any suitable piezoelectric crystal or ceramic may be used in the imager. Exemplary piezoelectric crystals include, without limitation, langasite (La₃Ga₅SiO₁₄), gallium orthophosphate (GaPO₄), lithium niobate (LiNbO₃), lithium tantalate (LiTaO₃), quartz, berlinite (AlPO₄), Rochelle salt, topaz, tourmaline-group minerals, and lead titanate (PbTiO₃). Exemplary piezoelectric ceramics include, without limitation, ceramics with perovskite, tungsten-bronze, lead zirconate titanate (Pb[ZrₓTi₁₋ₓ]O₃, potassium niobate (KNbO₃), sodium tungstate (Na₂WO₃), Ba₂NaNb₅O₅, Pb₂KNb₅O₁₅, and zinc oxide (ZnO). In certain embodiments that the piezoelectric ceramic/crystal is made of toxic material, a biocompatible layer of Parylene C is used to encapsulate the piezoceramic without impacting its power harvesting and data communication capabilities. In some embodiments the piezoelectric substrate and/or other elements such as a storage capacitor are off-chip. For example, the piezoelectric substrate and the storage capacitor may be assembled on a board containing the chip.

In some embodiments, the chip-based fluorescence detector further comprises an on-chip antenna configured to receive power from an external transducer and supply power for operation of the chip-based fluorescence detector, wherein electrical energy output from the on-chip antenna in response to receiving the power is stored in the on chip-energy storage device. In some embodiments, the antenna is configured to receive RF power from an external RF transducer and supply power for operation of the chip-based fluorescence detector, wherein electrical energy output from the on-chip antenna in response to receiving the RF power is stored in the on chip-energy storage device. In other embodiments, the antenna is configured to receive electromagnetic power inductively transferred to a coil from an external transducer.

In certain embodiments, an energy storage device such as a capacitor or a rechargeable battery is used to store electrical energy output from a piezoelectric substrate in response to receiving ultrasound power or an antenna in response to receiving RF power or electromagnetic power, wherein the capacitor or rechargeable battery supplies power to the plurality of light emitting sources and the array of fluorescence-sensing pixels. In certain embodiments this energy storage device is on-chip or off-chip. In some embodiments the piezoelectric substrate and/or battery or storage capacitor are assembled on a solid support (e.g., board) containing the chip. In certain embodiments, the chip-based fluorescence detector comprises a precharged battery, and does not receive external power or recharge. After discharging the battery remains until it is removed.

The chip-based fluorescence detector may further comprise several regulators to generate the on-chip voltages for circuit operation and on-chip clock generation. In embodiments where power is transferred from an external transducer, the chip-based fluorescence detector further comprises a voltage rectifier and several voltage regulators to generate the on-chip voltages for circuit operation. Multiple voltage sources at various levels can be generated on-chip. For example, 3.3 V, 2.5 V, 2.1 V, 1.8 V and 1 V can be generated, which can then directly power circuitry on the chip. The imager may further comprise a clock, which can be derived from the transducer signal. An analog to digital converter (ADC) can also be included on the chip. In certain embodiments the ADC is an 8-bit differential SAR ADC with a 0.5 V or 1 V maximum range. In other embodiments, the ADC is a 10-bit SAR ADC with a 0.5 V or 1 V maximum range.

In some embodiments, 4 ADCs run simultaneously to decrease the conversion time, and their digital outputs are stored in an on-chip storage unit. Storage of digital output of the ADC minimizes the effect of leakage as the pixels are being read out sequentially and transmitted according to the communication protocol. This approach won't change the overall power consumption for analog to digital conversion since the total number of pixels is the same.

In some embodiments, the in-pixel sampling switches are made of thicker oxide devices to limit the leakage current due to subthreshold conduction of the devices during hold times. Thicker oxide devices cut down the leakage current by more than 2 orders of magnitude. In other embodiments, the transient decay of the individual pixel's voltage due to leakage can be estimated and characterized by conducting a series of measurements on the same pixel at different timepoints. The reconstructed transient voltage waveform for each pixel is used to estimate the initial value of the pixel voltage by extrapolation.

In some embodiments, in-pixel ADCs minimize the analog to digital conversion time for the pixel array and circumvent the leakage problem and enable expanding the size of the imager array without compromising the frame rate. Multiple ADC types or architectures can be used. One example, to maintain the fill factor of the pixels, is to use an oversampling Sigma-Delta.

In certain embodiments, the ASIC further comprises a reference voltage generator. In some embodiments, the reference voltage generator is a bandgap voltage reference generator. In some embodiments, due to the well-regulated body temperature, the on-chip reference voltage generator is proportional to absolute temperature (PTAT). In certain embodiments, the chip-based fluorescence detector further comprises a power-on-reset (POR) circuit.

In some embodiments, the chip-based fluorescence detector further comprises a data storage unit in communication with the array of fluorescence-sensing pixels, wherein the data storage unit stores imaging data from the array of fluorescence-sensing pixels. The data storage component may be of any type capable of storing information, and may utilize, e.g., FLASH memory, metal-oxide-semiconductor (MOS) memory, random-access memory (RAM), dynamic random-access memory (DRAM), static random-access memory (SRAM), synchronous dynamic random-access memory (SDRAM), or any other write-capable memory.

In some embodiments, the chip-based fluorescence detector further comprises a data processing unit in communication with the data storage unit. Image processing may include local background subtraction, whereby values from neighboring pixels are used to measure the background. Image processing may include averaging multiple sequential images to improve the signal to noise ratio within the image. The images are taken within a time span such that the cell positions do not appreciably change (for example within a few minutes). Images taken with different wavelengths of light in rapid succession can be used to (1) calibrate out non-uniformities in illumination, and (2) auto-fluorescence background and (3) dark current, offsets and low frequency noise processes, such as flicker noise. In some embodiments, machine learning techniques are employed in processing imaging data to improve image quality. Machine learning algorithms may also be used to automate analysis of imaging data and for classification of cells. Any suitable machine learning algorithm may be used in processing imaging data including, without limitation, convolutional neural networks (CNNs) and deep learning algorithms.

In some embodiments, the chip-based fluorescence detector further comprises an edge computing device connected to the data storage unit, wherein the edge computing device receives data from the data storage unit. An on-chip edge computing device may be programmed to partially process fluorescence imaging data, which is subsequently transmitted to an external data processing unit to complete data processing.

In certain embodiments, the chip-based fluorescence detector further comprises a first wireless communication unit in communication with the data storage unit and an external data receiving device comprising a second wireless communication unit. In some embodiments, the first wireless communication unit utilizes a wireless communication protocol using an electromagnetic carrier wave (e.g., radio wave, microwave, or infrared) or ultrasound to transfer data from the data storage unit to the external data receiving device comprising the second wireless communication unit. For example, the first wireless communication unit may utilize a radio-frequency communication protocol or an ultrasound communication protocol to transfer data from the data storage unit to the external data receiving device comprising the second wireless communication unit. The data receiving device may include, without limitation, a computer or handheld device, such as a cell phone or tablet. In certain embodiments, the chip-based fluorescence detector periodically takes images and stores data in the data storage unit on the chip. At either set time points or upon interrogation, the stored data can be wireless transmitted from the chip-based fluorescence detector to the external data receiving device.

In certain embodiments, the chip-based fluorescence detector further comprises an analog to digital converter in communication with the data storage unit. In certain embodiments, the chip-based fluorescence detector further comprises multiple analog to digital converters which then feed data into a storage buffer or memory which is then transmitted. This data can be transmitted at the time of imaging, or stored for subsequent transmission. The data can be averaged with subsequent images taken near the same time point.

In certain embodiments, the chip-based fluorescence detector further comprises a backscattering modulator unit in communication with the array of fluorescence-sensing pixels. In other embodiments, active telemetry modulation schemes such as amplitude shift keying (ASK), phase shift keying (PSK), frequency shift keying (FSK), pulse width modulation amplitude shift keying (PWM-ASK), pulse position modulation (PPM) and spectrally efficient quadrature amplitude modulation (QAM) can be used for data communication. In other embodiments, power and data carrier frequencies are separated to maximize bandwidth for data communication while minimizing the tissue attenuation by lowering the frequency of external carrier waveforms during power transfer. It may also have the ability to wirelessly transmit data. See, e.g., M. J. Karimi, A. Schmid and C. Dehollain, “Wireless Power and Data Transmission for Implanted Devices via Inductive Links: A Systematic Review,” in IEEE Sensors Journal, vol. 21, no. 6, pp. 7145-7161, 15 Mar. 15, 2021; herein incorporated by reference in its entirety).

In certain embodiments, the chip-based fluorescence detector further comprises a digital state machine that controls the stage of operation of the chip. For example, the stages may include i) power up, during which energy is stored in the energy storage device for chip operation, ii) illumination and imaging, which may last for 10 ms to 100 ms (but may be shorter or longer), iii) data conversion and iv) transmission. During the illumination stage, the light-emitting sources (e.g., laser diodes or LEDs) are turned on. In some embodiments, during the illumination stage, a laser diode is used that is supplied by a 33 mA current for 50 ms. In some embodiments, the chip can be programed to supply a set voltage and current to the light source. In some embodiments, multiple light sources are placed on chip, each with a different voltage supply and current supply requirement. Just prior to the illumination stage, pixels are reset, and any offset is measured and stored in pixels. During the illumination stage, the pixel is activated and integrates fluorescent emitted light. At the end of illumination, the pixel value is sampled and held in an in-pixel storage capacitor. Both the offset value and the fluorescence value from each pixel are serially fed to the analog to digital converter. The analog to digital converter converts this analog signal to a digital value which is transmitted off chip. In other embodiments, these values are stored on-chip and transmitted at a later time on demand. In some embodiments multiple wavelengths of light are used, and images are sequentially taken as described, each using a single wavelength light source. In some embodiments, multiple light sources are used (such as light sources on the imager and separate micro-stars). In these cases, the light sources of the same wavelength on different chips can be used simultaneously or sequentially. These stages can be triggered from an external transducer or guided by an internal clock. It is understood to those skilled in the art that a variety of circuit design approaches can be used to accomplish these stages of operation. In order to obtain semi-continuous monitoring (every minute, hour, day, etc.) over long periods of time (hours to days to months), without having the patient tethered to an external device to provide continuous power and data transfer, an on-chip rechargeable battery and on-chip memory storage may be added to the chip-based fluorescence detector.

The frequency of sensing (fluorescence imaging) is determined by the biologic process and the signal-to-noise ratio needed for accuracy. The sampling frequency (i.e., how many sensing operations per second) depends on the speed of the biological process. Preferably, the sampling frequency is at least two times faster than any significant biological change to accurately sample the environment. Sampling may be every second, minute, hour, day, or week depending on the process (or any time interface in between). The signal to noise ratio can be improved by acquiring multiple images that can be averaged together. Each image in this scenario must be taken before a significant change in the environment has occurred (see, e.g., Gharia et al. (2020) Mol Imaging 19:1536012120913693), herein incorporated by reference). To obtain the power necessary to accomplish this without tethering the patient to an external transducer continuously, an on-chip rechargeable battery can be used.

A clock can be added to the chip if the clock is not derived from an external source. The on-chip battery charges an on-chip capacitor to provide the high instantaneous currents needed for optical illumination and chip operation (which exceed those that the battery can provide). Once the capacitor is charged, chip operation proceeds as above, with illumination and image capture, and then analog to digital conversion. In some embodiments the power is supplied by an on-chip rechargeable battery. The digital output is then stored on chip by a data storage unit. For images that are averaged to improve SNR, on chip averaging and then storage of the averaged image can be performed on chip. Images are obtained at predefined intervals, and the process is repeated. This interval (and the number of averages) can be programed on chip or transmitted to the chip. At periodic intervals, an external transceiver interrogates the chip and signals for data upload. Data upload is performed either using US or RF back-scatter or active uplink. The battery is then recharged using ultrasound-based power transfer or RF based power transfer, and the memory is reset. The process repeats again. Other power sources can also be used. This includes energy harvesting, momentum harvesting, and nuclear power harvesting. The above process can also be used on the micro-stars (illumination only) without the imager and requisite data storage and upload.

In certain embodiments, the chip-based fluorescence detector further comprises an envelope detector for the AC input, the output of which controls a finite state machine (FSM). The FSM generates multiple control/timing signals for proper operation of the internal blocks of the imager such as sample and conversion times of the analog to digital converter. The FSM also performs packet scheduling and timing of the data uplink. The output of the envelope detector also acts as a downlink data stream that is used to wirelessly configure the imager settings such as integration time. In other embodiments wherein a precharged battery replaces the AC input, an input control signal keeps track of the transitions of the FSM by overriding the output of the timer control module.

For a further description of chip-based fluorescence detectors, which can be incorporated into the dual modality imager, see also International Patent Application Publication No. WO 2022/165234 A1; herein incorporated by reference in its entirety.

### Imaging Agents

Before imaging tissue of a subject using the dual modality imager, the subject is administered one or more imaging agents that selectively label one or more target cells with fluorophores and gamma-emitting radionuclides. Each imaging agent comprises a fluorophore or gamma-emitting radionuclide or both a fluorophore and a gamma-emitting radionuclide conjugated to a binding agent that selectively binds directly or indirectly to a marker on a target cell of interest. In some embodiments, imaging with the dual modality imager is performed with one or more imaging agents comprising one or more fluorophores having a fluorescence emission in the visible, near-infrared (NIR), or infrared (IR) region of the light spectrum, Preferred imaging wavelengths are within the optical window of tissue, extending from approximately 500 nm to 900 nm.

In certain embodiments, imaging with the dual modality imager is performed with one or more fluorophores having a fluorescence emission in the visible region of the light spectrum, which ranges from about 380 nm to 780 nm. Exemplary fluorophores with emissions in the visible region of the light spectrum include, without limitation, SYBR green, SYBR gold, a CAL Fluor dye such as CAL Fluor Gold 540, CAL Fluor Orange 560, CAL Fluor Red 590, CAL Fluor Red 610, and CAL Fluor Red 635, a Quasar dye such as Quasar 570, Quasar 670, and Quasar 705, an Alexa Fluor such as Alexa Fluor 488, Alexa Fluor 546, Alexa Fluor 555, Alexa Fluor 594, Alexa Fluor 647, and Alexa Fluor 784, a cyanine dye such as Cy3, Cy3.5, Cy5, Cy5.5, and Cy7, fluorescein, 2′, 4′, 5′, 7′-tetrachloro-4-7-dichlorofluorescein (TET), carboxyfluorescein (FAM), fluorescein isothiocyanate (FITC), 6-carboxy-4′,5′-dichloro-2′,7′-dimethoxyfluorescein (JOE), hexachlorofluorescein (HEX), rhodamine, carboxy-X-rhodamine (ROX), tetramethyl rhodamine (TAMRA), and Texas Red.

In certain embodiments, imaging with the dual modality imager is performed with one or more fluorophores having a fluorescence emission in the NIR region of the light spectrum, which ranges from about 700 nm to 1700 nm. The use of NIR fluorophores is advantageous in minimizing interference from tissue autofluorescence and enhancing tissue penetration compared to other fluorophores. Preferably, the fluorescent emission wavelength is in a region of the spectrum where blood and tissue absorb minimally, and tissue penetration is maximal, such as in the range from 700 nm to 1000 nm. An imaging agent may comprise any NIR fluorophore with an emission in the NIR region of the spectrum, including, but not limited to, fluorophores with fluorescence emissions at about 700, 720, 740, 760, 780, 800, 820, 840, 860, 880, 900, 920, 940, 960, 980, or 1000 nm, or any wavelength in between. Exemplary NIR fluorophores include, without limitation, IRDye dyes (e.g., IRDye 800CW, IRDye 680RD, IRDye 700, IRDye 750, and IRDye 800RS), CF dyes (e.g., CF680, CF680R, CF750, CF770, and CF790), Tracy dyes (e.g., Tracy 645 and Tracy 652), Alexa dyes (e.g., Alexa Fluor® 660 dye, Alexa Fluor® 700 dye, Alexa Fluor® 750 dye, and Alexa Fluor® 790), cyanine dyes (e.g., Cy7 and Cy7.5), thienothiadiazole dyes, phthalocyanine dyes, squaraine dyes, rhodamine dyes and analogues (e.g., Si-pyronine, Si-rhodamine, Te-rhodamine, and Changsha), borondipyrromethane (BODIPY) dyes, seminaphthofluorone xanthene dyes, benzo[c] heterocycle dyes (e.g., isobenzofuran dyes), and quantum dots. For a review of NIR fluorophores and their use in fluorescence imaging, see, e.g., Escobedo et al. (2010) Curr. Opin.

Chem. Biol. 14 (1): 64-70, Hilderbrand et al. (2010) Curr. Opin. Chem. Biol. 14 (1): 71-79, Yuan et al. (2013) Chem. Soc. Rev. 42 (2): 622-661, Vats et al. (2017) Int. J. Mol. Sci. 18 (5), Zhang et al. (2017) Nat. Rev. Clin. Oncol. 14 (6): 347-364, Gao et al. (2010) Curr. Top. Med. Chem. 10 (12): 1147-1157, Zhao et al. (2018) Wiley Interdiscip Rev Nanomed Nanobiotechnol. 10 (3): e1483, and Haque et al. (2017) Bioorg. Med. Chem. 25 (7): 2017-2034; herein incorporated by reference in their entireties.

Imaging with the dual modality imager can be performed with one or more imaging agents comprising one or more gamma-emitting radionuclides. Exemplary gamma-emitting radionuclides include, without limitation, ⁴⁶Sc, ⁶⁴Cu, ⁶⁷Ga, ^{99m}Tc, ¹¹¹In, ¹²³I, ¹³¹I, ¹³³Ba, ¹⁵⁵Tb, ¹⁷⁷Lu, ¹³³Xe, and ²⁰¹TI.

In some embodiments, both a fluorophore and gamma-emitting radionuclide are present in an imaging agent. In other embodiments, the fluorophore and gamma-emitting radionuclide are in separate imaging agents and the separate imaging agents are co-administered to the subject. When in separate imaging agents, the radionuclide and the fluorophore may be conjugated to the same binding agent or different binding agents. Examples of binding agents include, without limitation, antibodies, antibody fragments, antibody mimetics, and aptamers as well as small molecules, peptides, peptoids, or ligands that bind selectively to target cellular markers. The binding agent may selectively bind to any type of molecule, including proteins, lipids, polysaccharides, proteoglycans, metabolites, or the like. In some embodiments, multiple imaging agents comprising radionuclides and fluorophores having fluorescent emissions at different wavelengths are used to label cellular markers on target cells, wherein the different imaging agents bind to different markers on target cells of the same cell-type or different cell-types.

In some embodiments, the binding agent selectively binds to a cellular marker on a lesion such as, but not limited to, a tumor, diseased tissue, a congenital malformation, or damaged or abnormal tissue. In some embodiments, the binding agent binds to a lesion marker of interest with high affinity. One or more imaging agents comprising fluorophores and gamma-emitting radionuclides may be used, for example, for detecting a single cell type or multiple cell types within a lesion. The dual modality imager can simultaneously image both fluorescence and gamma radiation emitted by imaging agents bound to the lesion. In some embodiments, an imaging agent comprises a binding agent that selectively binds to a cancer cell-specific marker. Fluorophores and radionuclides may be conjugated to any binding agent that specifically binds to a cancer marker of interest such as a tumor-specific antigen or tumor-associated antigen.

In certain embodiments, the binding agent comprises an antibody that specifically binds to a cellular marker of interest. Any type of antibody may be used in conjugates with gamma-emitting radionuclides and/or fluorophores, including, without limitation, monoclonal antibodies, polyclonal antibodies, as well as hybrid antibodies, altered antibodies, chimeric antibodies, and humanized antibodies. Antibodies may include hybrid (chimeric) antibody molecules (see, for example, Winter et al. (1991) Nature 349:293-299; and U.S. Pat. No. 4,816,567); F(ab′) 2 and F(ab) fragments; Fv molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (scFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B: 120-126); diabodies, tetrabodies, affibodies, camelid antibodies, humanized antibody molecules (see, e.g., Riechmann et al. (1988) Nature 332:323-327; Verhoeyan et al. (1988) Science 239:1534-1536; and U.K. Patent Publication No. GB 2,276, 169, published 21 Sep. 1994); and, any functional fragments obtained from such molecules, wherein such fragments retain specific-binding properties of the parent antibody molecule.

In other embodiments, the binding agent comprises an aptamer that specifically binds to a cellular marker of interest. Any type of aptamer may be used, including a DNA, RNA, xeno-nucleic acid (XNA), or peptide aptamer that specifically binds to the tumor antigen. Such aptamers can be identified, for example, by screening a combinatorial library. Nucleic acid aptamers (e.g., DNA or RNA aptamers) that bind selectively to a target tumor antigen can be produced by carrying out repeated rounds of in vitro selection or systematic evolution of ligands by exponential enrichment (SELEX). Peptide aptamers that bind to a marker of interest may be isolated from a combinatorial library and improved by directed mutation or repeated rounds of mutagenesis and selection. For a description of methods of producing aptamers, see, e.g., Aptamers: Tools for Nanotherapy and Molecular Imaging (R.N. Veedu ed., Pan Stanford, 2016), Nucleic Acid and Peptide Aptamers: Methods and Protocols (Methods in Molecular Biology, G. Mayer ed., Humana Press, 2009), Nucleic Acid Aptamers: Selection, Characterization, and Application (Methods in Molecular Biology, G. Mayer ed., Humana Press, 2016), Aptamers Selected by Cell-SELEX for Theranostics (W. Tan, X. Fang eds., Springer, 2015), Cox et al. (2001) Bioorg. Med. Chem. 9 (10): 2525-2531; Cox et al. (2002) Nucleic Acids Res. 30 (20): e108, Kenan et al. (1999) Methods Mol. Biol. 118:217-231; Platella et al. (2016) Biochim. Biophys. Acta November 16 pii: S0304-4165 (16) 30447-0, and Lyu et al. (2016) Theranostics 6 (9): 1440-1452; herein incorporated by reference in their entireties.

In other embodiments, the binding agent comprises an antibody mimetic. Any type of antibody mimetic may be used, including, but not limited to, affibody molecules (Nygren (2008) FEBS J. 275 (11): 2668-2676), affilins (Ebersbach et al. (2007) J. Mol. Biol. 372 (1): 172-185), affimers (Johnson et al. (2012) Anal. Chem. 84 (15): 6553-6560), affitins (Krehenbrink et al. (2008) J. Mol.

Biol. 383 (5): 1058-1068), alphabodies (Desmet et al. (2014) Nature Communications 5:5237), anticalins (Skerra (2008) FEBS J. 275 (11): 2677-2683), avimers (Silverman et al. (2005) Nat. Biotechnol. 23 (12): 1556-1561), darpins (Stumpp et al. (2008) Drug Discov. Today 13 (15-16): 695-701), fynomers (Grabulovski et al. (2007) J. Biol. Chem. 282 (5): 3196-3204), and monobodies (Koide et al. (2007) Methods Mol. Biol. 352:95-109).

In other embodiments, the binding agent comprises a small molecule ligand. Small molecule ligands encompass numerous chemical classes, e.g., small organic compounds having a molecular weight of less than about 10,000 daltons, less than about 5,000 daltons, or less than about 2,500 daltons. The small molecule will include one or more functional groups necessary for structural interaction with the target analyte, e.g., groups necessary for hydrophobic, hydrophilic, electrostatic or even covalent interactions. Where the target analyte is a protein (e.g., cellular marker), the ligand will include functional groups necessary for structural interaction with proteins, such as hydrogen bonding, hydrophobic-hydrophobic interactions, electrostatic interactions, etc., and will typically include at least an amine, amide, sulfhydryl, carbonyl, hydroxyl or carboxyl group, or preferably at least two of the functional chemical groups. The small molecule may also comprise a region that may be modified and/or participate in conjugation to a radionuclide, without substantially adversely affecting the small molecule's ability to bind to its target analyte.

Small molecule ligands can comprise cyclical carbon or heterocyclic structures and/or aromatic or polyaromatic structures substituted with one or more of the above functional groups. Small molecule ligands may also include organic compounds comprising alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Small molecule ligands are also found among biomolecules including peptides, carbohydrates, fatty acids, steroids, purines, pyrimidines, derivatives, structural analogs, or combinations thereof. The small molecule may be derived from a naturally occurring or synthetic compound that may be obtained from a wide variety of sources, including libraries of synthetic or natural compounds. For example, numerous means are available for random and directed synthesis of a wide variety of organic compounds and biomolecules, including the preparation of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known small molecules may be subjected to directed or random chemical modifications, such as acylation, alkylation, esterification, amidification, etc. to produce structural analogs.

As such, the small molecule may be obtained from a library of naturally occurring or synthetic molecules, including a library of compounds produced through combinatorial means, i.e., a compound diversity combinatorial library. When obtained from such libraries, the small molecule employed will have demonstrated some desirable affinity for the protein target in a convenient binding affinity assay. Combinatorial libraries, as well as methods for the production and screening, are known in the art and described in: U.S. Pat. Nos. 5,741,713; 5,734,018; 5,731,423; 5,721,099; 5,708,153; 5,698,673; 5,688,997; 5,688,696; 5,684,711; 5,641,862; 5,639,603; 5,593,853; 5,574,656; 5,571,698; 5,565,324; 5,549,974; 5,545,568; 5,541,061; 5,525,735; 5,463,564; 5,440,016; 5,438,119; 5,223,409, the disclosures of which are herein incorporated by reference.

Small molecule ligands may also include known drugs that selectively bind to receptors on cells, including, without limitation, growth factor receptors, receptor tyrosine kinases, receptor protein serine/threonine kinases, G-protein coupled receptors, cytokine receptors, lectin receptors, folate receptors, prostate-specific membrane antigen (PSMA), carbonic anhydrase IX receptor, and biotin receptors. For example, anti-cancer drugs that bind to such cellular receptors may be used as ligands to target radionuclides to cancer cells. Exemplary drugs that may be used as ligands to target cancer cells include, without limitation, Acitinib, Afatinib, Axitinib, Erlotinib, Cabozantinib, Crizotinib, Gefitinib, Imatinib, Ibrutinib, Lapatinib, Neovastat, Nilotinib, Pazopanib, Perifosine, Ponatinib, Regorafenib, Sorafenib, Sunitinib, Trametinib, and Vandetenib.

Exemplary tumor-specific antigens and tumor-associated antigens include, without limitation, oncogene protein products, mutated or dysregulated tumor suppressor proteins, oncovirus proteins, oncofetal antigens, mutated or dysregulated differentiation antigens, overexpressed or aberrantly expressed cellular proteins (e.g., mutated or aberrantly expressed growth factors, mitogens, receptor tyrosine kinases, cytoplasmic tyrosine kinases, serine/threonine kinases and their regulatory subunits, G proteins, and transcription factors), and altered cell surface glycolipids and glycoproteins on cancerous cells. For example, tumor-specific antigens and tumor-associated antigens may include without limitation, dysregulated or mutated RAS, WNT, MYC, ERK, TRK, CTAG1B, MAGEA1, Bcr-Abl, p53, c-Sis, epidermal growth factor receptor (EGFR), platelet-derived growth factor receptor (PDGFR), vascular endothelial growth factor receptor (VEGFR), HER2/neu, Src-family, Syk-ZAP-70 family proteins, and BTK family of tyrosine kinases, Abl, Raf kinase, cyclin-dependent kinases, alphafetoprotein (AFP), carcinoembryonic antigen (CEA), CA-125, MUC-1, epithelial tumor antigen (ETA), tyrosinase, melanoma-associated antigen (MAGE), and other abnormal or dysregulated proteins expressed on cancerous cells. In some embodiments, the cancer-targeted binding agent binds to a tumor antigen of interest with high affinity.

In certain embodiments, the tumor marker targeted by a binding agent is the urokinase plasminogen activator receptor (uPAR) or urokinase plasminogen activator (uPA). A number of anti-uPAR antibodies are available including the 2G10 antibody, which inhibits the uPAR interaction with urokinase plasminogen activator, and anti-uPAR antibody, 3C6, which inhibits the association of uPAR with β1 integrin (see, e.g., LeBeau et al. (2013) Cancer Res. 73 (7): 2070-2081). Anti-PAR and anti-uPA antibodies can be conjugated to radionuclides for use in imaging or radioligand therapy for treatment of cancerous cells expressing uPAR or uPA, respectively, including, without limitation, those of breast cancer including triple negative breast cancer, pancreas cancer, prostate cancer, and melanoma.

In certain embodiments, the tumor marker targeted by a binding agent is PD-L1. A number of anti-PD-L1 antibodies are commercially available including durvalumab, pembrolizumab, atezolizumab and avelumab. Other anti-PD-L1 antibodies include C4 and DFO-C4 (see, e.g., Truillet C et al. (2018) Bioconjug. Chem. 29 (1): 96-103). Such anti-PD-L1 antibodies can be conjugated to radionuclides for use in imaging or radioligand therapy for treatment of cancerous cells expressing PD-L1, including, without limitation, those of melanoma, lung cancer, including non-small cell lung cancer (NSCLC) and small cell lung cancer (SCLC), head and neck cancer, Hodgkin lymphoma, stomach cancer, prostate cancer, bladder cancer, urothelial carcinoma, breast cancer including triple-negative breast cancer (TNBC), hepatocellular carcinoma (HCC), Merkel cell carcinoma, and renal cell carcinoma.

In certain embodiments, the tumor marker targeted by a binding agent is the epidermal growth factor receptor (EGFR). A number of anti-EGFR antibodies are available including panitumumab, cetuximab, zalutumumab, nimotuzumab, and matuzumab, which can be conjugated to radionuclides for use in imaging or radioligand therapy for treatment of cancerous cells expressing EGFR, including, without limitation, those of head and neck cancer, colorectal cancer, lung cancer, ovarian cancer, breast cancer, endometrial cancer, cervical cancer, bladder cancer, gastric cancer, and esophageal cancer. A number of small molecule drugs are also available that target EGFR including, without limitation, Gefitinib, Erlotinib, Lapatinib, Sorafenib, and Vandetenib, which can be conjugated to radionuclides for use in imaging or radioligand therapy for treatment of cancerous cells expressing EGFR, according to the methods described herein.

In other embodiments, the tumor marker targeted by a binding agent is HER2. A number of anti-HER2 antibodies are also available including trastuzumab, pertuzumab, and margetuximab, which can be conjugated to radionuclides for use in imaging or radioligand therapy for treatment of cancerous cells expressing HER2, including, without limitation, those of breast cancer, ovarian cancer, stomach cancer, lung cancer, uterine cancer, gastric cancer, colon cancer, head and neck cancer, and salivary duct carcinoma. A number of small molecule drugs are also available that target HER2 including, without limitation, Lapatinib and Neratinib, which can be conjugated to radionuclides for use in imaging or radioligand therapy for treatment of cancerous cells expressing HER2, according to the methods described herein.

In other embodiments, the tumor marker targeted by a binding agent is the epithelial cell adhesion molecule (EpCAM) 17-1A. A number of anti-EpCAM 17-1A antibodies are also available including edrecolomab, catumaxomab, and nofetumomab, which can be conjugated radionuclides for use in imaging or radioligand therapy for treatment of cancerous cells expressing EpCAM 17-1A to detect cancerous cells in epithelial-derived neoplasms and various carcinomas, such as lung cancer, gastrointestinal cancer, breast cancer, ovarian cancer, pancreatic cancer, renal cancer, cervical cancer, colorectal cancer, and bladder cancer.

In other embodiments, the tumor marker targeted by a binding agent is CD20. A number of anti-CD20 antibodies are also available including rituximab, tositumomab, ocrelizumab, obinutuzumab, ocaratuzumab, ofatumumab, ibritumomab tiuxetan, ublituximab, and veltuzumab, which can be conjugated to radionuclides for use in imaging or radioligand therapy for treatment of cancerous cells expressing CD20, including, without limitation, those of lymphoma such as, but not limited to, marginal zone lymphoma, Hodgkins lymphoma, non-Hodgkins lymphoma; leukemia such as, but not limited to, chronic lymphocytic leukemia, acute lymphoblastic leukemia, myelogenous leukemia, and chemotherapy-resistant hairy cell leukemia; and thyroid cancer.

In other embodiments, the tumor marker targeted by a binding agent is CD52. A number of anti-CD52 antibodies are also available including alemtuzumab, which can be conjugated radionuclides for use in imaging or radioligand therapy for treatment of cancerous cells expressing CD52, including, without limitation, those of lymphoma such as, but not limited to, cutaneous T-cell lymphoma (CTCL) and T-cell lymphoma and chronic lymphocytic leukemia (CLL).

In other embodiments, the tumor marker targeted by a binding agent is CD22. A number of anti-CD22 antibodies are also available including inotuzumab, which can be conjugated to radionuclides for use in imaging or radioligand therapy for treatment of cancerous cells expressing CD22, including, without limitation, those of leukemia such as, but not limited to, lymphoblastic leukemia and hairy cell leukemia; lymphoma, and lung cancer.

In other embodiments, the tumor antigen targeted by a binding agent is CD19. A number of anti-C19 antibodies are also available including blinatumomab, MEDI-551 and MOR-208, which can be conjugated to radionuclides for use in imaging or radioligand therapy for treatment of cancerous cells expressing CD19, including, without limitation, those of B-cell neoplasms, non-Hodgkin lymphoma (NHL), chronic lymphocytic leukemia (CLL), acute lymphoblastic leukemia (ALL), and multiple myeloma (MM).

In certain embodiments, the tumor marker targeted by a binding agent is carcinoembryonic antigen (CEA). A number of anti-CEA antibodies are available including arcitumomab, which can be conjugated to radionuclides for use in imaging or radioligand therapy for treatment of cancerous cells expressing CEA, including, without limitation, those of colorectal carcinoma, gastric carcinoma, pancreatic carcinoma, lung carcinoma, breast carcinoma, and medullary thyroid carcinoma.

In certain embodiments, the tumor marker targeted by a binding agent is prostate-specific membrane antigen (PSMA). A number of anti-PSMA antibodies are available including capromab, PSMA30 nanobody, and IAB2M minibody, which can be conjugated to radionuclides for use in imaging or radioligand therapy for treatment of cancerous cells expressing PSMA, including, without limitation, those of prostate cancer. A number of small molecule drugs are also available that target PSMA including, without limitation, zinc binding compounds linked to a glutamate isostere or glutamate, phosphonate-, phosphate-, and phosphoramidates and ureas, fluciclovine (Axumin), MIP-1072, MIP-1095, N-(N-((S)-1,3-dicarboxypropyl) carbamoyl)-4-(18F) fluorobenzyl-L-cysteine (18F-DCFBC), which can be conjugated to radionuclides for use in imaging or radioligand therapy for treatment of cancerous cells expressing PSMA, according to the methods described herein.

In certain embodiments, the tumor marker targeted by a binding agent is the folate receptor (FR). A number of anti-FR antibodies are available including farletuzumab and m909, which can be conjugated to radionuclides for use in imaging or radioligand therapy for treatment of cancerous cells expressing FR, including, without limitation, those of ovarian cancer, breast cancer, lung cancer, pleura cancer, cervical cancer, endometrial cancer, kidney cancer, bladder cancer and brain cancer, The small molecule, folate, can also be conjugated to radionuclides for use in imaging or radioligand therapy for treatment of cancerous cells expressing FR, according to the methods described herein.

In certain embodiments, the tumor marker targeted by a binding agent is a matrix-metalloproteinase (MMP), including, without limitation, MMP1, MMP3, MMP7, MMP9, MMP10, MMP11, MMP12, MMP13, and MMP14. A number of anti-MMP antibodies are available including, which can be conjugated to radionuclides for use in imaging or radioligand therapy for treatment of cancerous cells expressing MMPs, including, without limitation, those of ovarian cancer, breast cancer, lung cancer, prostate cancer, stomach cancer, thyroid cancer, skin cancer, brain cancer, kidney cancer, colon cancer, bladder cancer, esophageal cancer, endometrial cancer, hepatocellular cancer, and head and neck cancer. Endogenous glycoprotein inhibitors such as tissue inhibitor of metalloproteinases (TIMPs), including TIMP-1, TIMP-2, TIMP-3, and TIMP-4 as well as a number of small molecule drugs are available that target MMPs including, without limitation, doxycycline, marimastat (BB-2516), and cipemastat, which can be conjugated to fluorophores and radionuclides for use in imaging or radioligand therapy for treatment of cancerous cells expressing MMPs, according to the methods described herein.

In some embodiments, an imaging agent comprises a binding agent that selectively binds to a lymphatic marker to allow imaging of the lymphatics system by the dual modality imager. In some embodiments, the marker targeted by a binding agent is a lymphatic vessel marker such as, but not limited to, lymphatic vessel endothelial hyaluronan receptor 1 (LYVE-1), podoplanin (PDPN), Prospero homeobox 1 (PROX1), Fms-like tyrosine kinase receptor 4 (FLT-4), CD31, CD34, and D2-40.

In some embodiments, an imaging agent comprises a binding agent that selectively binds to a vein marker to allow imaging of veins by the dual modality imager. In some embodiments, the marker targeted by a binding agent is a vein marker such as, but not limited to, receptor subfamily 2 group F member 2 (NR2F2), EPH receptor B4 (EPHB4), and SMARCA4 SWI/SNF related BAF chromatin remodeling complex subunit ATPase 4 (SMARCA4).

In some embodiments, an imaging agent comprises a binding agent that selectively binds to an artery marker to allow imaging of arteries by the dual modality imager. In some embodiments, the marker targeted by a binding agent is an artery marker such as, but not limited to, kinase insert domain receptor (KDR), notch receptor 1 (NOTCH1), notch receptor 3 (NOTCH3), notch receptor 4 (NOTCH4), delta like canonical Notch ligand 4 (DELTA4), jagged canonical Notch ligand 1 (JAG1), jagged canonical Notch ligand 2 (JAG2), and ephrin-B2 (EFNB2).

Chelating agents can be included in imaging agents to confer metal binding capability, wherein the chelating agent forms a complex with a gamma-emitting radionuclide metal ion. Exemplary chelating agents include, without limitation, 1,4,7,10-tetraazacyclododecane-1,4,7,10-tetraacetic acid (DOTA), 1,4,7-triazacyclononane-N,N′, N″-triacetic acid (NOTA), ethylenediaminetetraacetic acid (EDTA), diethylenetriaminepentaacetic acid (DTPA), 1,4,7-triazacyclononane-N,N′, N″-triacetic acid (NOTA), ({4-[2-(bis-carboxymethyl-amino)-ethyl]-7-carboxymethyl-[1,4, 7]triazonan-1-yl}acetic acid (NETA), and p-bromoacetamido-benzyl-acid (TETA), porphyrins, polyamines, crown ethers, bis-tetraethylaminetetraacetic thiosemicarbazones, polyoximes, and like groups known to be useful for this purpose.

Fluorophores may be conjugated to binding agents by any suitable method. In some instances, the fluorophore and binding agent are directly linked, e.g., via a single bond, or indirectly linked, e.g., through the use of a suitable linker, e.g., a polymer linker, a chemical linker, or one or more linking molecules or moieties. In some instances, attachment of the fluorophore and binding agent may be by way of one or more covalent interactions. In some instances, the fluorophore or binding agent may be functionalized, e.g., by addition or creation of a reactive functional group. Functionalized fluorophores or binding agents may be modified to contain any convenient reactive functional group for conjugation such as an amine functional group, a carboxylic functional group, a sulfhydryl group, a thiol functional group, and the like.

Any convenient method of bioconjugation may be used including, but not limited to, glutaraldehyde crosslinking, carbodiimide crosslinking, succinimide ester crosslinking, imidoester, crosslinking, maleimide crosslinking, iodoacetamide crosslinking, benzidine crosslinking, periodate crosslinking, isothiocyanate crosslinking, and the like. Such conjugation methods may optionally use a reactive sidechain group of an amino acid residue of the binding agent (e.g., a reactive side-chain group of a Lys, Cys, Ser, Thr, Tyr, His or Arg amino acid residue of the protein, i.e., a polypeptide linking group may be amino-reactive, thiol-reactive, hydroxyl-reactive, imidazolyl-reactive or guanidinyl-reactive). In some cases, a chemoselective reactive functional group may be utilized. Other conjugation reagents that can be used include, but are not limited to, e.g., homobifunctional conjugation reagents (e.g., (bis(2-[succinimidooxycarbonyloxy]ethyl) sulfone, 1,4-Di-(3′-[2′pyridyldithio]-propionamido) butane, disuccinimidyl suberate, disuccinimidyl tartrate, sulfodisuccinimidyl tartrate, dithiobis(succinimidyl propionate), 3,3′-dithiobis(sulfosuccinimidyl propionate), ethylene glycol bis(succinimidyl succinate), and the like), heterobifunctional conjugation reagents (e.g., m-maleimidobenzoyl-N-hydroxysuccinimide ester, m-maleimidobenzoyl-N-hydroxysulfosuccinimide ester, N-γ-maleimidobutyryloxysuccinimide ester, N-γ-maleimidobutyryloxysulfosuccinimide ester, N-(8-maleimidocaproic acid) hydrazide, N-(ε-maleimidocaproyloxy) succinimide ester, N-(8-maleimidocaproyloxy) sulfo succinimide ester, N-(p-maleimidophenyl) isocyanate, N-succinimidyl(4-iodoacetyl)aminobenzoate, succinimidyl 4-(N-maleimidomethyl)cyclohexane-1-carboxylate, succinimidyl 4-(p-maleimidophenyl) butyrate, N-sulfosuccinimidyl(4-iodoacetyl)aminobenzoate, sulfosuccinimidyl 4-(N-maleimidomethyl)cyclohexane-1-carboxylate, sulfo succinimidyl 4-(p-maleimidophenyl) butyrate, I-ethyl-3-(3-dimethylaminopropyl) carbodiimide hydrochloride, I-ethyl-3-(3-dimethylaminopropyl) carbodiimide hydrochloride, maleimide PEG N-hydroxysuccinimide ester, and the like), photoreactive conjugation reagents (e.g., p-azidobenzoyl hydrazide, N-5-azido-2-nitrobenzyloxysuccinimide, p-azidophenyl glyoxal monohydrate, N-(4-[p-azidosalicylamido] butyl)-3′-(2′-pyridyldithio) propionamide, bis(P-[4-azidosalicylamido]-ethyl)disulfide, N-hydroxysuccinimideyl-4-azidosalicyclic acid, N-hydroxysulfosuccinimidyl-4-azidobenzoate, sulfosuccinimidyl 2-(7-azido-4-methylcoumarin-3-acetamide)ethyl-1,3-dithiopropionate, azido phenyl 2-(m-azido-o-nitrobenzamido)-ethyl-1,3′-propionate, sulfosuccinimidyl 6-(4′-azido-2′-nitrophenylamino) hexanoate, sulfosuccinimidyl(4-azidophenyl dithio) propionate, sulfosuccinimidyl-2-(p-azidosalicylamido)ethyl-1,3-dithiopropionate, and the like).

In some instances, attachment of a fluorophore to a binding agent of interest is mediated by one or more functional linkers. A functional linker, as used herein, refers to any suitable linker that has one or more functional groups for the attachment of one molecule to another. For example, in some instances the functional linker comprises an amino functional group, a thiol functional group, a hydroxyl functional group, an imidazolyl functional group, a guanidinyl functional group, an alkyne functional group, an azide functional group, or a strained alkyne functional group. Further exemplary functional groups and methods of crosslinking and conjugation are described in, e.g., Hermanson Bioconjugate Techniques (Academic Press, 3^{rd} edition, 2013), herein incorporated by reference in its entirety.

### Systems and Computer Implemented Methods

The present disclosure provides systems and computer implemented methods which find use in practicing the subject methods. In some embodiments, the system includes a dual modality imager as described herein, an external or internal power source; and an external data receiving device.

In certain embodiments, the external power source is an ultrasound transducer, an electromagnetic (EM) transducer, an inductive transducer, or a radiofrequency (RF) transducer. The system may also comprise an internal power source such as a battery, a radionuclide, a photovoltaic system, or a radionuclide in combination with a scintillator and photovoltaic energy harvester. The external power source may be used, for example, to charge an on-chip battery that stores and provides power to a chip comprising a fluorescence detector or gamma photon detector.

In a further aspect, the external data receiving device may include an external processor, a storage component (i.e., memory), a display component, and other components typically present in general purpose computers. In some embodiments, the external data receiving device is a computer or handheld device (e.g., a cell phone or tablet). The storage component stores information accessible by the processor, including instructions that may be executed by the processor and data that may be retrieved, manipulated, or stored by the processor. The processor is programmed to process fluorescence and gamma radiation imaging data received from the dual modality imager and display an image showing the fluorescence detected by the fluorescence-sensing pixels and the gamma photons detected by the gamma photon-sensing pixels of the dual modality imager. The processor can be programmed to perform steps of a computer implemented method comprising: receiving imaging data from the array of gamma photon-sensing pixels and the array of fluorescence-sensing pixels of the dual modality imager after administering a detectably effective amount of an imaging agent to a subject, wherein the imaging agent comprises a fluorophore and a gamma-emitting radionuclide conjugated to a binding agent that selectively binds to a cellular marker; and displaying an image showing the fluorescence detected by the fluorescence-sensing pixels and the gamma photons detected by the gamma photon-sensing pixels. In certain embodiments, the computer-implemented method further comprises determining the size and depth of a lesion in tissue of the subject based on the relative energies of the incoming gamma photons measured by the gamma photon-sensing pixels. In certain embodiments, the computer-implemented method further comprises displaying on the image the size and the depth of the lesion in the tissue. In certain embodiments, the method further comprises determining the size and depth of a lesion in tissue of the subject based on the relative energies of the incoming gamma photons measured by the gamma photon-sensing pixels. In some embodiments, the computer-implemented method further comprises displaying on the image the size and the depth of the lesion in the tissue.

In certain embodiments, the computer-implemented method further comprises determining relative energies and angles of incidence of the incoming gamma photons that hit the array of gamma photon-sensing pixels. In certain embodiments, the computer-implemented method further comprises identifying the gamma-emitting radionuclide source of each incoming gamma photon that hits the array of gamma photon-sensing pixels. In certain embodiments, the computer-implemented method further comprises quantifying energy deposition on a diode (E_{dep}) by an incoming gamma photon and voltage signal decay time (DT) after the incoming gamma photon hits the diode.

The methods can be implemented in digital electronic circuitry, or in computer software, firmware, or hardware. The disclosed and other embodiments can be implemented as one or more computer program products, i.e., one or more modules of computer program instructions encoded on a computer readable medium for execution by, or to control the operation of, a data processing apparatus. The computer readable medium can be a machine-readable storage device, a machine-readable storage substrate, a memory device, a composition of matter effecting a machine-readable propagated signal, or any combination thereof.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

In certain embodiments, the external data receiving device further comprises a wireless communication unit in communication with a wireless communication unit of the dual modality imager. The wireless communication unit may utilize a wireless communication protocol, for example, using an electromagnetic carrier wave (e.g., a radio wave, microwave, or an infrared carrier wave) or ultrasound to receive data from the internal data storage unit of the dual modality imager.

The storage component includes instructions. For example, the storage component includes instructions for processing imaging data from the array of gamma photon-sensing pixels and the array of fluorescence-sensing pixels of the dual modality imager. The computer processor is coupled to the storage component and configured to execute the instructions stored in the storage component in order to receive fluorescence and gamma photon imaging data from the dual modality imager and process the data using one or more algorithms, as described herein. The display component displays the processed imaging data.

The processor and/or memory may be operably connected to a display device, for example, via a wired, such as a Universal Serial Bus (USB) connection, or wireless connection, such as a Bluetooth connection. Any convenient display device, such as a liquid crystal display (LCD), light-emitting diode (LED) display, plasma (PDP) display, quantum dot (QLED) display or cathode ray tube display device may be used. The display component displays information regarding whether the renal transplant recipient is over-immunosuppressed or under-immunosuppressed.

The storage component may be of any type capable of storing information accessible by the processor, such as a hard-drive, memory card, ROM, RAM, DVD, CD-ROM, USB Flash drive, write-capable, and read-only memories. The processor may be a general purpose processor, a graphics processor unit, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor can also include primarily analog components. A computing environment can include any type of computer system, including, but not limited to, a computer system based on a microprocessor, a graphics processor unit, a mainframe computer, a digital signal processor, a portable computing device, a personal organizer, a device controller, and a computational engine within an appliance, to name a few.

The steps of a method, process, or algorithm described in connection with the embodiments disclosed herein can be embodied directly in hardware, in a software module executed by a processor, or in a combination of the two. A software module, engine, and associated databases can reside in memory resources such as in RAM memory, FRAM memory, flash memory, ROM memory, EPROM memory, EEPROM memory, registers, hard disk, a removable disk, a CD-ROM, or any other form of non-transitory computer-readable storage medium, media, or physical computer storage known in the art. An exemplary storage medium can be coupled to the processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data.

In certain embodiments, the processor and storage component may comprise multiple processors and storage components that may or may not be stored within the same physical housing. For example, some of the instructions and data may be stored on removable CD-ROM and others within a read-only computer chip. Some or all of the instructions and data may be stored in a location physically remote from, yet still accessible by, the processor. Similarly, the processor may comprise a collection of processors which may or may not operate in parallel.

In some embodiments, the method can be performed using a cloud computing system. In these embodiments, the imaging data from the array of gamma photon-sensing pixels and the array of fluorescence-sensing pixels of the dual modality imager and the programming can be exported to a cloud computer, which runs the program, and returns an output to the user.

The system may also include at least one imaging agent (i.e., a fluorophore and/or gamma-emitting radionuclide conjugated to a binding agent that selectively binds to a cellular marker of interest) for carrying out the methods described herein. In some embodiments, the system includes multiple imaging agents, which can be used in fluorescence and radionuclide gamma photon imaging of multiple markers and cell-types. In certain embodiments, the system includes one or more imaging agents for fluorescence and radionuclide gamma photon imaging of a lesion (e.g., a tumor, diseased tissue, a congenital malformation, or damaged or abnormal tissue), lymphatic vessels, veins, arteries, or a combination thereof. The system may also include one or more external light sources for illumination of tissue with light at an excitation wavelength of an imaging agent fluorophore included in the system.

Components of systems for carrying out the presently disclosed methods are further described in the examples below.

### Combined Fluorescence and Radionuclide Gamma Photon Intraoperative Imaging

Tissue to be imaged is contacted with at least one imaging agent, such that the imaging agent specifically binds to a cellular marker of a cell type of interest. In some embodiments, a detectably effective amount of an imaging agent is administered to a subject; that is, an amount that is sufficient to yield an acceptable image of the fluorescence detected by the fluorescence-sensing pixels and the gamma photons detected by the gamma photon-sensing pixels of the dual modality imager. A detectably effective amount of the imaging agent may be administered in more than one injection if needed. The detectably effective amount of the imaging agent needed for an individual may vary according to factors such as the degree of susceptibility to uptake into the target area of interest, the age, sex, and weight of the individual, and the particular imaging agent that is used. Optimization of such factors is within the level of skill in the art.

The tissue to be imaged is illuminated with light at a fluorescence excitation wavelength of the fluorophore of an imaging agent. In some embodiments, the dual modality imager is used to simultaneously detect fluorescence at multiple wavelengths from multiple imaging agents containing different fluorophores that emit fluorescence at different wavelengths. In such embodiments, the tissue to be imaged is illuminated with light at the fluorescence excitation wavelengths of each of the fluorophores of the imaging agents either simultaneously or sequentially.

Any suitable light source may be used for fluorescence excitation. Exemplary light sources include, without limitation, halogen lamps, light-emitting diodes (LEDs), and laser diodes. The light source may be located on the dual modality imager (e.g., on the chip-based fluorescent detector or elsewhere) or externally. In addition, band-pass filters can be used to produce multiple excitation channels from a light source and narrow the band of excitation. In some embodiments, a light emitting source is located on a chip. Light emitting sources that can be used on a chip include, without limitation, micro-laser diodes and LEDs. In some embodiments, LEDs are used with emission filters. Alternatively, LEDs having a tight enough full-width half maximum spread can be used without emission filters. The light sources may emit light of a single color (wavelength) or multiple colors (wavelengths) so as to illuminate multiple fluorophores (e.g., of multiple imaging agents that bind to different cellular markers). Multiple light sources of the same and/or different wavelength emissions may be arrayed on a chip, and used in parallel or serially to illuminate tissue. In some embodiments, an external light emitting source is on a separate chip from the chip-based fluorescence detector. In some embodiments, a chip comprising an external light emitting source is implanted in tissue. The internal and/or external light sources can be activated together or sequentially.

In some embodiments, the imaging agent is administered to a subject before performing a surgery, wherein the dual modality imager is used to provide combined fluorescence and gamma photon imaging of tissue for surgical guidance. For example, the dual modality imager can be used for combined fluorescence and radionuclide gamma photon imaging of tissue containing a lesion (e.g., a tumor, diseased tissue, a congenital malformation, or damaged or abnormal tissue), lymphatic vessels, veins, or arteries, or any combination thereof. During surgical resection of a tumor, the images of tissue obtained with the dual modality imager can be used to avoid overlooking small areas of tumor buried under tissue, metastases, and microscopic disease, and to assess surgical margins. In some embodiments, a surgical specimen is contacted with a detectably effective amount of an imaging agent after removal of a tumor from a subject and subsequently imaged by the dual modality imager to check surgical margins. Failure to obtain clear surgical margins is associated with recurrence and poor overall survival rates. Therefore, further surgery may be indicated if cancerous cells are detected within a surgical margin in images produced by the dual modality imager.

In certain embodiments, a gamma photon counter is attached to the skin of the subject, wherein the gamma photon counter is used in combination with the gamma photon-sensing pixels of the dual modality imager to determine the location of the radionuclide source of the incoming gamma photons that hit a gamma photon-sensing pixel. In some embodiments, multiple gamma photon counters are attached to the skin of the subject, wherein detection of gamma photons at the known locations of the counters and the gamma photon-sensing pixels of the dual modality imager is used to reconstruct the distribution of gamma emissions within the body. In some embodiments, distributed point source (DPS) modelling is used to generate a distribution of gamma photon-emitting point sources within the boundaries of a lesion, wherein the DPS modeling is used to i) calculate probabilities for each gamma photon counter and each gamma photon-sensing pixel of the dual modality imager that the gamma photons, counted by the gamma photon counter or gamma photon-sensing pixel of the dual modality imager, were received from a gamma photon emitting point source within the boundaries of a particular lesion based on an assumption that counts per second (CPS) falloff correlates with 1/(distance between the centroid location of the gamma photon counter or gamma photon-sensing pixel of the dual modality imager and the gamma photon emitting point source) 2 and CPS values are attenuated by an empirically derived factor Q which accounts for attenuation and scattering of gamma photons in tissue (see, e.g., International Patent Application Publication No. WO 2025/059557; herein incorporated by reference in its entirety).

In certain embodiments, a method of performing intraoperative image-guided surgery on a subject is provided, the method comprising: administering a detectably effective amount of an imaging agent to the subject, wherein the imaging agent comprises a fluorophore and a gamma-emitting radionuclide conjugated to a binding agent that selectively binds to a cellular marker on a lesion (e.g., a tumor, diseased tissue, a congenital malformation, or damaged or abnormal tissue); illuminating tissue with light at a fluorescence excitation wavelength of the fluorophore; performing image-guided surgical resection of the lesion using a dual modality imager, described herein, wherein the dual modality imager produces fluorescence and gamma-emitting radionuclide images of the lesion; moving the dual modality imager around the tissue surrounding the resection site to produce fluorescence and radionuclide images of the tissue surrounding the resection site after performing the image-guided surgical resection of the lesion; using the fluorescence and radionuclide images of the tissue surrounding the resection site to determine if the lesion was completely removed by the surgical resection and to detect additional lesions, if present, wherein the dual modality imager provides information regarding tissue depths and sizes of the additional lesions, if present; and surgically resecting any remaining portion of the lesion or additional lesions that are detected by the dual modality imager. In some embodiments, the method further comprises illuminating the surgical specimen removed from the subject with light at a fluorescence excitation wavelength of the fluorophore; moving the dual modality imager over surgical margins surrounding the lesion in the surgical specimen, wherein fluorescence and gamma-emitting radionuclide images produced by the dual modality imager are used to detect cells of the lesion, if present, in the surgical margins to determine if the surgical specimen comprises a positive margin; and further checking the tissue surrounding the resection site in the subject for the cells of the lesion if the surgical specimen is determined to comprise a positive margin.

In certain embodiments, a method of performing intraoperative lymphatic imaging on a subject is provided, the method comprising: administering a detectably effective amount of an imaging agent to the subject, wherein the imaging agent comprises a fluorophore and a gamma-emitting radionuclide conjugated to a binding agent that selectively binds to a lymphatic marker; illuminating lymphatic tissue with light at a fluorescence excitation wavelength of the fluorophore; and performing image-guided lymphatic surgery using a dual modality imager, described herein, wherein the dual modality imager produces fluorescence and gamma-emitting radionuclide images of the lymphatic system. In certain embodiments, the subject is administered one or more imaging agents comprising binding agents that bind to lymphatic markers on a lymphatic vessel marker. Exemplary lymphatic vessel markers include, without limitation, lymphatic vessel endothelial hyaluronan receptor 1 (LYVE-1), podoplanin (PDPN), Prospero homeobox 1 (PROX1), Fms-like tyrosine kinase receptor 4 (FLT-4), CD31, CD34, or D2-40, or any combination thereof. In certain embodiments, the dual modality imager produces an intraoperative map of lymphatic vessels for real-time surgical guidance. The subject methods can be applied to various types of lymphatic surgeries including, but not limited to, lymphovenous bypass, nodal transplant, lymphovenous anastomosis, and surgical resection of lymphatic tumors. In certain embodiments, the method further comprises moving the dual modality imager over the surgical site to produce fluorescence and radionuclide images of the surgical site after performing the lymphatic surgery; assessing results of the lymphatic surgery using the fluorescence and radionuclide images of the surgical site; and performing further surgically repair of the lymphatic tissue, if needed, based on said assessing the results of the lymphatic surgery.

In certain embodiments, the method further comprises: administering a second imaging agent to the subject, wherein the second imaging agent comprises a second fluorophore and a second gamma-emitting radionuclide conjugated to a second binding agent that selectively binds to a vein marker, wherein the second fluorophore emits fluorescent light at a different wavelength than the fluorophore conjugated to the binding agent that selectively binds to the lymphatic marker; illuminating lymphatic tissue with light at a fluorescence excitation wavelength of the second fluorophore; and performing the image-guided lymphatic surgery using the dual modality imager, wherein the dual modality imager produces fluorescence and radionuclide images of veins and lymphatic system vessels. In certain embodiments, the dual modality imager produces an intraoperative map of veins and lymphatic vessels for real-time surgical guidance.

In certain embodiments, a method of performing intraoperative imaging of an artery or a vein in a subject is provided, the method comprising: administering a detectably effective amount of an imaging agent to the subject, wherein the imaging agent comprises a fluorophore and a gamma-emitting radionuclide conjugated to a binding agent that selectively binds to an artery marker or a vein marker; illuminating the artery or vein with light at a fluorescence excitation wavelength of the fluorophore; and performing image-guided surgery on the artery or vein using the dual modality imager described herein, wherein the dual modality imager produces fluorescence and gamma-emitting radionuclide images of the artery or vein. In certain embodiments, the method further comprises moving the dual modality imager over a surgical site to produce fluorescence and gamma-emitting radionuclide images of the surgical site after performing the surgery on the artery or vein; assessing results of the surgery on the artery or vein using the fluorescence and gamma-emitting radionuclide images of the surgical site; and performing further surgically repair of the artery or vein, if needed, based on assessing the results of surgery on the artery or vein.

In certain embodiments, the subject is administered one or more imaging agents comprising binding agents that bind to markers on an artery or vein. Exemplary artery markers include, without limitation, kinase insert domain receptor (KDR), notch receptor 1 (NOTCH1), notch receptor 3 (NOTCH3), notch receptor 4 (NOTCH4), delta like canonical Notch ligand 4 (DELTA4), jagged canonical Notch ligand 1 (JAG1), jagged canonical Notch ligand 2 (JAG2), and ephrin-B2 (EFNB2). Exemplary vein markers include, without limitation, receptor subfamily 2 group F member 2 (NR2F2), EPH receptor B4 (EPHB4), and SMARCA4 SWI/SNF related BAF chromatin remodeling complex subunit ATPase 4 (SMARCA4).

### Kits

Also provided are kits comprising a dual modality imager, as described herein. In some embodiments, the kit comprises software for carrying out the computer implemented methods, described herein, for integrating fluorescence and gamma-emitting radionuclide imaging data. In some embodiments, the kit comprises a non-transitory computer-readable medium and instructions for displaying an image showing the fluorescence detected by the fluorescence-sensing pixels and the gamma photons detected by the gamma photon-sensing pixels of the dual modality imager. In some embodiments, the kit comprises a system comprising a processor programmed to display an image showing the fluorescence detected by the fluorescence-sensing pixels and the gamma photons detected by the gamma photon-sensing pixels of the dual modality imager.

The kit may further include at least one imaging agent (i.e., comprising a fluorophore and/or gamma-emitting radionuclide conjugated to a binding agent that selectively binds to a cellular marker of interest) for carrying out the methods described herein. In some embodiments, the kit includes multiple imaging agents, which can be used in fluorescence and radionuclide gamma photon imaging of multiple markers. In certain embodiments, the kit includes one or more imaging agents for fluorescence and radionuclide gamma photon imaging of a lesion (e.g., a tumor, diseased tissue, a congenital malformation, or damaged or abnormal tissue), lymphatic vessels, veins, or arteries, or a combination thereof.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-85 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. a Dual Modality Imager Comprising:
   a first chip comprising an array of gamma photon-sensing pixels, wherein each gamma photon-sensing pixel provides in-pixel signal amplification for incoming gamma photons that hit the gamma photon-sensing pixel and in-pixel data processing to determine relative energies and angles of incidence of the incoming gamma photons, and wherein each gamma photon-sensing pixel only outputs data from the in-pixel data processing in response to an incoming gamma photon hitting the gamma photon-sensing pixel; and
   a second chip comprising an array of fluorescence-sensing pixels, wherein the second chip is positioned relative to the first chip such that the incoming gamma photons pass through the second chip to reach the array of gamma photon-sensing pixels.
2. The dual modality imager of aspect 1, wherein the dual modality imager can be used to perform simultaneous imaging of gamma photons and fluorescence.
3. The dual modality imager of aspect 1 or 2, wherein the dual modality imager can be used to perform intraoperative image-guided surgery.
4. The dual modality imager of aspect 3, wherein the in-pixel data processing further determines tissue depths of gamma-emitting radionuclide sources from which the incoming gamma photons originate.
5. The dual modality imager of any one of aspects 1-4, wherein the imaging of gamma photons is single photon emission computed tomography (SPECT) imaging.
6. The dual modality imager of any one of aspects 1-5, wherein the fluorescence-sensing pixels detect fluorescent light in a near-infrared or visible region of the electromagnetic spectrum.
7. The dual modality imager of any one of aspects 1-6, wherein the dual modality imager further comprises a handle extending away and disposed angularly from the first chip.
8 The dual modality imager of any one of aspects 1-7, wherein each gamma photon-sensing pixel comprises a silicon diode, wherein a gamma photon incident on a surface of the silicon diode breaks a silicon bond to produce an electron-hole pair, wherein a pulse of charge (Qₚ) is generated that is accumulated by a diode capacitor resulting in generation of a voltage pulse.
9. The dual modality imager of any one of aspects 1-8, wherein the array of gamma photon-sensing pixels comprises a plurality of energy-resolving pixels, a plurality of low-flux pixels, and a plurality of energy-calibrating pixels, wherein each energy-resolving pixel comprises a reverse-biased diode that generates a voltage drop at a diode node in response to energy deposition by an incoming gamma photon hitting the reverse-biased diode, wherein the reverse-biased diode is connected to a unity-gain buffer, wherein output of the unity-gain buffer is connected to an operational transconductance amplifier (OTA) with a direct current (DC) compensation loop that secures an output DC level of the OTA, wherein output of the OTA is routed to a p-channel metal-oxide-semiconductor (PMOS) common-source (CS) stage with a configurable load to set a custom sensitivity of the energy-resolving pixel, wherein the load is configured with a 3-bit in-pixel six transistor (6T) static random-access memory (SRAM) that is used to control in-pixel digital-to-analog converters (DACs) that set a gate voltage of the load; and two inverters, wherein voltage pulses are digitized using the two inverters and sent to a digital processing unit via a set of switches, wherein widths of the digital pulses are measured to estimate a voltage signal decay time (DT);
   wherein each low-flux pixel comprises 6 reverse-biased diodes, wherein each reverse-biased diode is coupled to a unity-gain buffer, wherein buffer outputs from each unity-gain buffer are connected together and routed to an OTA comprising a unity-gain buffer in a feedback loop, said OTA serving as a pseudoresistor to create a summing amplifier with gain=−1, wherein summation of individual gamma photon hits on any of the 6 reverse-biased diodes is routed to an open-loop OTA comprising a DC compensation loop, N-type metal-oxide-semiconductor (NMOS)CS-stage with an SRAM-configurable load, and inverters for count digitization; and wherein the energy-calibrating pixels comprise a plurality of reverse-biased diodes of varying diode sizes having different diode parasitic capacitances and different voltage drops in response to energy deposition by an incoming gamma photon, wherein the plurality of reverse-biased diodes are connected to a unity-gain buffer, wherein output of the unity-gain buffer is connected to an OTA with a DC compensation loop that secures an output DC level of the OTA, wherein output of the OTA is routed to a PMOS CS stage with a configurable load to set a custom sensitivity of the energy-calibrating pixel, wherein the load is configured with a 3-bit in-pixel 6T SRAM that is used to control in-pixel DACs that set a gate voltage of the load; and two inverters, wherein voltage pulses are digitized using the two inverters and sent to a digital processing unit via a set of switches, wherein the energy-calibrating pixels are used for energy-binning to determine the maximum energy deposited (E_{dep}) by a gamma photon, wherein a least significant bit (LSB) is selected to optimize energy resolution and dynamic range, and wherein the LSB is used to estimate the DT.
10. The dual modality imager of aspect 9, wherein the varying diode sizes of the energy-calibrating pixels comprise diode areas of 4.41 μm², 4.84 μm², 5.29 μm², 5.76 μm², and 6.25 μm².
11. The dual modality imager of aspect 9 or 10, wherein the array of gamma photon-sensing pixels comprises a plurality of rows of energy-resolving pixels, a plurality of rows of low-flux pixels, and a plurality of rows of energy-calibrating pixels, wherein each row in the array of gamma photon-sensing pixels shares a count-line (CL) and a count column line (CCL), wherein the CL outputs a pulse whenever a gamma photon is detected by a gamma photon-sensing pixel, wherein numbers of pulses output by the CL are measured with a 10-bit counter, wherein non-overlapping column address sweep (CAS) signals are routed down every column of the array of gamma photon-sensing pixels such that when a gamma photon is detected, the CCL outputs a pulse to identify the column address, wherein the DT, column, and row data are priority encoded, queued into a first in first out (FIFO) memory element, serialized, and sent to a field programmable gate array (FPGA).
12. The dual modality imager of any one of aspects 9-11, further comprising an SRAM sensitivity calibration unit, wherein the SRAM sensitivity calibration unit is used to read or write to the 3-bit in-pixel SRAM to control gain of each CS-stage.
13. The dual modality imager of aspect 12, wherein each gamma photon-sensing pixel is initially calibrated in absence of gamma radiation, wherein SRAM values are increased to increase the CS gain until the CL is triggered by noise, wherein a maximum CS gain threshold is selected at below the level at which the CL is triggered by noise.
14. The dual modality imager of any one of aspects 1-8, wherein each gamma photon-sensing pixel comprises:
   a silicon diode;
   a unity gain voltage amplifier connected to the silicon diode, wherein each voltage pulse generated by a gamma photon is individually buffered by the unity gain voltage amplifier;
   a differential closed-loop amplifier, wherein buffered voltage outputs from the unity gain voltage amplifier are connected to inputs of the differential closed-loop amplifier, wherein voltage gain is either pre-set or configurable using in-pixel memory and a digital-to-analog converter (DAC), wherein the voltage pulse generated across the silicon diode is buffered and amplified by a fixed process-invariant gain;
   an inverter chain comprising a plurality of inverters connected to amplified voltage output from the differential closed-loop amplifier, wherein the inverter chain generates digitized output corresponding to each voltage pulse generated by a gamma photon;
   a digital counter coupled to the digitized output of the inverter chain, wherein the digital counter counts gamma photon detection events, wherein each gamma photon detection event corresponds to the voltage pulse produced by each gamma photon incident on the surface of the silicon diode;
   an on-chip data buffer configured to store a plurality of gamma photon count records for a plurality of gamma photon detection events;
   a digital clock configured to produce a clock signal on the chip, wherein the digital counter uses the clock signal to count numbers of gamma photon detection events per a period of time;
   an energy storage device located on the first chip or externally; and a custom digital logic circuit configured to control voltages and supply power from the energy storage device to the array of gamma photon-sensing pixels, the digital counter, and the digital clock.
15. The dual modality imager of any one of aspects 1-8, wherein each gamma photon-sensing pixel comprises a pair of silicon diodes comprising a first silicon diode and a second silicon diode, wherein a gamma photon incident on a surface of either the first silicon diode or the second silicon diode breaks a silicon bond to produce an electron-hole pair, wherein a pulse of charge (Qₚ) is generated that is accumulated by a diode capacitor resulting in generation of a voltage pulse.
16. The dual modality imager of aspect 15, wherein the first chip further comprises: a unity gain voltage amplifier connected to each silicon diode, wherein each voltage pulse generated by a gamma photon is individually buffered by the unity gain voltage amplifier;
   a differential amplifier, wherein buffered voltage outputs from the unity gain voltage amplifier are connected to inputs of the differential amplifier;
   a voltage integrator, wherein the voltage integrator accepts a selected DC voltage and sets voltage output of the differential amplifier, wherein the voltage integrator is bootstrapped from the differential amplifier output to the differential amplifier input in a negative feedback configuration;
   a pair of level shifters connected to output of the differential amplifier, wherein the pair of level shifters comprises two level shifters comprising a first level shifter that shifts a direct current (DC) level of the output of the differential amplifier up to amplify only the voltage pulse from the first diode, and a second level shifter that shifts the DC level of the differential amplifier output down to only amplify the voltage pulse from the second diode, wherein an amount the DC level is shifted is set using on-chip configurable memory and an in-pixel digital to analog converter (DAC) to convert stored bits into an analog shift in voltage;
   an inverter chain comprising a plurality of inverters connected to amplified and shifted voltage output from the pair of level shifters, wherein the inverter chain generates digitized output corresponding to each voltage pulse generated by a gamma photon;
   a digital counter coupled to the digitized output of the inverter chain, wherein the digital counter counts gamma photon detection events, wherein each gamma photon detection event corresponds to the voltage pulse produced by each gamma photon incident on the surface of either the first silicon diode or the second silicon diode;
   an on-chip data buffer configured to store a plurality of gamma photon count records for a plurality of gamma photon detection events;
   a digital clock configured to produce a clock signal on the chip, wherein the digital counter uses the clock signal to count numbers of gamma photon detection events per a period of time;
   an energy storage device located on the first chip or externally; and
   a custom digital logic circuit configured to control voltages and supply power from the energy storage device to the array of gamma photon-sensing pixels, the digital counter, and the digital clock.
17. The dual modality imager of any one of aspects 8-16, wherein the silicon diodes are reversed biased or have zero voltage bias.
18. The dual modality imager of any one of aspects 1-17, wherein the first chip is covered with a Compton scattering material.
19. The dual modality imager of aspect 18, wherein the Compton scattering material comprises lead, tungsten, or bismuth.
20. The dual modality imager of aspect 18 or 19, wherein the Compton scattering material is in a layer on top of the first chip or on the back of the first chip.
21. The dual modality imager of any one of aspects 1-20, wherein the first chip further comprises on-chip circuitry tuned to be responsive to a range of linear energy transfer (LET) from incoming gamma photons such that combination and distribution of signals from gamma photon-sensing pixels with known LET responsivity enables determination of incident energies of the incoming gamma photons.
22. The dual modality imager of aspect 21, wherein the array of gamma photon-sensing pixels comprises a first subset of gamma photon-sensing pixels and a second subset of gamma photon-sensing pixels with known LET responsivity, wherein the first subset of gamma photon-sensing pixels has a lower gain than the second subset of gamma photon-sensing pixels, wherein the first subset of gamma photon-sensing pixels detects lower energy photons having higher LET but not higher energy gamma photons having lower LET, wherein the second set of gamma photon-sensing pixels detects both the lower energy gamma photons having higher LET and the higher energy gamma photons having lower LET.
23. The dual modality imager of any one of aspects 1-22, wherein the gamma-emitting radionuclide is ⁴⁶Sc, ⁶⁴Cu, ⁶⁷Ga, ^{99m}Tc, ¹¹¹In, ¹²³I, ¹³¹I, ¹³³Ba, ¹⁵⁵Tb, ¹⁷⁷Lu, ¹³³Xe, or ²⁰¹TI.
24. The dual modality imager of any one of aspects 1-23, wherein each fluorescence-sensing pixel comprises a photodiode, wherein florescent light incident on a surface of the photodiode is converted into an electrical current proportional to intensity of the incident florescent light.
25. The dual modality imager of aspect 24, wherein the photodiode is a silicon photodiode, a germanium photodiode, or an indium gallium arsenide photodiode.
26 The dual modality imager of any one of aspects 1-25, wherein the array of fluorescence-sensing pixels comprises a plurality of photodiodes arrayed on the surface of the second chip, wherein each photodiode is coated with at least one layer of filter material that functions as an optical filter.
27. The dual modality imager of any one of aspects 1-26, wherein the second chip further comprises:
   a plurality of light emitting sources to provide excitation light, wherein the plurality of light emitting sources are located on the second chip or externally;
   an energy storage device to supply power for operation of the second chip, wherein the energy storage device is located on the second chip or externally;
   a data storage unit in communication with the array of fluorescence-sensing pixels, wherein the data storage unit stores imaging data from the array of fluorescence-sensing pixels; and
   an application-specific integrated circuit (ASIC) configured to control voltages and supply power from the energy storage device to the array of fluorescence-sensing pixels, the plurality of light emitting sources on the second chip, and the data storage unit.
28. The dual modality imager of aspect 27, wherein the light emitting sources are micro-laser diodes or light-emitting diodes.
29. The dual modality imager of claim 28, wherein the plurality of light-emitting diodes further comprises an emission filter on each light-emitting diode.
30. The dual modality imager of any one of aspects 27-29, wherein the energy storage device to supply power for operation of the second chip comprises a battery, a capacitor, a radionuclide, a photovoltaic system, or a radionuclide in combination with a scintillator and photovoltaic energy harvester.
31. The dual modality imager of any one of aspects 27-30, wherein the plurality of light emitting sources located externally are implantable in tissue.
32. The dual modality imager of aspect 31, wherein the plurality of light emitting sources located externally are micro-star light sources.
33. The dual modality imager of any one of aspects 27-32, wherein an external power source supplies power to the plurality of light emitting sources located on the second chip or externally.
34. The dual modality imager of any one of aspects 26-33, wherein said at least one layer of filter material comprises or consists of amorphous silicon, crystalline silicon, gallium phosphide, cadmium selenide, gallium arsenide, or indium phosphide.
35. The dual modality imager of any one of aspects 26-33, wherein the thickness of the layer of filter material is varied on the plurality of photodiodes to allow selection of light at different fluorescence emission wavelengths for multiple fluorophores having different fluorescence emission spectra.
36. The dual modality imager of any one of aspects 26-35, wherein a band gap and the thickness of the layer of filter material is chosen to allow selection of near-infrared light or visible light at a fluorescence emission wavelength of a fluorophore of interest.
37. The dual modality imager of any one of aspects 26-36, wherein the optical filter is an absorption filter or an interference filter.
38. The dual modality imager of aspect 37, wherein the absorption filter has a band gap and thickness suitable to allow light at a fluorescence emission wavelength of a fluorophore of interest to pass through to the photodiode.
39. The dual modality imager of aspect 37, wherein the interference filter is a single bandpass, dual bandpass, triple bandpass, or quadruple bandpass interference filter.
40. The dual modality imager of any one of aspects 37-39, wherein the interference filter further comprises a layer of absorption filter material on top of the interference filter or underneath the interference filter.
41. The dual modality imager of any one of aspects 37-40, wherein the interference filter further comprises one or more layers of material comprising a plurality of angle selective gratings, collimators, or fiber optic plates that blocks light that is not incident within 30° of an axis perpendicular to the plane of the second chip.
42. The dual modality imager of any one of aspects 37-41, wherein the interference filter further comprises one or more layers of material comprising a plurality of fiber optic plates that blocks light that is not incident within 6° of an axis perpendicular to the plane of the second chip.
43. The dual modality imager of any one of aspects 37-42, wherein the interference filter further comprises one or more layers of material comprising a plurality of angle selective gratings that blocks light that is not incident within 15° of an axis perpendicular to the plane of the second chip.
44. The dual modality imager of aspect 43, wherein said one or more layers of material comprising a plurality of angle selective gratings, collimators, or fiber optics are on top of the layer of filter material, underneath the layer of filter material, or both on top and underneath the layer of filter material.
45. The dual modality imager of aspect 44, wherein said layer on top of the layer of filter material blocks light that is not incident within 10°-15° of an axis perpendicular to the plane of the chip.
46. The dual modality imager of aspect 44 or 45, wherein said layer underneath the layer of filter material blocks light that is not incident within 5°-30° of an axis perpendicular to the plane of the chip.
47. A computer-implemented method for integrating fluorescence and gamma-emitting radionuclide imaging data, the computer performing steps comprising:
   receiving imaging data from the array of gamma photon-sensing pixels and the array of fluorescence-sensing pixels of the dual modality imager of any one of aspects 1-46 after administering a detectably effective amount of an imaging agent to a subject, wherein the imaging agent comprises a fluorophore and a gamma-emitting radionuclide conjugated to a binding agent that selectively binds to a cellular marker; and
   displaying an image showing the fluorescence detected by the fluorescence-sensing pixels and the gamma photons detected by the gamma photon-sensing pixels.
48. The computer-implemented method of aspect 47, further comprising:
   determining size and depth of a lesion in tissue of the subject based on the relative energies of the incoming gamma photons measured by the gamma photon-sensing pixels; and
   displaying on the image the size and the depth of the lesion in the tissue.
49. The computer-implemented method of aspect 47 or 48, further comprising determining relative energies and angles of incidence of the incoming gamma photons that hit the array of gamma photon-sensing pixels.
50. The computer-implemented method of any one of aspects 47-49, further comprising identifying the gamma-emitting radionuclide source of each incoming gamma photon that hits the array of gamma photon-sensing pixels.
51. The computer-implemented method of any one of aspects 47-50, further comprising quantifying energy deposition on a diode (E_{dep}) by an incoming gamma photon and voltage signal decay time (DT) after the incoming gamma photon hits the diode.
52. A non-transitory computer-readable medium comprising program instructions that, when executed by a processor in a computer, causes the processor to perform the method of any one of aspects 47-51.
53. A kit comprising the non-transitory computer-readable medium of aspect 52 and instructions for using the dual modality imager for fluorescence and gamma-emitting radionuclide imaging.
54. A system comprising:
   the dual modality imager of any one of aspects 1-46;
   a processor;
   an external data receiving device connected to the processor, wherein the external data receiving device receives imaging data from the array of gamma photon-sensing pixels and the array of fluorescence-sensing pixels of the dual modality imager; and
   a display component that can display an image showing the fluorescence detected by the fluorescence-sensing pixels and the gamma photons detected by the gamma photon-sensing pixels.
55. The system of aspect 54, further comprising an imaging agent comprising a fluorophore and a gamma-emitting radionuclide conjugated to a binding agent that selectively binds to a cellular marker.
56. The system of aspect 55, wherein the cellular marker is a tumor marker.
57. The system of aspect 55, wherein the cellular marker is a disease-associated marker.
58. The system of any one of aspects 55-57, wherein the binding agent is an antibody, an antibody mimetic, an aptamer, or a ligand.
59. The system of any one of aspects 55-58, wherein the imaging agent further comprises a nanoparticle, wherein the binding agent, fluorophore, and a gamma-emitting radionuclide are attached to the nanoparticle.
60. The system of any one of aspects 55-59, further comprising a plurality of imaging agents, wherein each imaging agent comprises a different binding agent that selectively binds to a different cellular marker, and wherein each imaging agent comprises a different fluorophore.
61. The system of any one of aspects 54-59, further comprising a first imaging agent comprising a fluorophore conjugated to a first binding agent that selectively binds to a first cellular marker and a second imaging agent comprising a gamma-emitting radionuclide conjugated to a second binding agent that selectively binds to a second cellular marker.
62. A method of performing intraoperative image-guided surgery on a subject, the method comprising:
   administering a detectably effective amount of an imaging agent to the subject, wherein the imaging agent comprises a fluorophore and a gamma-emitting radionuclide conjugated to a binding agent that selectively binds to a cellular marker on a lesion;
   illuminating tissue with light at a fluorescence excitation wavelength of the fluorophore;
   performing image-guided surgical resection of the lesion using the dual modality imager of any one of aspects 1-46, wherein the dual modality imager produces fluorescence and radionuclide images of the lesion;
   moving the dual modality imager around the tissue surrounding the resection site to produce fluorescence and radionuclide images of the tissue surrounding the resection site after said performing the image-guided surgical resection of the lesion;
   using the fluorescence and radionuclide images of the tissue surrounding the resection site to determine if the lesion was completely removed by the surgical resection and to detect additional lesions, if present, wherein the dual modality imager provides information regarding tissue depths and sizes of the additional lesions, if present; and
   surgically resecting any remaining portion of the lesion or additional lesions that are detected by the dual modality imager.
63. The method of aspect 62, further comprising illuminating the surgical specimen removed from the subject with light at a fluorescence excitation wavelength of the fluorophore;
   moving the dual modality imager over surgical margins surrounding the lesion in the surgical specimen, wherein fluorescence and radionuclide images produced by the dual modality imager are used to detect cells of the lesion, if present, in the surgical margins to determine if the surgical specimen comprises a positive margin; and
   further checking the tissue surrounding the resection site in the subject for the cells of the lesion if the surgical specimen is determined to comprise a positive margin.
64. The method of aspect 62 or 64, wherein the lesion is a tumor, diseased tissue, a congenital malformation, or damaged or abnormal tissue.
65. The method of any one of aspects 62-64, wherein the binding agent is an antibody, an antibody mimetic, an aptamer, or a ligand.
66. The method of any one of aspects 62-65, wherein the radionuclide images are single photon emission computed tomography (SPECT) images.
67. The method of any one of aspects 62-66, wherein the fluorophore emits fluorescent light in a near-infrared or visible region of the electromagnetic spectrum.
68. The method of any one of aspects 62-67, further comprising administering a plurality of imaging agents to the subject, wherein each imaging agent comprises a different binding agent that selectively binds to a different cellular marker, and wherein each imaging agent comprises a different fluorophore.
69. The method of any one of aspects 62-68, further comprising obtaining a pre-operative image of the subject after the subject is administered an imaging agent comprising a gamma-emitting radionuclide conjugated to a binding agent that selectively binds to the cellular marker on the lesion, wherein the pre-operative image shows locations of the gamma-emitting radionuclide in tissue of the subject.
70. The method of any one of aspects 62-69, further comprising attaching a gamma photon counter to the skin of the subject, wherein the gamma photon counter is used in combination with the dual modality imager to determine the location of the radionuclide source of the incoming gamma photons that hit a gamma photon-sensing pixel.
71. A method of performing intraoperative lymphatic imaging on a subject, the method comprising:
   administering a detectably effective amount of an imaging agent to the subject, wherein the imaging agent comprises a fluorophore and a gamma-emitting radionuclide conjugated to a binding agent that selectively binds to a lymphatic marker;
   illuminating lymphatic tissue with light at a fluorescence excitation wavelength of the fluorophore; and
   performing image-guided lymphatic surgery using the dual modality imager of any one of aspects 1-46, wherein the dual modality imager produces fluorescence and radionuclide images of the lymphatic system.
72. The method of aspect 71, wherein the dual modality imager produces an intraoperative map of lymphatic vessels for real-time surgical guidance.
73. The method of aspect 71 or 72, wherein the lymphatic surgery is a lymphovenous bypass, a nodal transplant, a lymphovenous anastomosis, or a surgical resection of a lymphatic tumor.
74. The method of any one of aspects 71-73, wherein the lymphatic marker is a lymphatic vessel marker.
75. The method of aspect 74, wherein the lymphatic vessel marker is lymphatic vessel endothelial hyaluronan receptor 1 (LYVE-1), podoplanin (PDPN), Prospero homeobox 1 (PROX1), Fms-like tyrosine kinase receptor 4 (FLT-4), CD31, CD34, or D2-40, or any combination thereof.
76. The method of any one of aspects 71-75, wherein the binding agent is an antibody, an antibody mimetic, an aptamer, or a ligand.
77. The method of any one of aspects 71-76, wherein the radionuclide images are single photon emission computed tomography (SPECT) images.
78. The method of any one of aspects 71-77, wherein the fluorophore emits fluorescent light in a near-infrared or visible region of the electromagnetic spectrum.
79. The method of any one of aspects 71-78, further comprising administering a plurality of imaging agents to the subject, wherein each imaging agent comprises a different binding agent that selectively binds to a different cellular marker, and wherein each imaging agent comprises a different fluorophore
80. The method of any one of aspects 71-79, further comprising obtaining a pre-operative image of the subject after the subject is administered an imaging agent comprising a gamma-emitting radionuclide conjugated to a binding agent that selectively binds to the lymphatic marker, wherein the pre-operative image shows locations of the gamma-emitting radionuclide in the lymphatic system of the subject.
81. The method of any one of aspects 71-80, further comprising attaching a gamma photon counter to the skin of the subject, wherein the gamma photon counter is used in combination with the dual modality imager to determine the location of the radionuclide source of the incoming gamma photons that hit a gamma photon-sensing pixel.
82. The method of any one of aspects 71-81, further comprising:
   moving the dual modality imager over a surgical site to produce fluorescence and radionuclide images of the surgical site after said performing the lymphatic surgery;
   assessing results of the lymphatic surgery using the fluorescence and radionuclide images of the surgical site; and
   performing further surgically repair of the lymphatic tissue, if needed, based on said assessing the results of the lymphatic surgery.
83. The method of any one of aspects 71-82, further comprising:
   administering a second imaging agent to the subject, wherein the second imaging agent comprises a second fluorophore and a second gamma-emitting radionuclide conjugated to a second binding agent that selectively binds to a vein marker, wherein the second fluorophore emits fluorescent light at a different wavelength than the fluorophore conjugated to the binding agent that selectively binds to the lymphatic marker;
   illuminating lymphatic tissue with light at a fluorescence excitation wavelength of the second fluorophore; and
   performing the image-guided lymphatic surgery using the dual modality imager, wherein the dual modality imager produces fluorescence and radionuclide images of veins and lymphatic system vessels.
84. The method of aspect 83, wherein the dual modality imager produces an intraoperative map of veins and lymphatic vessels for real-time surgical guidance.
85. The method of aspect 74, wherein the vein marker is receptor subfamily 2 group F member 2 (NR2F2), EPH receptor B4 (EPHB4), SMARCA4 SWI/SNF related BAF chromatin remodeling complex subunit ATPase 4 (SMARCA4), or any combination thereof.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

### Example 1

### A Dual Modality Intraoperative Imager

### Overview

Approximately 60% of cancer patients undergo surgery to remove a primary tumor, and precise removal of all cancer cells is vital for optimal outcomes [1] [2]. Microscopic clusters of cancer cells are intraoperatively hard to visualize and are often left behind, significantly increasing the risk of cancer recurrence. In addition to optical intraoperative imaging, radioguided surgery (RGS) is one technique that has been shown to help prevent this. This method involves conjugating a gamma photon (v)-emitting radioisotope to a cancer-targeting molecule and administering this radioactive tracer intravenously to the patient before surgery where it preferentially binds to cancer cells. However, RGS suffers from spatial resolution and is not optimal for margin imaging, but it is better geared towards clusters of cells below the tissue surface. Therefore, a fusion of surface imaging with optical imaging and RGS for depth imaging address both areas-imaging tumor at the surface and those below the surface.

During surgery, a γ-sensing probe is used to locate areas with γ signal to remove all cancerous lesions, and since γ's have superior tissue penetration properties compared to visible photons used in microscopy, deeper lesions can be sensed as well. State-of-the-art γ probes show the benefit of RGS but lack the form-factor, configurability, and energy resolution that would allow for high-specificity detection, short acquisition times, and differentiation of lesion size and depth in tissue (e.g. similar γ flux of large, deep and shallow, small lesions), respectively.

We bring advanced imaging, heretofore only accessible in a radiology suite, to the intraoperative environment by developing a fully integrated, hand-held intraoperative imager to guide surgeries (both cancer and other surgeries such as lymphatics surgeries) by enabling 3D imaging of tumor, lymphatics, and blood vessels, with increased sensitivity at depths beyond the range of conventional NIR/ICG-based imaging. In a single hand-held device, we accomplish this via simultaneous imaging of NIR fluorescence and radionuclide (SPECT) γ photons achieved by integrating 2 chip-scale imaging technologies-VISION and SENTRI, respectively. This fusion results in a multimodal intraoperative molecular imaging platform (VISION is a Versatile Imaging “Skin” for IntraOperative Navigation) integrated onto surgical instrumentation, capable of real-time microscopic molecular imaging at the tissue surface and deep in tissue (using NIR). This combined platform has the potential to significantly improve the outcomes of cancer surgery by identifying microscopic tumor deposits often left behind at the tumor margins and in lymph nodes. This combination achieves previously unattainable speed, sensitivity, and visualization at depth for molecular imaging in tissue-critical for identifying residual disease at the surgical margins and microscopic involvement of lymph nodes.

This platform uses a tumor-specific small molecule labeled with fluorophores and radiolabels and imaged with an integrated chip-scale sensor for both fluorescence and radiation detection, on a probe. The integration of SPECT sensing extends the depth imaging beyond the NIR range. This combination platform can be compatible with FDA approved agents, ICG and 99Tc colloids, we target primary (extremity) lymphedema and cancer (breast), or with custom imaging agents. In some instantiations, the same agent can be used with a NIR or other fluorescent dye, and co-injected with the same agent conjugated to a radionuclide (via a chelator), in other instances it can be a single molecule conjugated to both, and in other instances, it can be different molecules.

Impact: The ongoing inability to image microscopic tumor foci intraoperatively during prostatectomy results in a 20-40% rate of positive margins, driving a 2-4× increased risk of recurrence and significant additional treatment. Untreated lymph node metastases increase cancer recurrence, while unnecessary lymph node dissections cause morbidity. Intraoperative identification of microscopic tumor margins and lymph node metastases should enable removal of all disease to dramatically improve clinical outcomes in high-risk disease. Widely impactful, the VISION platform is applicable to any disease with a targetable marker. In the realm of lymphatic surgeries, this platform will improve patient outcomes by increasing the efficacy of, and decrease morbidity of, current lymphatic surgeries via enabling: (1) More effective intraoperative planning by accessing lymphatics beyond what is visible via ICG; (2) Simultaneous visualization of veins at depth, facilitating anastomosis/bypass, and (3) Real-time intraoperative assessment of function via monitoring imaging agent flow.

### Background: Tumor Imaging

No existing intraoperative imaging method enables high sensitivity imaging in real-time with an ultra-miniature, scalable, planar form-factor-necessary for broad device integration. Recent trials in fluorescence guided surgery have made significant progress¹ in prostate²⁻⁴, breast⁵⁻⁷, ovarian^{8,9}, head and neck^{10,11}, and lung¹², but remain limited to >~5 mm tumor deposits (100 million cells) at the tissue surface with even lower sensitivity for lymph nodes-buried under tissue. This stems from both imager sensitivity and the specificity and contrast of the labeling agent.

Intraoperative fluorescence imagers^{6,13} are largely based on the principle of placing a microscope above the tumor bed, reducing sensitivity due to large tissue to imager distance, or attached to a flexible fiber optic limiting the field of view. Sensitivity is also limited by background autofluorescence and photobleaching. Both optical (fluorescence^{6,14-19}, Raman spectroscopy²⁰⁻²³, Cerenkov²⁴⁻²⁶, optical coherence tomography²⁷⁻³⁰) and non-optical (e.g., radiotracers³¹⁻³⁵, mass spec³⁶) devices for cancer detection have been designed, but these platforms are not sufficiently maneuverable for hard to access areas. Unlike thin electronic wires carrying the image from a chip-based imager, coupling these devices to a fiber optic faces a fundamental trade-off between imaging area (diameter) and flexibility. Hence, many of these platforms are limited to imaging single points or select areas^{14,22,23,36-38}-unable to image the entire tumor bed in real-time.

To aid maneuverability, efforts at miniaturizing optics encounters have been attempted, but encounter several fundamental limitations such the significant aberration and imperfections present when fabricating optics at the microscale³⁹⁻⁴¹ and miniaturized fluorescence imagers are still several centimeters in length⁴²⁻⁴⁶. Contact image sensors eliminate these challenges by largely dispensing with optics in lieu of proximity to the tissue surface to achieve high sensitivity and the required spatial resolution, converting the optical image to an electronic one at the point of imaging. While these are used in in vitro applications⁴⁷⁻⁴⁹ where spatial resolution and speed requirements are relaxed, in vivo MRD detection requires real-time, high-sensitivity imaging and a new imaging platform technology for intraoperative imaging is needed.

The second limitation to sensitivity and depth imaging are the requisite optical agents. The growing armamentarium of molecularly-targeted imaging agents are now able to label individual tumor cells in patients 8,11,12,50-57, but light faces fundamental limits when penetrating tissue, losing signal strength and spatial resolution through scattering and absorption. In fluorescence, this problem is squared in magnitude, as light must penetrate to the labeled cell, and then the fluorescently emitted light must travel the same path back to the imager. This limits optical imaging to only a few millimeters, for all but large tumor deposits, and microscopic deposits remain undetectable at depth.

Another approach at intraoperative detection for lymph nodes relies on radiolabeled agents—otherwise known as radioguided surgery or RGS. These tumor labeling agents emit gamma photons, which readily penetrate tissue. Current drop-in gamma probes have surface areas limited by port size (~11 mm), hindering the ability to search over the entire operative field (tens of thousands of square mm). Instead, they are, in general, used to localize sentinel nodes with large signals (a 1-5 mCi injection largely accumulates in the node) and therefore relatively straightforward to identify. Non-specific markers encompassing both fluorescence and radiation (ICG-99mTc-nanocolloid) have been performed, but are still largely limited to the large signals generated common to sentinel lymph node mapping. Lack of probe surface area, sensitivity, and maneuverability constrain the ability to search the operative bed for low-signal emitting nodes. Probe maneuverability is constrained by the need for scintillating material and directional collimators which result in rigid probes several centimeters in length. In contrast, molecularly guided RGS relies on a systemically injected radiotracer, between 5 and 15 mCi of activity, of which only a small portion accumulates in the tumor involved node-making the signal much smaller. To mitigate this, tumor involved nodes need to be localized on preoperative SPECT/CT and then the probe is used to search in the region of interest intraoperatively. This creates significant logistic challenges, but also precludes the identification of nodes below the threshold of SPECT/CT or PSMA/PET, which is a few millimeters in diameter or roughly 100 million cells for a 5 mm tumor deposit. For example, 15 mCi of Tc99-PSMA-I&S was injected, and with 10% ID/g uptake, a 5 mm tumor deposit is 0.05 g, and would have an activity of 0.075 mCi (75 μCi). A 1 mm deposit would have activity of just 0.75 μCi. Current RGS systems used in clinical studies, such as SENSEI (Lighpoint Medical, now Telix), or Crystal Photonics, have sensitivities of ~1200 cps/MBq or ~1200 cps/27 μCi at 2 cm from the source. For small tumor deposits, not visible on preoperative imaging, a highly sensitive detector, with improved spatial resolution for RGS is needed to identify involved nodes beneath the tissue surface.

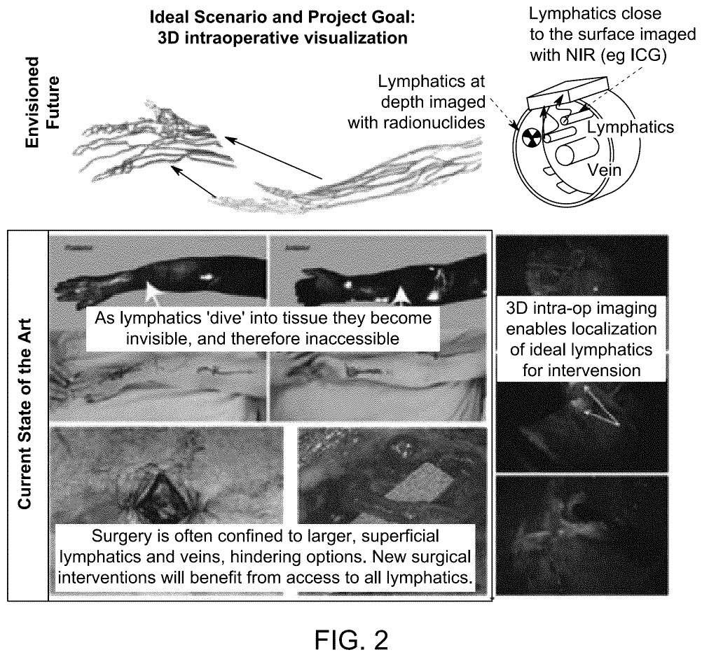

Application to lymphatic surgeries: Lymphatic surgeries represent both the mainstay of treatment (lymphovenous bypass, nodal transplant) and the future of lymphatic treatments (stents, implantable collagen scaffolds), yet imaging of lymphatics intraoperatively-both at the surface and at depth-remains an important, yet elusive, goal because of the inherent physical limits of both current imaging probes and their imagers. Intraoperative planning, guidance, and procedure assessment is currently hindered by the inability to see peripheral lymphatics significantly restricting the lymphatics that can be treated with surgery (FIG. 2). Patency, flow, and diameter of smaller lymphatics or those at depth cannot be assessed. Without detailed visualization, surgeons are either forced to treat only superficial and larger lymphatics or explore tissue, dissecting to reveal lymphatics, which is inefficient and introduces additional morbidity, versus a more planned approach where the location of the ideal target lymphatics are known. Given shifting anatomy during surgery, pre-operative imaging is insufficient and real-time imaging is needed. Moreover, multi-target imaging to visualize both lymphatics and veins-necessary for many bypass procedures-in unavailable at depth other dyes (fluorescein and methylene blue) have shorter wavelengths, resulting in even more limited depth penetration than ICG.

Intraoperative guidance is currently limited to ICG-based imaging^{58,59} (e.g. SPY), precluding use in larger patients or those with significant lymphedema, or critically when lymphatics dive deeper into tissue. Moreover, lymphatic bypass (the most used early surgical intervention) relies on identifying a neighboring vein, which is currently only identified by a shadow overlying the ICG illuminated lymphatic, severely limiting selection. Given these lymphatics and veins are millimeter scale at best, guiding surgeries below 1 cm tissue depth is currently infeasible.

No existing imaging method achieves detailed 3D imaging of intraoperative anatomy outside of the radiology suite and imaging platforms themselves remain the limiting reagent.

### The Optimal Intraoperative Lymphatic Imaging Solution Demands:

Detailed intraoperative imaging of lymphatics at the surface and at depth—beyond the capabilities of NIR dyes like ICG alone.

Integration of NIR fluorescence, for near surface imaging, and SPECT tracer imaging,
for near deeper lymphatic imaging, in a single, versatile, dual-modality hand-held platform to intraoperatively plan, guide, and assess surgery.
Simultaneous imaging of lymphatics and veins for lymphovenous anastomosis.
Simultaneous imaging of tumor lymphatics and surface margins.
Scalable imaging architecture, customizable to any anatomic disease site in a mass-produced computer-chip platform enabling low-cost at economies of scale.
Automated image processing and recognition to synthesize dual-modality imaging into a real-time 3D rendering, to enable rapid adoption in all care settings

### Chip-Scale Dual-Modality SPECT and NIR Imaging for 3D Lymphatic Intraoperative Imaging.

We introduce a new handheld lymphatic imaging approach integrating both NIR fluorescence imaging and SPECT imaging in a single ultrasensitive chip-scale γ-sensing platform⁶⁰⁻⁶³, compatible with custom imaging agents or off the self, FDA approved imaging agents (ICG, ⁹⁹Tc colloids). The SENTRI platform breaks the fundamental physical constraint of large, expensive, hospital-based SPECT imaging solutions in favor of a chip-scale, single γ photon detection with on-chip energy resolution without scintillators or collimators to maintain sub-millimeter thickness. We integrate our chip-scale SPECT platform with scalable architecture capable of single γ photon detection and energy resolution, with our chip-scale fluorescence NIR imaging platform, VISION⁶⁴⁻⁶⁸ by simply stacking these chips (as γ photons will pass through the VISION imager chip), creating a single 0.7×1×4 cm intraoperative imaging platform capable of simultaneous (this size can be customized using our scalable platform to really any size, depending on the application), synergistic, imaging of these two modalities. Computer-vision based approaches integrate these two datasets to allow a seamlessly integrated 3D intraoperative map of lymphatic vessels for real-time surgical guidance.

Computer-vision based approaches integrate these two datasets to allow a seamlessly integrated 3D intraoperative map of lymphatic vessels for real-time surgical guidance. Multicolor fluorescence imaging and energy resolution allow for multiple fluorophores and radionuclides to be used, opening the door to simultaneous imaging of both lymphatics and veins.

(1) Chip-Scale Single γ Photon Sensors Made from Integrated Circuit (IC) Technology
An array of ultrasmall (≤1 μm²) on-chip sensing pixels, with in-pixel amplification produces a large signal per γ photon
Made from ‘computer chip’ technology, the platform is inherently modular and scalable in size
Trillion-$ ongoing investments in IC fabs enable low-cost mass production and wide accessibility.

### (2) a Novel Energy-Resolving Sensor Design Enables Depth Sensing and Simultaneous Multi-Radionuclide Imaging

On-chip, in-pixel circuitry enables relative quantification of γ photon energies, distinguishing different radionuclides and inferring γ source depth.

### (3) Thin Chip-Scale Sensors Enable Stacking of SENTRI and VISION in a Single, Compact, Handheld Device

γ photons will pass through VISION and detected by SENTRI, enabling dual-modality imaging in a single hand-held device

### (4) Computer Vision Enabled 3D Imaging

Integrating VISION NIR fluorescence and SENTRI cm-scale depth imaging for real time 3D intraoperative mapping.

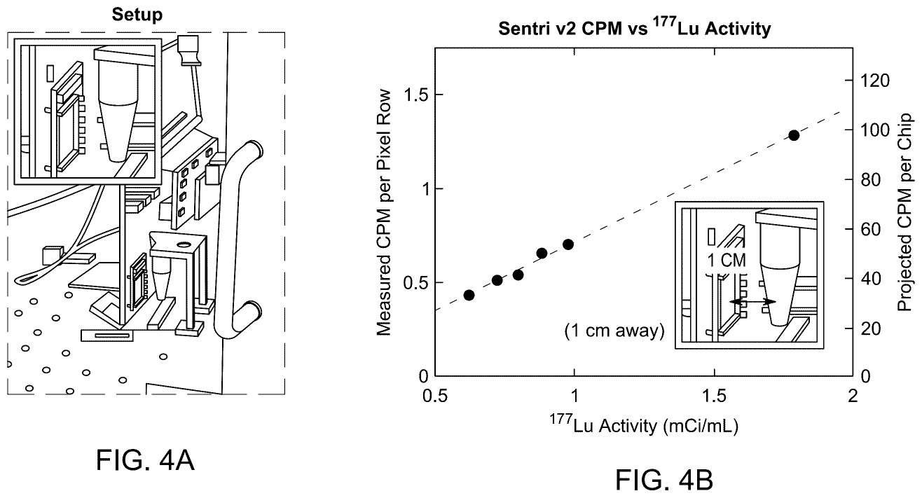

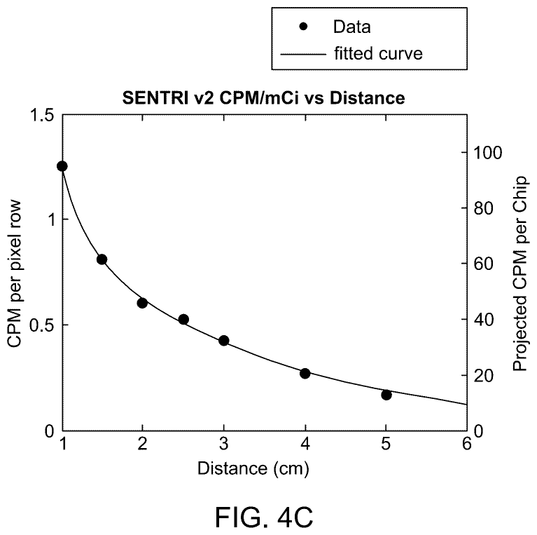

Sensitivity is enhanced while maintaining sensor flux via a massively paralleled-array of ultra-small sensors integrated with in-pixel application, on a single chiplet. Building on our chip-scale energy-resolving γ photon sensing platform (data shown in FIGS. 3 and 4), SENTRI utilizes ultrasmall (<1 μm²) on-chip.

Integration of SENTRI with NIR-II Fluorescence Imaging for Deep Intraoperative Imaging

Leveraging the inherent scalability and event-driven readout architecture of our 3×3 mm² design, we scale SENTRI to ~1×4 cm² to match with our VISION NIR imager, placing the chips back-to-back (we can optimize for really any size). We optimize display software to seamlessly integrate NIR and SPECT imaging information. Once complete, we then explore additional approaches to further delineate lymphatics in 3 dimensions. First, we harness the information from the energy distribution of the γ photons—as some γ photons emerging from deeper in tissue (e.g., cm-scale depths) will (Compton) scatter and lose energy. Thus, the ability of SENTRI to measure energy resolution can infer the depth. Additional efforts at integrating directionality on chip (such as stacking 2 SENTRI chips back-to-back and determining the angle of γ incidence) can further enhance depth resolution. SENTRI patches can also be placed on skin to triangulate the radionuclide source in 3D.

As proof of concept, we have demonstrated this approach in a mouse model of targeted radionuclides and external gamma photon probes, whereby radionuclides are injected and localize to tumors, kidney, and bladder. We use a priori knowledge of the radionuclide localization (i.e., via preoperative MRI), to significantly reduce the amount of information needed for reconstruction-enabling sampling of γs at discrete locations. Our results are compared with small animal SPECT/CT and show high concordance.

Dual-modality nanoparticles In addition to small molecules or antibodies, we can also use nanoparticles that contain both fluorophores and radionuclides.

### Improvements in VISION for Deeper NIR Imaging

Using InGaAs imaging chips for deeper NIR imaging
Using deep deleted silicon imagers for better responsivity in the 800-1000 nm range

### Improvements in SENTRI for Improved Energy Resolution (and Thereby Depth Assessment)

Custom ACIC: On instantiation of SENTRI for energy resolution is a γ spectrometer ASIC in 180 nm CMOS that (1) is mm-scale, pixelated, and single γ sensitive to allow for high-specificity detection, (2) has pixels that can be configured individually based on the incoming γ flux and PVT variations to maximize detection area and minimize acquisition times, and (3) has an energy resolving scheme with configurable resolution and dynamic range to encode lesion depth in tissue (i.e. energy profiles of γ's change with cm-scale tissue depth due to scattering [13]). Previous works [14]-[17] have shown promise in achieving energy-binning of radiation for dosimetry under high-flux X-ray beams, but suffer from few energy bins, low detection area per energy bin, and no configurability, incompatible with low-flux, wide-energy γ-sensing applications such as RGS.

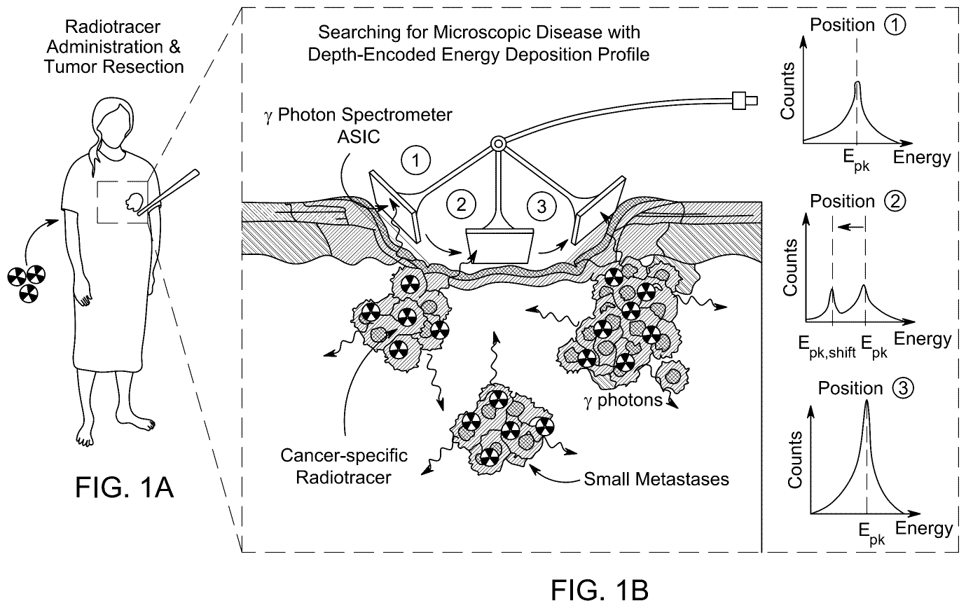

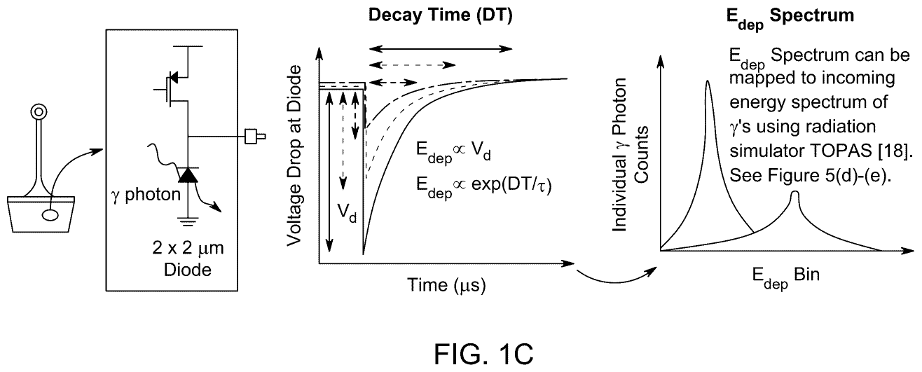

As an example of the envisioned workflow, FIG. 1(a) depicts a patient that presents with an early-stage tumor, is administered a cancer-specific radiotracer, and undergoes surgical tumor resection. FIG. 1(b) depicts the movement of the γ spectrometer ASIC around the resection site to search for additional cancerous lesions. At each position, the ASIC encodes both the depth information and lesion size in its measured energy spectrum in unique ways. FIG. 1(c) demonstrates the individual γ sensing and energy-resolving methodology using 2×2 μm reverse-biased diodes. γ's are difficult to directly sense because they are sparsely ionizing, but since small diodes have small parasitic capacitances (C_{diode,par}), a miniscule amount of charge deposition (Q_{gen}) generates a large voltage drop (V_{d}=Q_{gen}/C_{diode,par}) at the diode node. Va is proportional to the energy deposited (E_{dep}), and because the diode node has an RC time constant (T), the E_{dep} is also related to the time it takes for the voltage signal to decay back (decay time, ‘DT’) to DC. We can measure DT in the time domain and leverage that E_{dep} & exp (DT/T) to resolve E_{dep}, allowing all pixels to measure energy deposition without the need for large area or high power ADCs. The measured E_{dep} spectrum can be mapped to the incident γ energy profile using the gold-standard radiation simulation software, TOPAS (GEANT4) [18].

Because RGS requires scattered photon detection for depth-encoding, small acquisition times for rapid scanning of the resection site, and ideal energy resolution and dynamic range, this system has 3 diode-based pixel architectures that optimize for fine energy-resolution (FIG. 2(a)), low-flux applications (FIG. 2(b)), and energy-calibration (FIG. 2(c)), respectively. When a γ interacts with the depletion region of a reverse-biased diode, there is a small drop in voltage at the diode node. To reduce diode capacitive loading, a unity-gain OTA with a minimum sized input-pair is utilized in every pixel. In the energy-resolving pixel, the output of this buffer is connected to an OTA with a DC compensation loop that secures the output DC level of this OTA. This output is routed to a PMOS common-source (CS) stage with configurable load to set the custom sensitivity of the pixel. The load is configured with 3-bit in-pixel 6T SRAM that is used to control in-pixel DACs that set the gate voltage of the CS active load. The pulse is digitized using two inverters and sent to the digital DT processing unit via a set of switches, where the width of these digital pulses are measured to estimate the DT. In the low-flux pixel, 6 of these reverse-biased diodes and unity-gain buffer pairs are utilized to maximize fill-factor, and the outputs are connected together. This output is routed to the input of an OTA that has an identical unity-gain buffer in its feedback loop, serving as a small-area pseudoresistor to create a summing amplifier with gain=−1. The summation of individual γ hits from any of the 6 diodes is routed to an open-loop OTA with a DC compensation loop, NMOS CS-stage with SRAM-configurable load, and inverters for count digitization similar to the energy-resolving pixel. Low-flux pixels have 2× the area, 6× the diode area, and 3× the fill-factor while maintaining the same diode sensitivity. Energy-calibrating pixels are similar to energy-resolving pixels but with varying diode sizes (4.41, 4.84, 5.29, 5.76, or 6.25 μm²), increasing C_{diode,par} and decreasing Va proportionally. This enables coarse energy-binning to find the maximum E_{dep} by a γ-emission, and based on this, the LSB used for DT estimation in the time domain is chosen to optimally tradeoff energy resolution and dynamic range. FIG. 2(d) shows the OTA and switches used in all pixels.

FIG. 3 shows the γ spectrometer's system architecture, including the configurable pixel array, DT processing unit, and SRAM sensitivity calibration unit. 61 rows of the pixel array use the energy-resolving pixel, 10 rows use the low-flux pixel, and 5 rows use the energy-calibrating pixels. Pixels in rows 72-76 have 6.25, 5.76, 5.29, 4.84, and 4.41 μm² diodes, respectively. Each row shares a count-line (CL) and count column line (CCL). The CL outputs a high pulse whenever a γ has been detected. Non-overlapping column address sweep (CAS) signals are routed down every column of the pixel array. Whenever a CAS signal is high and a γ is detected, CCL outputs a low pulse to identify the column address. CL and CCL are routed to the DT processing unit. There, the number of cycles CL is high is measured with a 10-bit counter that increases its value based on a configurable period clock (1-128 μs) for adjustable energy resolution. The DT, column, and row data are priority encoded, queued into a 26×16 FIFO, serialized, and sent to an FPGA. The SRAM sensitivity calibration unit is used to read/write to the 3-bit in-pixel SRAM to control the gain of each CS-stage. Each pixel is initially calibrated with no radiation present by enabling one pixel at a time and checking the CL of the corresponding row using a 76×1 row multiplexer. The SRAM value is increased from 0-7 to boost the CS gain until the CL is triggered by noise, and the SRAM is set to one value below this threshold.

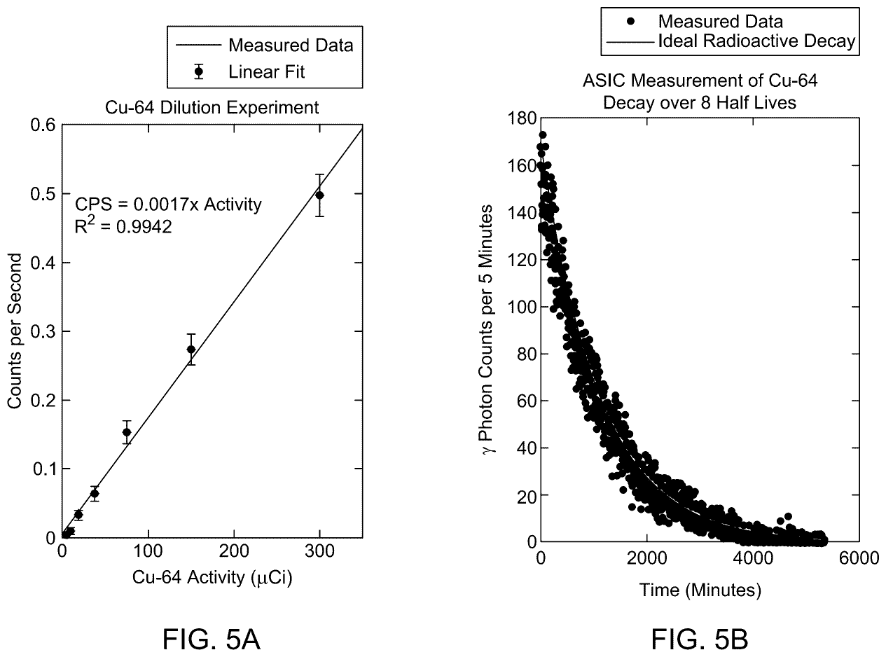

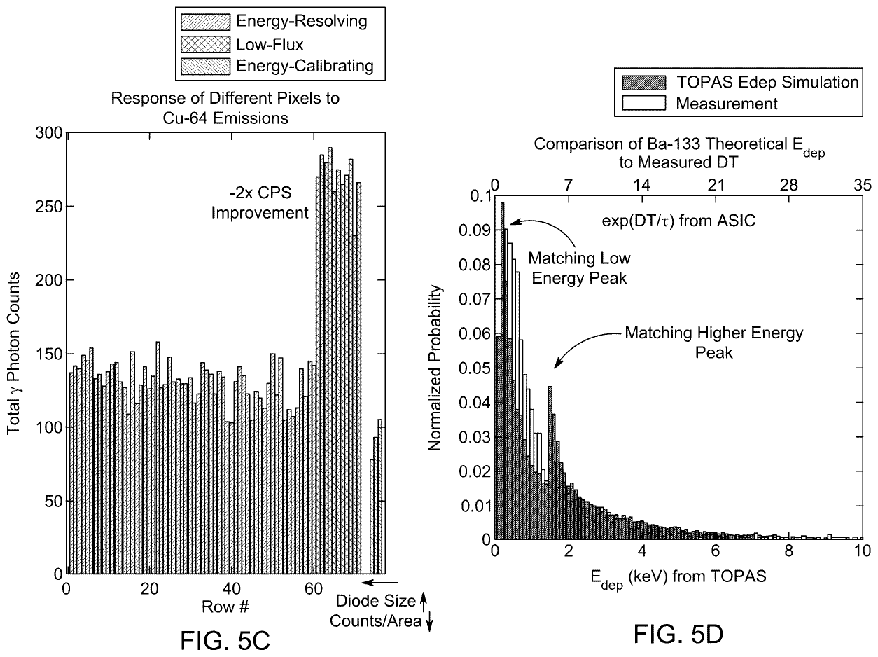

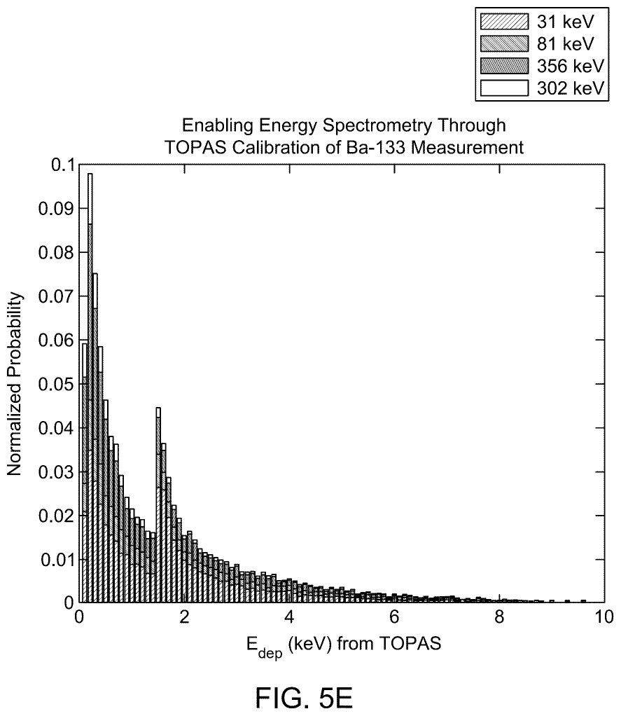

FIGS. 4(a) and (b) show a diagram and photo of the experimental setup used to test the γ spectrometer. The ASIC and daughterboard are mounted on one end of a linear stage. As a proof-of-concept, a γ-emitting point source of Cu-64 (512, 1346 keV emissions) or Ba-133 (31, 81, 302, 356 keV emissions) was placed 1 cm away from the ASIC. First, 7 vials with 5 μL of Cu-64 (300, 150, 75, 38, 19, 9, 4.5 μCi) were prepared and measured. The ASIC's counts per second (CPS) were highly linear with radioactive activity (R²=0.9942) (FIG. 5(a)). The ASIC was then used to continually monitor a vial with 300 μCi of Cu-64 (half-life=12.7 hours) in 5 μL for 105 hours. Acquisitions were taken every 5 minutes and compared to Cu-64's ideal radioactive decay curve. The system accurately tracks γ-flux activity levels as low as 1 μCi with 5 minute recordings (FIG. 5(b)). FIG. 5(c) reports the response of each pixel row to Cu-64. Due to the in-pixel sensitivity calibration, all the energy-resolving pixel rows have similar counts. The low-flux pixel rows have 2× higher counts compared to the energy-resolving pixels given their increased fill-factor but are <3× higher due to reduced SNR at the current summation node. The counts in the energy-calibrating pixel rows decrease with increasing diode size, with the 5.76 and 6.25 μm² diode rows recording 0 counts. This helps deduce the maximum E_{dep} from Cu-64, allowing DT LSB calibration to prevent saturating the 10-bit counter. Due to its many γ energies, a 1 μCi Ba-133 source was used to demonstrate the ASIC's ability to encode E_{dep} with DT. E_{dep} in the diode depletion region by Ba-133 was simulated using TOPAS. The simulated E_{dep} with sub-keV energy bins matched well with the measured quantity from the ASIC, exp (DT/T), as shown in FIG. 5(d). Because simulated E_{dep} can be labelled by the incident γ energy that deposited it, the simulation can be used to identify the incident γ energy spectrum in the measured exp (DT/T) distribution (FIG. 5(e)).

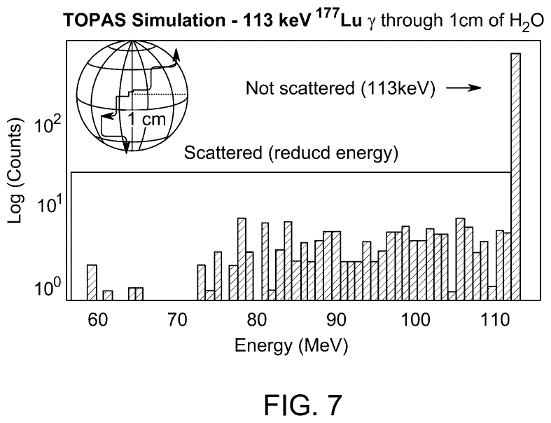

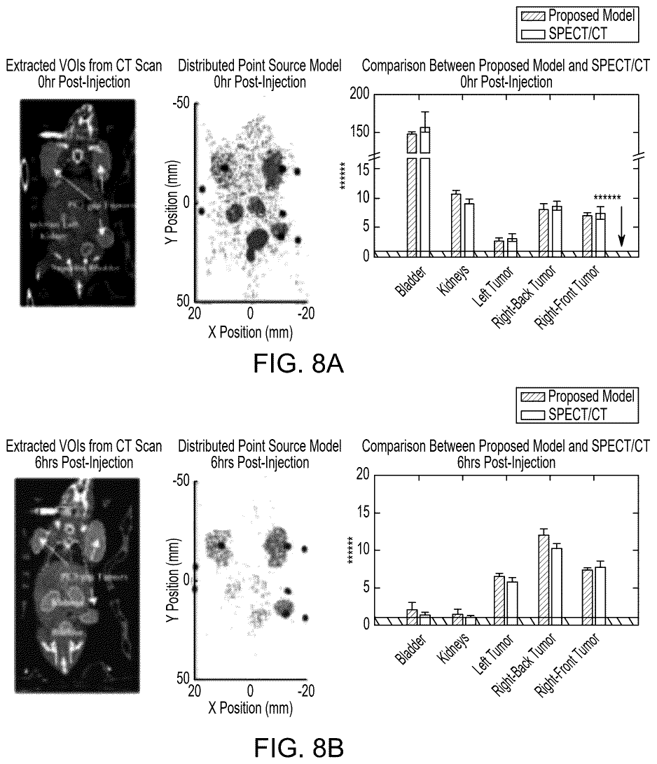

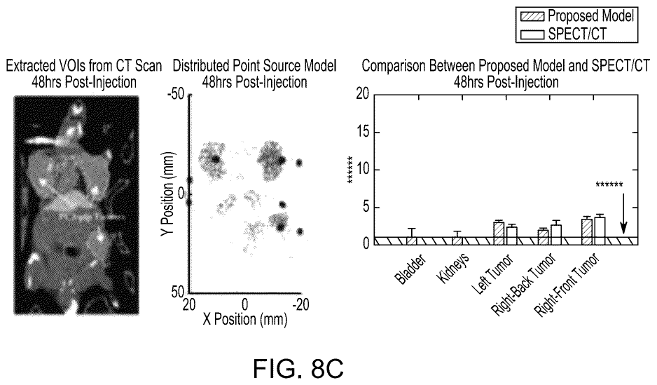

FIG. 6 is a comparison table with state-of-the-art radiation detectors [14]-[17] and FIG. 7 is an annotated die photo. This ASIC demonstrates the widest energy detection range (31-1346 keV), lowest resolvable radioactive activity (1 μCi), lowest total static power consumption (0.6 mW), and is the only system with configurable sensitivity (8 in-pixel levels) and energy resolution (LSB 1-128 μs), allowing for low-flux γ-sensing. We envision this ASIC will enable precision RGS to improve surgical outcomes in cancer.

Other Options: Using a CCD with scintillator material on top, with or without a fiber optic plate.

### REFERENCES

1. Azari, F. et al. Intraoperative molecular imaging clinical trials: a review of 2020 conference proceedings. J Biomed Opt 26, (2021).
2. Nguyen, H. Intraoperative PSMA florescence: guided, robotic radical prostatectomy: results of a phase one study. in Molecular-Guided Surgery: Molecules, Devices, and Applications VIII (eds. Gibbs, S. L., Pogue, B. W. & Gioux, S.) 36 (SPIE, 2022). doi: 10.1117/12.2619285.
3. Nguyen, H. et al. PD61-04 PRELIMINARY PHASE 1 SAFETY AND EFFICACY RESULTS OF A PROSTATE SPECIFIC MEMBRANE ANTIGEN (PSMA) TARGETING FLUOROPHORE IN PATIENTS UNDERGOING ROBOTIC PROSTATECTOMY. Journal of Urology 206, (2021).
4. Nguyen, H. et al. PD54-11 RESULTS OF THE PHASE 1 SAFETY AND EFFICACY PROSTATE SPECIFIC MEMBRANE ANTIGEN (PSMA) TARGETING FLUOROPHORE FOR IMAGE GUIDED SURGERY IN PATIENTS UNDERGOING ROBOTIC PROSTATECTOMY. Journal of Urology 207, (2022).
5. Keating, J. et al. Identification of breast cancer margins using intraoperative near-infrared imaging. J Surg Oncol 113, 508-514 (2016).
6. Troyan, S. L. et al. The FLARE intraoperative near-infrared fluorescence imaging system: a first-in-human clinical trial in breast cancer sentinel lymph node mapping. Ann Surg Oncol 16, 2943-2952 (2009).
7 Ottolino-Perry, K. et al. Intraoperative fluorescence imaging with aminolevulinic acid detects grossly occult breast cancer: a phase II randomized controlled trial. Breast Cancer Research 23, 72 (2021).
8. Tanyi, J. L. et al. A Phase III Study of Pafolacianine Injection (OTL38) for Intraoperative Imaging of Folate Receptor-Positive Ovarian Cancer (Study 006). Journal of Clinical Oncology (2022) doi: 10.1200/JCO.22.00291.
9. van Dam, G. M. et al. Intraoperative tumor-specific fluorescence imaging in ovarian cancer by folate receptor-[alpha] targeting: first in-human results. Nat Med 17, 1315-1319 (2011).
10. Rosenthal, E. L. et al. Safety and Tumor Specificity of Cetuximab-IRDye800 for Surgical Navigation in Head and Neck Cancer. Clinical Cancer Research 21, 3658-3666 (2015).
11. Gao, R. W. et al. Safety of panitumumab-IRDye800CW and cetuximab-IRDye800CW for fluorescence-guided surgical navigation in head and neck cancers. Theranostics 8, 2488-2495 (2018).
12. Gangadharan, S. et al. Multiinstitutional Phase 2 Clinical Trial of Intraoperative Molecular Imaging of Lung Cancer. Ann Thorac Surg 112, 1150-1159 (2021).
13. Zhao, Q. et al. A handheld fluorescence molecular tomography system for intraoperative optical imaging of tumor margins. Med Phys 38, 5873-5878 (2011).
14. Yoon, Y., Mohs, A. M., Mancini, M. C., Nie, S. & Shim, H. Combination of an Integrin-Targeting NIR Tracer and an Ultrasensitive Spectroscopic Device for Intraoperative Detection of Head and Neck Tumor Margins and Metastatic Lymph Nodes. Tomography: a journal for imaging research 2, 215-222 (2016).
15. Harmsen, S., Teraphongphom, N., Tweedle, M. F., Basilion, J. P. & Rosenthal, E. L. Optical surgical navigation for precision in tumor resections. Molecular imaging and biology: MIB: the official publication of the Academy of Molecular Imaging 19, 357-362 (2017).
16. Nishio, N. et al. Optimal Dosing Strategy for Fluorescence-Guided Surgery with Panitumumab-IRDye800CW in Head and Neck Cancer. Molecular imaging and biology: MIB: the official publication of the Academy of Molecular Imaging 22, 156-164 (2020).
17. de Boer, E. et al. Biodistribution Study of Intravenously Injected Cetuximab-IRDye700DX in Cynomolgus Macaques. Molecular imaging and biology: MIB: the official publication of the Academy of Molecular Imaging 18, 232-242 (2016).
18. Miller, S. E. et al. First-in-human intraoperative near-infrared fluorescence imaging of glioblastoma using cetuximab-IRDye800. J Neurooncol 139, 135-143 (2018).
19. Shin, D., Pierce, M. C., Gillenwater, A. M., Williams, M. D. & Richards-Kortum, R. R. A fiber-optic fluorescence microscope using a consumer-grade digital camera for in vivo cellular imaging. PLOS One 5, e11218 (2010).
20. Abramczyk, H. & Brozek-Pluska, B. Raman imaging in biochemical and biomedical applications. Diagnosis and treatment of breast cancer. Chem Rev 113, 5766-5781 (2013).
21. Birtoiu, I. A. et al. Diagnosing clean margins through Raman spectroscopy in human and animal mammary tumour surgery: a short review. Interface Focus 6, 20160067 (2016).
22. Mohs, A. M. et al. Hand-held spectroscopic device for in vivo and intraoperative tumor detection: contrast enhancement, detection sensitivity, and tissue penetration. Anal Chem 82, 9058-9065 (2010).
23. Jermyn, M. et al. Intraoperative brain cancer detection with Raman spectroscopy in

humans. Sci Transl Med 7, 274ra19 (2015).
24. Tanha, K., Pashazadeh, A. M. & Pogue, B. W. Review of biomedical Čerenkov luminescence imaging applications. Biomed Opt Express 6, 3053-3065 (2015).
25. Grootendorst, M. R. et al. Intraoperative Assessment of Tumor Resection Margins in Breast-Conserving Surgery Using 18F-FDG Cerenkov Luminescence Imaging: A First-in-Human Feasibility Study. Journal of Nuclear Medicine 58, 891-898 (2017).
26. Nakamura, Y. et al. Cerenkov radiation-induced photoimmunotherapy with (18) F-FDG. Journal of Nuclear Medicine (2017) doi: 10.2967/jnumed. 116.188789.
27. Nguyen, F. T. et al. Intraoperative evaluation of breast tumor margins with optical coherence tomography. Cancer Res 69, 8790-8796 (2009).
28. Zysk, A. M. et al. Intraoperative Assessment of Final Margins with a Handheld Optical Imaging Probe During Breast-Conserving Surgery May Reduce the Reoperation Rate: Results of a Multicenter Study. Ann Surg Oncol 22, 3356-3362 (2015).
29. Carrasco-Zevallos, O. M. et al. Review of intraoperative optical coherence tomography: technology and applications [Invited]. Biomed Opt Express 8, 1607 (2017).
30. Muijzer, M. B. et al. Clinical applications for intraoperative optical coherence tomography: a systematic review. Eye 36, 379-391 (2022).
31. Türkcan, S. et al. Endoscopic detection of cancer with lensless radioluminescence imaging and machine vision. Sci Rep 6, 30737 (2016).
32. Sarikaya, I. et al. Combined use of preoperative 18F FDG-PET imaging and intraoperative gamma probe detection for accurate assessment of tumor recurrence in patients with colorectal cancer. World J Surg Oncol 5, 80 (2007).
33. Gulec, S. A. PET probe-guided surgery. J Surg Oncol 96, 353-357 (2007).
34. Rauscher, I., Horn, T., Eiber, M., Gschwend, J. E. & Maurer, T. Novel technology of molecular radio-guidance for lymph node dissection in recurrent prostate cancer by PSMA-ligands. World J Urol 36, 603-608 (2018).
35. de Barros, H. A. et al. Robot-assisted Prostate-specific Membrane Antigen-radioguided Salvage Surgery in Recurrent Prostate Cancer Using a DROP-IN Gamma Probe: The First Prospective Feasibility Study. Eur Urol 82, 97-105 (2022).
36. Zhang, J. et al. Nondestructive tissue analysis for ex vivo and in vivo cancer diagnosis using a handheld mass spectrometry system. Sci Transl Med 9, (2017).
37. Villiger, M. et al. Deep tissue volume imaging of birefringence through fibre-optic needle probes for the delineation of breast tumour. Sci Rep 6, 28771 (2016).
38. Cousins, A. et al. Novel handheld magnetometer probe based on magnetic tunnelling junction sensors for intraoperative sentinel lymph node identification. Sci Rep 5, 10842 (2015).
39. Fletcher, D. A. et al. Near-field infrared imaging with a microfabricated solid immersion lens. Appl Phys Lett 77, 2109-2111 (2000).
40. Brady, D. J. & Hagen, N. Multiscale lens design. Opt Express 17, 10659-10674 (2009).
41. Wu, D. et al. Bioinspired Fabrication of High-Quality 3D Artificial Compound Eyes by Voxel-Modulation Femtosecond Laser Writing for Distortion-Free Wide-Field-of-View Imaging. Adv Opt Mater 2, 751-758 (2014).
42. Gambin, Y., Legrand, O. & Quake, S. R. Microfabricated rubber microscope using soft solid immersion lenses. Appl Phys Lett 88, 174102 (2006).
43. Flusberg, B. A. et al. High-speed, miniaturized fluorescence microscopy in freely moving mice. Nat Methods 5, 935-938 (2008).
44. Ghosh, K. K. et al. Miniaturized integration of a fluorescence microscope. Nat Methods 8, 871-878 (2011).
45. Breslauer, D. N., Maamari, R. N., Switz, N. A., Lam, W. A. & Fletcher, D. A. Mobile phone based clinical microscopy for global health applications. PLOS One 4, e6320 (2009).
46. Wei, Q. et al. Fluorescent imaging of single nanoparticles and viruses on a smart phone. ACS Nano 7, 9147-9155 (2013).
47. Singh, R. R., Leng, L., Guenther, A. & Genov, R. A CMOS-Microfluidic Chemiluminescence Contact Imaging Microsystem. IEEE J Solid-State Circuits 47, 2822-2833 (2012).
48. Guo, N., Cheung, K., Wong, H. T. & Ho, D. CMOS time-resolved, contact, and multispectral fluorescence imaging for DNA molecular diagnostics. Sensors (Basel) 14, 20602-20619 (2014).
49. Singh, R. R. et al. A CMOS/Thin-Film Fluorescence Contact Imaging Microsystem for DNA Analysis. IEEE Transactions on Circuits and Systems I: Regular Papers 57, 1029-1038 (2010).
50. Jiang, T. et al. Tumor imaging by means of proteolytic activation of cell-penetrating peptides. Proc Natl Acad Sci USA 101, 17867-17872 (2004).
51. Whitney, M. A. et al. Fluorescent peptides highlight peripheral nerves during surgery in mice. Nat Biotechnol 29, 352-356 (2011).
52. Fidel, J. et al. Preclinical Validation of the Utility of BLZ-100 in Providing Fluorescence Contrast for Imaging Spontaneous Solid Tumors. Cancer Res 75, 4283-4291 (2015).
53. Butte, P. v et al. Near-infrared imaging of brain tumors using the Tumor Paint BLZ-100 to achieve near-complete resection of brain tumors. Neurosurg Focus 36, E1 (2014).
54. Ogawa, M., Kosaka, N., Choyke, P. L. & Kobayashi, H. In vivo molecular imaging of cancer with a quenching near-infrared fluorescent probe using conjugates of monoclonal antibodies and indocyanine green. Cancer Res 69, 1268-1272 (2009).
55. Alander, J. T. et al. A review of indocyanine green fluorescent imaging in surgery. Int J Biomed Imaging 2012, 940585 (2012).
56. Mitsunaga, M. et al. Cancer cell-selective in vivo near infrared photoimmunotherapy targeting specific membrane molecules. Nat Med 17, 1685-1691 (2011).
57. Koch, M. et al. Threshold analysis and biodistribution of fluorescently labeled bevacizumab in human breast cancer. Cancer Res 77, 623-631 (2017).
58. Liu, H.-L., Pang, S.-Y. & Chan, Y.-W. The use of a microscope with near-infrared imaging function in indocyanine green lymphography and lymphaticovenous anastomosis. Journal of Plastic, Reconstructive & Aesthetic Surgery 67, 231-236 (2014).
59. Burnier, P., Niddam, J., Bosc, R., Hersant, B. & Meningaud, J.-P. Indocyanine green applications in plastic surgery: A review of the literature. Journal of Plastic, Reconstructive & Aesthetic Surgery 70, 814-827 (2017).
60. Lee, K. et al. SENTRI: Single-Particle Energy Transducer for Radionuclide Injections for Personalized Targeted Radionuclide Cancer Therapy. International Journal of Radiation Oncology*Biology*Physics (2023) doi: 10.1016/j.ijrobp.2023.11.057.
61. Lee, K. et al. Personalized Lu177 Theranostic Dosimetry Using a Single Beta-Particle In Vivo Microdosimeter. International Journal of Radiation Oncology*Biology*Physics 114, S125 (2022).
62. Lall, R., Seo, Y., Niknejad, A. & Anwar, M. A 3×3.3 mm Configurable γ Photon Spectrometer for Precision Radioguided Cancer Resection (Accepted and Demo). in 2025 IEEE International Solid State Circuits Conference (ISSCC) (IEEE, San Francisco, 2025).
63. Lall, R. et al. A 76×55 X-Ray Energy Binning Dosimeter for Closed-Loop Cancer Radiotherapy. in 2024 IEEE Symposium on VLSI Technology and Circuits (VLSI Technology and Circuits) 1-2 (IEEE, 2024). doi: 10.1109/VLSITechnologyandCir46783.2024.10631452.
64. Roschelle, M. et al. Multicolor fluorescence microscopy for surgical guidance using a chip-scale imager with a low-NA fiber optic plate and a multi-bandpass interference filter. Biomed Opt Express 15, 1761 (2024).
65. Papageorgiou, E. P., Boser, B. & Anwar, M. Chip-Scale Angle-Selective Imager for In

Vivo Microscopic Cancer Detection. IEEE Trans Biomed Circuits Syst (2019) doi: 10.1109/TBCAS.2019.2959278.
66. Wong, J., Papageorgiou, E., Giverts, S., Park, C. & Anwar, M. Feasibility of Breast Tumor Imaging in a Mouse Model Using Angle Selective Fluorescence Contact Imaging. in ANNALS OF SURGICAL ONCOLOGY S91-S91 (SPRINGER 233 SPRING ST, NEWYORK, NY 10013 USA, 2019).
67. Papageorgiou, E. P., Zhang, H., Boser, B. E., Park, C. & Anwar, M. Angle-insensitive amorphous silicon optical filter for fluorescence contact imaging. Opt Lett 43, 354-357 (2018).
68. Papageorgiou, E. P. et al. Real-time cancer detection with an integrated lensless fluorescence contact imager. Biomed Opt Express 9, 3607-3623 (2018).

TABLE 1
Comparisons SENTRI is integrated w/VISION into dual modality intraop imaging

| | | Intra- | | | | | | | Flow |
|---|---|---|---|---|---|---|---|---|---|
| Modality | FOV | Op | Depth | | Time | $$$ | Res | | Rates |
| ICG (e.g. SPY) | Region | Yes | ~1 | cm | seconds | $0.5-1M | mm | | − |
| Lymphangio- | Region | No | | | 3-5 hrs | $1M | >5 | mm | − |
| scintigraphy | | | | | | | | | |
| CT lymphan- | Body | No | | | 3-5 hrs | $1-3M | 1-5 | mm | − |
| giography | | | | | | | | | |
| DCMRL | Body | No | | | 3-5 hrs | $3-6M | ~1 | mm | +/− |
| SENTRI | Organ | yes | 1-5 | cm | <1 s/ | <$3,000 | <200 | μm | Yes |
| (+VISION) | | | | | image | | | | |

1. A dual modality imager comprising:
a first chip comprising an array of gamma photon-sensing pixels, wherein each gamma photon-sensing pixel provides in-pixel signal amplification for incoming gamma photons that hit the gamma photon-sensing pixel and in-pixel data processing to determine relative energies and angles of incidence of the incoming gamma photons, and wherein each gamma photon-sensing pixel only outputs data from the in-pixel data processing in response to an incoming gamma photon hitting the gamma photon-sensing pixel; and
a second chip comprising an array of fluorescence-sensing pixels, wherein the second chip is positioned relative to the first chip such that the incoming gamma photons pass through the second chip to reach the array of gamma photon-sensing pixels.

2. The dual modality imager of claim 1, wherein the dual modality imager is configured to perform simultaneous imaging of gamma photons and fluorescence.

3. The dual modality imager of claim 1, wherein the dual modality imager is configured to perform intraoperative image-guided surgery.

4. The dual modality imager of claim 3, wherein the in-pixel data processing further determines tissue depths of gamma-emitting radionuclide sources from which the incoming gamma photons originate.

5. The dual modality imager of claim 1, wherein the imaging of gamma photons is single photon emission computed tomography (SPECT) imaging.

6. The dual modality imager of claim 1, wherein the fluorescence-sensing pixels detect fluorescent light in a near-infrared or visible region of the electromagnetic spectrum.

7. The dual modality imager of claim 1, wherein the dual modality imager further comprises a handle extending away and disposed angularly from the first chip.

8. The dual modality imager of claim 1, wherein each gamma photon-sensing pixel comprises at least one silicon diode, wherein a gamma photon incident on a surface of the at least one silicon diode breaks a silicon bond to produce an electron-hole pair, wherein a pulse of charge (Qₚ) is generated that is accumulated by a diode capacitor resulting in generation of a voltage pulse.

9-17. (canceled)

18. The dual modality imager of claim 1, wherein the first chip is covered with a Compton scattering material.

19-20. (canceled)

21. The dual modality imager of claim 1, wherein the first chip further comprises on-chip circuitry tuned to be responsive to a range of linear energy transfer (LET) from incoming gamma photons such that combination and distribution of signals from gamma photon-sensing pixels with known LET responsivity enables determination of incident energies of the incoming gamma photons.

22-23. (canceled)

24. The dual modality imager of claim 1, wherein each fluorescence-sensing pixel comprises a photodiode, wherein florescent fluorescent light incident on a surface of the photodiode is converted into an electrical current proportional to intensity of the incident fluorescent light.

25. (canceled)

26. The dual modality imager of claim 1, wherein the array of fluorescence-sensing pixels comprises a plurality of photodiodes arrayed on the surface of the second chip, wherein each photodiode is coated with at least one layer of filter material that functions as an optical filter.

27. The dual modality imager of claim 1, wherein the second chip further comprises:
a plurality of light emitting sources to provide excitation light, wherein the plurality of light emitting sources are located on the second chip or externally;
an energy storage device to supply power for operation of the second chip, wherein the energy storage device is located on the second chip or externally;
a data storage unit in communication with the array of fluorescence-sensing pixels, wherein the data storage unit stores imaging data from the array of fluorescence-sensing pixels; and
an application-specific integrated circuit (ASIC) configured to control voltages and supply power from the energy storage device to the array of fluorescence-sensing pixels, the plurality of light emitting sources on the second chip, and the data storage unit.

28-53. (canceled)

54. A system comprising:
a dual modality imager, comprising:
a first chip comprising an array of gamma photon-sensing pixels, wherein each gamma photon-sensing pixel provides in-pixel signal amplification for incoming gamma photons that hit the gamma photon-sensing pixel and in-pixel data processing to determine relative energies and angles of incidence of the incoming gamma photons, and wherein each gamma photon-sensing pixel only outputs data from the in-pixel data processing in response to an incoming gamma photon hitting the gamma photon-sensing pixel; and
a second chip comprising an array of fluorescence-sensing pixels, wherein the second chip is positioned relative to the first chip such that the incoming gamma photons pass through the second chip to reach the array of gamma photon-sensing pixels;
a processor;
an external data receiving device connected to the processor, wherein the external data receiving device receives imaging data from the array of gamma photon-sensing pixels and the array of fluorescence-sensing pixels of the dual modality imager; and
a display component configured to display an image showing the fluorescence detected by the fluorescence-sensing pixels and the gamma photons detected by the gamma photon-sensing pixels.

55. The system of claim 54, further comprising an imaging agent comprising a fluorophore and a gamma-emitting radionuclide conjugated to a binding agent that selectively binds to a cellular marker.

56. The system of claim 55, wherein the cellular marker is a tumor marker.

57. The system of claim 55, wherein the cellular marker is a disease-associated marker.

58-60. (canceled)

61. The system of claim 54, further comprising a first imaging agent comprising a fluorophore conjugated to a first binding agent that selectively binds to a first cellular marker and a second imaging agent comprising a gamma-emitting radionuclide conjugated to a second binding agent that selectively binds to a second cellular marker.

62. A method of performing intraoperative image-guided surgery on a subject, the method comprising:
administering a detectably effective amount of at least one imaging agent to the subject, wherein the at least one imaging agent comprises a fluorophore and a gamma-emitting radionuclide conjugated to a binding agent that selectively binds to at least one of a cellular marker on a lesion, a lymphatic marker, a vessel marker, and a nerve marker;
illuminating tissue with light at a fluorescence excitation wavelength of the fluorophore; and performing image-guided surgery using a dual modality imager comprising:
a first chip comprising an array of gamma photon-sensing pixels for imaging the gamma-emitting radionuclide, wherein each gamma photon-sensing pixel provides in-pixel signal amplification for incoming gamma photons that hit the gamma photon-sensing pixel and in-pixel data processing to determine relative energies and angles of incidence of the incoming gamma photons, and wherein each gamma photon-sensing pixel only outputs data from the in-pixel data processing in response to an incoming gamma photon hitting the gamma photon-sensing pixel; and
a second chip comprising an array of fluorescence-sensing pixels for imaging the fluorophore, wherein the second chip is positioned relative to the first chip such that the incoming gamma photons pass through the second chip to reach the array of gamma photon-sensing pixels, wherein the dual modality imager produces fluorescence and radionuclide images of a surgical site comprising the at least one of the lesion, the lymphatic marker, the vessel marker, and the nerve marker.

63. The method of claim 90, wherein the binding agent selectively binds to the cellular marker on the lesion, the surgery is resection of the lesion, and the method further comprises illuminating a surgical specimen removed from the subject with light at a fluorescence excitation wavelength of the fluorophore;
moving the dual modality imager over surgical margins surrounding the lesion in the surgical specimen, wherein fluorescence and radionuclide images produced by the dual modality imager are used to detect cells of the lesion, if present, in the surgical margins to determine if the surgical specimen comprises a positive margin; and
further checking the tissue surrounding the resection site in the subject for the cells of the lesion if the surgical specimen is determined to comprise a positive margin.

64. The method of claim 62, wherein the lesion is a tumor, diseased tissue, a congenital malformation, or damaged or abnormal tissue.

65. The method of any claim 62, wherein the binding agent is an antibody, an antibody mimetic, an aptamer, or a ligand.

66-67. (canceled)

68. The method of claim 62, further comprising administering a plurality of imaging agents to the subject, wherein each imaging agent comprises a different binding agent that selectively binds to a different marker, and wherein each imaging agent comprises a different fluorophore.

69. The method of claim 62, further comprising obtaining a pre-operative image of the subject after the subject is administered an imaging agent comprising a gamma-emitting radionuclide conjugated to a binding agent that selectively binds to the cellular marker on the lesion, wherein the pre-operative image shows locations of the gamma-emitting radionuclide in tissue of the subject.

70. The method of claim 62, further comprising attaching a gamma photon counter to the skin of the subject, wherein the gamma photon counter is used in combination with the dual modality imager to determine the location of the radionuclide source of the incoming gamma photons that hit a gamma photon-sensing pixel.

71. The method of claim 62,
wherein the binding agent selectively binds to the lymphatic marker; and
wherein the dual modality imager produces fluorescence and radionuclide images of the lymphatic system.

72-81. (canceled)

82. The method of claim 62, further comprising:
moving the dual modality imager over a surgical site to produce additional fluorescence and radionuclide images of the surgical site after performing the surgery; assessing results of the surgery using the fluorescence and radionuclide images of the surgical site; and
performing further surgery on the tissue based on assessing the results of the surgery.

83-85. (canceled)

86. The method of claim 62, wherein the binding agent selectively binds to the vessel marker that is an artery marker or a vein marker;
wherein the dual modality imager produces fluorescence and radionuclide images of an artery or vein.

87-89. (canceled)

90. The method of claim 62, wherein the binding agent selectively binds to the cellular marker on the lesion, and the dual modality imager provides information regarding tissue depths and sizes of the lesion.
